(19) United States
(12) Patent Application Publication
(10) Pub. No.: US 2017/0035943 A1
(43) Pub. Date: Feb. 9, 2017

(54) IMPLANTABLE GRAPHENE MEMBRANES WITH LOW CYTOTOXICITY

(71) Applicant: Lockheed Martin Corporation, Bethesda, MD (US)

(72) Inventors: Sarah M. SIMON, Baltimore, MD (US); Jacob L. Swett, Redwood City, CA (US); Peter V. Bedworth, Los Gatos, CA (US); Scott E. Heise, San Jose, CA (US)

(73) Assignee: Lockheed Martin Corporation, Bethesda, MD (US)

(21) Appl. No.: 15/099,193

(22) Filed: Apr. 14, 2016

Related U.S. Application Data
(60) Provisional application No. 62/202,056, filed on Aug. 6, 2015.

(51) Int. Cl. A61L 31/02; A61L 31/00; A61L 31/10; A61L 31/16; A61L 31/08; A61L 31/06; A61L 31/04

(52) U.S. Cl. CPC A61L 31/024; A61L 31/06; A61L 31/005; A61L 31/048; A61L 31/16; A61L 31/084; A61L 31/10; A61L 2300/64; A61L 2420/08

(57) ABSTRACT

Two-dimensional materials can be formed into enclosures for various substances and a substrate layer can be provided on an outside and/or on an inside of the enclosure, wherein the enclosure is not cytotoxic. The enclosures can be exposed to an environment, such as a biological environment (in vivo or in vitro), where the fibrous layer can promote vascular ingrowth. One or more substances within the enclosure can be released into the environment, one or more selected substances from the environment can enter the enclosure, one or more selected substances from the environment can be prevented from entering the enclosure, one or more selected substances can be retained within the enclosure, or combinations thereof. The enclosure can, for example, allow a sense-response paradigm to be realized. The enclosure can, for example, provide immunoisolation for materials, such as living cells, retained therein.

### CROSS-REFERENCE TO RELATED APPLICATIONS

This application claims the benefit of U.S. provisional application 62/202,056, filed Aug. 6, 2015, which is incorporated by reference herein in its entirety.

### BACKGROUND

Drug and cell delivery in both immune competent and immune incompetent organisms is a problem in medical research and practice today. Recent studies use polymeric devices and hydrogels as a delivery vehicle. Some examples include polytetrafluoroethylene (e.g., expanded PTFE) with a backing of unwoven polyester mesh, silicone, hydrogels, alginate, cellulose sulfate, collagen, gelatin, agarose, chitosan and the like. Current delivery vehicles and devices are challenged by biofouling, biocompatibility issues, and a lengthy diffusion time of substances out of the vehicle. The thickness of current state devices can limit efficacy, due in part to limited diffusion of nutrients into the devices and/or impeded transfer of substances into and out of the device. Low permeability, at least in part, due to thickness and mechanical stability in view of physical stress and osmotic stress can also be problematic. Moreover, replicating the cellular walls, selective channels, and the semi-permeance that biological membranes provide has long proven to be a challenge for synthetic membranes or semi-permeable walls, especially when integrating those membranes in vitro or in vivo. In addition, current membranes insufficiently achieve immunoisolation, especially in the context of xenographic, allogenic, and autogenic transplants.

In view of the foregoing, improved techniques for transportation, delivery, separation, and forming selective barriers of substances under a variety of conditions, including in a biological environment, would be of considerable benefit in the art.

### SUMMARY

Some embodiments comprise enclosures comprising a compartment and a wall separating the compartment from an environment external to the compartment, wherein the wall comprises: a perforated graphene-based material layer and a substrate layer, and wherein the enclosure is not cytotoxic when implanted into a subject. In some embodiments, the substrate layer is affixed directly to perforated graphene-based material. In some embodiments, the substrate layer is affixed indirectly to the perforated graphene-based material. In some embodiments, the substrate layer comprises track-etched polyimide. In some embodiments, the substrate layer comprises a fibrous layer comprising a plurality of polymer filaments. In some embodiments, the compartment is in fluid communication with the external environment. In some embodiments, at least a portion of the wall is from about 5 nm to about 1 μm thick. Some embodiments further comprise at least one substance encapsulated within the compartment. Some embodiments comprise two or more substances are encapsulated within the compartment. In some embodiments, the substance comprises one or more cells, and the perforated graphene has pores with a size sufficient to retain the cell within the compartment and to exclude immune cells and immune complexes in the environment external to the compartment from entering the compartment. In some embodiments, the cells are yeast or bacterial cells. In some embodiments, the cells are mammalian cells.

In some embodiments, the compartment does not contain sub-compartments. In some embodiments, the compartment comprises two or more sub-compartments. In some embodiments, one or more sub-compartments are separated from an environment external to the sub-compartment. In some embodiments, the one or more sub-compartments are separated from the environment external to the sub-compartment by a wall comprising a perforated graphene-based material.

Some embodiments comprise perforated graphene comprising pores with a size sufficient to allow a pharmaceutical to pass between the compartment and the external environment. In some embodiments, the perforated graphene-based material has pores with a size of from about 1 nm to about 10 nm. Pore sizes in a wall of one sub-compartment can be the same as or different from pore sizes in a wall of a different sub-compartment. In some embodiments, the graphene-based material is graphene. Pore sizes in the perforated graphene can be tailored to selectively exclude various substances based, e.g., on size. See, for instance FIG. 18, showing differences in sizes of exemplary substances that can be excluded from traversing the perforated graphene based on the pore size. FIGS. 30-32 show images demonstrating the ability to tune pore sizes, for instance via dilation, e.g., with He²⁺ or Xe⁺ ion beams. FIG. 31 further demonstrates the ability to consistently introduced pores over relatively large areas.

Some embodiments comprise a substrate layer positioned on a compartment-facing side of the wall, on an exterior surface of the wall, or on both the compartment-facing side and exterior surface of the wall. In some embodiments, the substrate layer is disposed on the compartment-facing side of the graphene-based material, the external side of the graphene-based material, or both. In some embodiments, the substrate layer has a thickness of about 1 mm or less.

In some embodiments, the substrate layer comprises a plurality of pores. In some embodiments, the substrate layer has a porosity gradient throughout its thickness. In some embodiments, the substrate layer is hydrophobic or hydrophilic. In some embodiments, the fibrous substrate comprises a material selected from the group consisting of polysulfones, polyurethane, polymethylmethacrylate (PMMA), polyglycolid acid (PGA), polylactic acid (PLA), polyethylene glycol (PEG), polylactic-co-glycolic acid (PLGA), polyamides, polyimides, polypropylene, polyethersulfones (PES), polyvinylidine fluoride (PVDF), cellulose acetate, polyethylene, polypropylene, polycarbonate, polytetrafluoroethylene (PTFE), polyvinylchloride (PVC), polyether ether ketone (PEEK), block co-polymers of any of these, and combinations and/or mixtures thereof.

In some embodiments, the substrate layer comprises an additive selected from the group consisting of pharmaceuticals, cells, growth factors, clotting factors, blood thinners, immunosuppressants, antimicrobial agents, hormones, antibodies, antigens or an antibody-binding fragment thereof, minerals, nutrients, and combinations thereof.

In some embodiments, the enclosures comprise an intermediate layer positioned between the perforated graphene-based material layer and the substrate layer.

Some embodiments comprise methods of releasing a substance comprising exposing an enclosure comprising a wall with a perforated graphene-based material layer and a substrate layer to an environment, to thereby release into the environment at least one substance from a compartment in the enclosure, wherein the enclosure is not cytotoxic to the environment. In some embodiments, the environment is a biological environment. In some embodiments, the substance is a pharmaceutical. In some embodiments, the compartment contains cells which are not released from the enclosure. In some embodiments, the cells produce the substance released from the enclosure.

Some embodiments comprise exposing an enclosure comprising a perforated graphene-based material to an environment, to thereby release at least one first substance into the environment and to allow passage of a second substance from the environment into the enclosure, wherein the perforated graphene based material is not cytotoxic to the environment. In some embodiments, the first substance is cells and the second substance is a nutrient or oxygen. In some embodiments, a substance that enters the enclosure can return to the environment.

Some embodiments comprise a composite structure comprising: perforated graphene-based material and a substrate layer comprising a plurality of polymer filaments affixed directly or indirectly to at least one surface of the perforated graphene-based material, wherein the composite structure is substantially planar, and wherein the composite structure is not cytotoxic when implanted into a subject. In some embodiments, the composite structure further comprises a second substrate layer affixed directly or indirectly to a surface of the perforated graphene-based material opposite the first substrate layer. In some embodiments, the first and/or second substrate layer comprises an additive selected from the group consisting of pharmaceuticals, cells, growth factors, clotting factors, blood thinners, immunosuppressants, antimicrobial agents, hormones, antibodies, antigens or an antibody-binding fragment thereof, minerals, nutrients and combinations thereof. In some embodiments, the composite structure is flexible.

Some embodiments comprise enclosures comprising a compartment and a wall separating the compartment from an environment external to the compartment, wherein the wall comprises: a perforated graphene-based material layer and a means for enhancing integration of the enclosure into tissue and/or vascularization to the enclosure, wherein the enclosure is not cytotoxic when implanted into a subject.

Some embodiments comprise methods of preparing an enclosure comprising a compartment and a wall separating the compartment from an environment external to the compartment, wherein the wall comprises: a perforated graphene-based material layer and a substrate layer comprising a plurality of polymer filaments.

Some embodiments comprise methods of improving biocompatibility of an enclosure comprising a perforated graphene-based material, wherein the method comprising affixing a substrate layer comprising a plurality of polymer filaments to the outside of the enclosure.

Some embodiments comprise methods of preparing an enclosure that is not cytotoxic when implanted into a subject, wherein the enclosure comprises a compartment and a wall separating the compartment from an environment external to the compartment, wherein the wall comprises a perforated graphene-based material layer. Some embodiments comprise methods of preparing an enclosure that is not cytotoxic when implanted into a subject, wherein the enclosure comprises a compartment and a wall separating the compartment from an environment external to the compartment, wherein the wall comprises a perforated graphene-based material layer and a substrate layer. In some embodiments, the substrate layer comprises track-etched polyimide. In some embodiments, the substrate layer comprises a fibrous layer comprising a plurality of polymer filaments.

Some embodiments comprise methods of improving biocompatibility of an enclosure comprising a perforated graphene-based material, wherein the method comprising affixing a substrate layer to the outside of the enclosure, wherein the enclosure is not cytotoxic when implanted into a subject.

Some embodiments comprise methods of treating diabetes comprising implanting into a subject an enclosure comprising a perforated graphene-based material layer and a substrate layer comprising a plurality of polymer filaments, wherein the enclosure is not cytotoxic to the subject.

Some embodiments comprise methods of reducing cytotoxicity of a device comprising encapsulating the device with a composite structure comprising (a) a perforated graphene-based material layer and (b) a fibrous layer comprising a plurality of polymer filaments affixed directly or indirectly to at least one surface of the perforated graphene-based material, wherein the device has a reduced cytotoxicity as compared to a comparable device not encapsulated by the composite structure.

Some embodiments comprise coated therapeutic devices comprising: (i) a therapeutic device and (ii) a coating on the therapeutic device, wherein the coating comprises a composite structure comprising (a) a perforated graphene-based material layer and (b) a fibrous layer comprising a plurality of polymer filaments affixed directly or indirectly to at least one surface of the perforated graphene-based material. In some embodiments, the coated therapeutic device of has a lower toxicity than a comparable therapeutic device that is not coated with the composite structure.

Some embodiments comprise coated therapeutic devices comprising: (a) a therapeutic device and (b) a coating on the therapeutic device, wherein the coating comprises a perforated graphene-based material layer. In some embodiments, the coated therapeutic device has a lower toxicity than a comparable therapeutic device that is not coated with the composite structure.

Some embodiments include biologically relevant devices that provide a suitable substrate to enable deposition, lamination or transfer of a semi-permeable material to promote an integrated vasculature for sustained growth and survival of cells inside a semipermeable enclosure. In some embodiments, a semi-permeable 2D material (e.g. perforated graphene) is affixed to one or more fibrous layers. This composite structure provides for tissue integration or ingrowth, enhances transport or passage, and/or minimizes the impact on the passage of semi-permeance to metabolites, immune factors, cells and the like. The proximity of the vasculature and the encapsulated substance allows for the facile passage of permeable metabolites between the blood and the contents of the enclosure.

Enclosures formed from perforated graphene or other perforated two-dimensional materials are disclosed in U.S. application Ser. No. 14/656,190, which is hereby incorporated by reference in its entirety. The enclosures can house various substances therein allowing movement of selected substances to and from the interior of the enclosure, retaining other selected substances therein and preventing entry of yet other selected substances into the enclosure. The enclosure can be employed to release one or more selected substances into an environment external to the enclosure, to allow entry into the enclosure of one or more selected substances from an environment external to the enclosure, to inhibit and preferably prevent entry of one or more selected substances from the external environment into the enclosure, to retain (inhibit or preferably prevent exit of) one or more selected substances within the enclosure or a combination of these applications. The hole or aperture size or range of sizes in a perforated material of the enclosure can be selected based on the specific application of the enclosure. The term enclosure refers to a space for receiving one or more substances, where the enclosure is formed, at least in part, by a perforated two-dimensional material, such as a graphene-based material, where, in some embodiments, one or more substances in the enclosure can exit the enclosure by passage through the perforated two-dimensional material. Similarly, in some embodiments, one or more substances from the external environment can enter the enclosure by passage through the perforated two-dimensional material. In some embodiments the external environment is a biological environment, which may be an in vivo biological environment or an in vitro biological environment. In some embodiments, the size and/or properties of perforations for each sub-compartment can be the same as and or different from the size and/or properties of perforations in a different sub-compartment.

In some embodiments, an enclosure comprises one or more than one sub-compartments each sub-compartment comprising perforated two-dimensional material such that at least a portion of the walls or sides forming the sub-compartment are perforated two-dimensional material. Fluid communication is achieved by selective passage of one or more substances in and/or out of the enclosure or sub-compartment. Thus, compartments and sub-compartments separated from an external environment can be in fluid communication with the external environment. The fluid can be liquid or gas and includes fluids having entrained gases. Substances can be dissolved or suspended or otherwise carried in a fluid. The fluid can be aqueous. A sub-compartment can be in direct fluid communication with adjacent sub-compartments and/or the external environment (where adjacent sub-compartments share at least one wall or side). In some embodiments one or more sub-compartments can be in direct fluid communication with adjacent sub-compartments, but not in direct fluid communication with the external environment. At least one sub-compartment in an enclosure is in direct fluid communication with an external environment. An enclosure can have various configurations of sub-compartments. A sub-compartment can have any shape. A sub-compartment may, for example, be spherical, cylindrical or rectilinear. In some embodiments, sub-compartments can be nested. In some embodiments, the enclosure can have a central sub-compartment which shares a wall or side with a plurality of surrounding sub-compartments. In some embodiments, sub-compartments can be linearly aligned within the enclosure. In some embodiments, an enclosure contains two sub-compartments. In some embodiments, an enclosure contains three, four, five or six sub-compartments. In some embodiments, a sub-compartment can be fully contained within another sub-compartment, wherein the inner sub-compartment is in direct fluid communication with the outer sub-compartment and the outer-sub-compartment is in direct fluid communication with the external environment. In this embodiment, the inner sub-compartment is in indirect rather than direct fluid communication with the external environment. In some embodiments where an enclosure contains a plurality of sub-compartments or is divided into a plurality of sub-compartments, at least one sub-compartment is in direct fluid communication with the external environment and remaining sub-compartments are in direct fluid communication with adjacent sub-compartments, but may not all be in direct fluid communication with the external environment. In some embodiments where an enclosure contains a plurality of sub-compartments, all sub-compartments can be in direct fluid communication with the external environment.

An enclosure encapsulates at least one substance. In some embodiments, an enclosure can contain two or more different substances. Different substances can be in the same or in different sub-compartments. In some embodiments, not all of the different substances in the enclosure are released to an environment external to the enclosure. In some embodiments, all of the different substances in the enclosure are released to an external environment. In some embodiments, the rate of release of different substances from the enclosure into an external environment is the same. In some embodiments, the rates of release of different substances from the enclosure into an external environment are different. In some embodiments, the relative amounts of different substances released from the enclosure can be the same or different. The rate of release of substances from the enclosure can be controlled by choice of hole size, hole geometry, hole functionalization or combinations of these.

Methods for transporting and delivering substances in a biological environment are also described herein. In some embodiments, the methods can include introducing an enclosure formed from graphene or other two-dimensional material into a biological environment, and releasing at least a portion of a substance in the enclosure to the biological environment. In some embodiments, methods can include introducing an enclosure formed from graphene or other two-dimensional material into a biological environment, and allowing migration of a substance from the biological environment into the enclosure.

In some embodiments, an enclosure comprises perforated two-dimensional material forming at least one wall of the enclosure, wherein the enclosure is separated from an environment external to the enclosure, and a substrate layer disposed on the two-dimensional material. In some embodiments, the enclosure comprises two or more sub-compartments. In some embodiments, an enclosure or sub-compartment is “substantially sealed” from an environment external to the enclosure or sub-compartment when passage of substances into or out of the enclosure or sub-compartment occurs almost exclusively (i.e., at least 95%) through defects, holes or apertures in the plane of a perforated two-dimensional material. In particular, edges of the perforated two-dimensional material are “substantially sealed” when passage of substances into or out of the enclosure or sub-compartment occurs almost exclusively (i.e., at least 95%) through defects, holes or apertures in the plane of a perforated two-dimensional material. Degree of sealing can be calculated, for instance, based on an integrity test using a control enclosure produced with or without perforations.

At least one substance can be encapsulated within the enclosure, such that the at least one substance is released to an environment external to the enclosure by passage through holes in the perforated two-dimensional material. In some embodiments, at least once substance within the enclosure (e.g., cells) produces a second substance (e.g., insulin) that can be released to an environment external to the enclosure by passage through holes in the perforated two-dimensional material. In some embodiments, two or more different substances are encapsulated within the enclosure. In some embodiments, the substance within the enclosure that is released to an environment external to the enclosure through holes in the two-dimensional material can be a pharmaceutical. In some embodiments, the substance within the enclosure is cells and the size of the holes in the two-dimensional material is selected to retain the cells within the enclosure and to possibly exclude immune cells and immune complexes from entering the enclosure from the environment external to the enclosure. In some embodiments, the substance within the enclosure is cells and the size of the holes in the two-dimensional material is selected to retain the cells within the enclosure while immune cells and immune complexes from the environment external to the enclosure can enter the enclosure. For example, the cells can be stem cells, yeast cells, bacterial cells, or mammalian cells.

In some embodiments, the size of the holes in the two-dimensional material ranges from 1-50 nm, 1-40 nm, 1-30 nm, 1-25 nm, 1-17 nm, 1-15 nm, 1-12 nm, 1-10 nm, 3-50 nm, 3-30 nm, 3-20 nm, 3-10 nm, or 3-5 nm. In some embodiments, the size of the holes is about 1 nm, about 3 nm about 5 nm, about 10 nm, about 15 nm, about 20 nm, about 30 nm, or about 50 nm. In some embodiments, the two-dimensional material is graphene or a graphene-based material.

In some embodiments, the polymer filaments are disposed on an inside of the enclosure, an outside of the enclosure or both an inside and an outside of the enclosure. In some embodiments, the substrate layer has a thickness of 1 mm or less, about 100 μm or less, about 10 μm or less, about 1 μm or less, or about 10 nm or less. In some embodiments, the substrate layer has a thickness of about 1 nm to about 100 nm, about 2 nm to about 5 nm, about 5 nm to about 10 nm, or about 20 nm to about 50 nm. In some embodiments, polymer filaments are applied to the two-dimensional material by a wet electrospinning process or a dry electrospinning process, where the fibers dry before hitting the two-dimensional material, such that a plurality of pores is introduced in the fibrous layer as the fibrous layer is deposited. The fibrous layer may or may not have a porosity gradient throughout its thickness. In some embodiments, the substrate layer comprises a material selected from the group consisting of polysulfones, polyurethane, polymethylmethacrylate (PMMA), polyglycolid acid (PGA), polylactic acid (PLA), polyethylene glycol (PEG), polylactic-co-glycolic acid (PLGA), polyamides (such as nylon-6,6, supramid and nylamid), polyimides, polypropylene, polyethersulfones (PES), polyvinylidine fluoride (PVDF), cellulose acetate, polyethylene, polypropylene, polycarbonate, polytetrafluoroethylene (PTFE) (such as Teflon), polyvinylchloride (PVC), polyether ether ketone (PEEK) and mixtures and block co-polymers thereof. In some embodiments, the materials used to make the substrate layer are hydrophobic and/or hydrophilic. In some embodiments, materials used to make the substrate layer are highly pure, contain no solvents, and/or are of a medical grade.

In some embodiments, the substrate layer comprises an additive selected from the group consisting of pharmaceuticals, cells, growth factors, clotting factors, blood thinners, immunosuppressants, antimicrobial agents, hormones, antibodies, antigens or an antibody-binding fragment thereof, minerals, nutrients and combinations thereof. In some embodiments, the additive can have a concentration gradient within the substrate layer (e.g., along a Z-direction, where the two-dimensional material defines the X-Y plane). For example, a concentration of the additive can be greater or lesser proximal to the two-dimensional material than distal to the two-dimensional material. Such a gradient can be achieved, for example, by altering a concentration of one or more additive compounds during an electrospinning process. In some embodiments, the gradient can be achieved by soaking the substrate layer in a bath, e.g., where the fluid only rises to a certain level of the substrate (i.e., it does not cover the entire substrate).

In some embodiments, the enclosure further comprises an intermediate layer disposed between the two-dimensional material layer and the substrate layer.

Some embodiments comprise a method comprising: introducing an enclosure comprising perforated two-dimensional material and a fibrous layer comprising a plurality of polymer filaments disposed on the two-dimensional material to an environment, the enclosure containing at least one substance; and releasing at least a portion of the at least one substance through the holes of the two-dimensional material to the environment external to the enclosure. In some embodiments, the environment is a biological environment. In some embodiments, the at least one substance, a portion of which is released, is a drug. In some embodiments, the enclosure contains cells which are not released from the enclosure and the at least one substance, a portion of which is released, is a substance generated by the cells in the enclosure. Any enclosure herein can be employed in this method.

In some embodiments, the method comprises: introducing an enclosure comprising perforated two-dimensional material to a environment, the enclosure containing at least one first substance; and allowing migration of a second substance from the environment into the enclosure. Any enclosure herein can be employed in this method.

In some embodiments, the first substance is cells and the second substance is a nutrient or oxygen.

In some embodiments, upon reaction or complexation of the first substance within the enclosure with the second substance that enters the enclosure, the second substance is substantially trapped inside the enclosure and inhibited from returning to the environment external to the enclosure. For example, a chemical complex of the first and second substance can be larger than the average pore size of the two-dimensional material such that less than 10%, less than 5%, less than 2% or less than 1% of the second substance returns to the environment external to the enclosure.

In some embodiments, the method comprises: introducing an enclosure comprising perforated two-dimensional material to an environment, the enclosure containing at least one substance; and releasing at least a portion of the at least one substance through the holes of the two-dimensional material to the environment external to the enclosure. Any enclosure herein can be employed in this method.

In some embodiments, the method comprises: introducing an enclosure comprising perforated two-dimensional material to an environment, the enclosure containing at least one first substance; and allowing migration of a second substance from the environment into the enclosure. In some embodiments, the first substance is cells, the second substance is nutrients and another second substance is oxygen. Any enclosure herein can be employed in this method.

In some embodiments, a composite structure comprises perforated two-dimensional material; and a first fibrous layer comprising a plurality of polymer filaments affixed to a surface of the two-dimensional material; wherein the composite structure is substantially planar. In some embodiments, the perforated two-dimensional material has a second fibrous layer affixed to a surface of the two-dimensional material opposite the first fibrous layer. In some embodiments, the first and/or second fibrous layer comprises an additive selected from the group consisting of pharmaceuticals, cells, growth factors, clotting factors, blood thinners, immunosuppressants, antimicrobial agents, hormones, antibodies, minerals, nutrients and combinations thereof. In some embodiments, the substantially planar composite structure is flexible.

### BRIEF DESCRIPTION OF THE DRAWINGS

FIG. 1 is a schematic of some embodiments of graphene.

FIGS. 2A-2E show illustrative schematics of some embodiments with various configurations of enclosure configurations comprising a two-dimensional material.

FIGS. 3A and 3B are schematic illustrations of some embodiments of an enclosure implemented for immunoisolation of living cells.

FIGS. 4A-4C illustrate some embodiments for preparing an enclosure.

FIG. 5 shows an illustrative SEM micrograph of some embodiments with a plurality of electrospun PVDF fibers deposited on graphene.

FIG. 6 shows an illustrative schematic of some embodiments of a composite structure comprising a two-dimensional material and a two fibrous layers. The fibrous layers allow for capillary ingrowth that brings the blood supply close to the two-dimensional material to facilitate exchange of molecules with the cells, proteins, tissue or the like on the opposite side of the two-dimensional material. SEM micrographs show embodiments with fibrous layers that have different pore sizes.

FIG. 7 shows schematic illustrations of some embodiments of composite structures comprising two-dimensional materials (e.g., graphene), an optional intermediate layer (e.g., track etched polymer membrane), and a fibrous layer having a tighter fiber spacing nearer the two-dimensional material and an increasing effective pore size further from the two-dimensional material. FIG. 7A also shows SEM micrographs from various locations in the composite structure. Also included are variants with direct substrate deposition (7B), hybrid thin membrane+deposition (7C), hybrid substrate deposition+thin film polymer sandwiching a graphene layer (7D) and hybrid substrate position+thin polymer film on the same side of a graphene layer.

FIG. 8 shows an illustrative schematic of some embodiments of corrugation of graphene or graphene-based material after chemical vapor deposition on a planar growth substrate (1) by: pressing the graphene or graphene-based material and growth substrate onto a corrugated template (2); followed by application of an electrospun fibrous layer (3); removal of the graphene or graphene-based material, growth substrate and fibrous layer from the template (4); and etching of the growth substrate (5), to produce graphene or graphene-based material on electrospun material with a high surface area (6).

FIG. 9 shows an illustrative schematic of some embodiments of a corrugated cylindrical workpiece for receiving graphene or graphene-based material on a growth substrate, as shown in FIG. 8. Electrospray deposition of a fibrous layer on a non-rotated or planar surface produces a randomly distributed fibrous layer, whereas rotation of a cylindrical workpiece during the electrospray process produces an aligned fibrous layer. In the figure, entire outside of the cylinder is corrugated.

FIG. 10 shows an illustrative schematic of some embodiments of a process for manufacturing a two-dimensional material on a fibrous layer with mesh reinforcement.

FIG. 11 shows a SEM micrograph of some embodiments of two layers of graphene or graphene-based material on a fibrous layer.

FIG. 12 shows SEM micrographs of some embodiments of single-layer or two-layer graphene on a substrate at various magnifications and using two different electrospinning recipes, as set forth in the figure. In both recipes, 7% nylon 6,6 was electrospun and graphene was transferred to the electrospun layer. Arrows in the figure demonstrate defects and/or areas where the graphene drapes.

FIG. 13 shows a high-magnification SEM micrograph of some embodiments of single-layer graphene on a substrate prepared according to recipe 1 in FIG. 12.

FIG. 14A shows micrographs of some embodiments of substrate layers (e.g., termed “tortuous path membrane” and “track etched membrane” in the figure); a two-dimensional membrane; and a composite structure with both a substrate layer and a two-dimensional membrane layer. FIG. 14B illustrates exemplary embodiments of materials subjected to cytotoxicity testing and implantation testing. FIG. 14C shows photographs of some embodiments of exemplary test materials used in cytotoxicity studies.

FIG. 15 illustrates some embodiments for using graphene in methods of immunoisolation.

FIG. 16 illustrates some embodiments for preparing and using graphene in methods of immunoisolation.

FIG. 17 illustrates some embodiments of devices prepared with a composite structure (e.g., a substrate layer+a perforated graphene layer).

FIG. 18 illustrates examples of substances that can be selectively excluded by two-dimensional graphene in some embodiments, based on the size of the pores in the two-dimensional graphene (graphics not to scale). All sizes set forth in the figure are approximate.

FIG. 19 shows some embodiments of graphene disposed on various substrates, including: track-etched polyimide, track-etched polycarbonate, microporous SiN, electrospun membrane, PVDF microfiltration membrane, nanoporous SiN, carbon nanomaterial membrane, and SiN microseive. The right-most sub-figures show increased magnification views of graphene on an SiN microporous substrate.

FIG. 20 shows micrographs of some embodiments of custom track-etched polyimide (TEPI).

FIG. 21 shows a micrograph of some embodiments of graphene disposed on track-etched polyimide.

FIG. 22 a micrograph of some embodiments of perforated graphene disposed on track-etched polyimide. The right side of the figure is an increased magnification view that corresponds with the white box on the left side of the figure.

FIG. 23 shows a micrograph of some embodiments of graphene disposed on electrospun nylon 6,6.

FIG. 24 shows a magnified view of the micrograph shown in FIG. 23.

FIG. 25 shows diffusive transport of small (Allura Red AC) and large (silver nanoparticles) analytes across a substrate layer and a perforated graphene layer of some embodiments as compared to a control. In the graph, the lighter-shaded bars correspond to permeability of Allura Red AC, and the darked-shaded bars correspond to the ration of Allura Red AC permeability:silver nanoparticle permeability. The figure also includes a picture of a device that can be used to test diffusive transport.

FIG. 26 shows data related to normalized diffusive transport of fluorescein conjugated to immunoglobulin-G (IgG) across a SiN substrate layer, a control membrane (Biopore), a perforated graphene layer, and an unperforated graphene layer. In the bar graph, the order of bars from left to right correspond with: (i) Bare Chip (left-most bar), (ii) perforated graphene, (iii) unperforated graphene (bar with lowest flux), and (iv) Biopore (right-most bar). In the line graph, the lines correspond, from highest IgG concentration to lower IgG concentration, with: (i) Uncoated chip, (ii) perforated graphene, (iii) Biopore membrane, and (iv) unperforated graphene (represented by dots).

FIG. 27 shows data related to permeability of fluorescein across perforated graphene (line with highest analyte concentration in the graph) and a control membrane (Biopore) (line with second highest analyte concentration in the graph), and permeability of IgG across perforated graphene (line with 3^{rd} highest analyte concentration in the graph) and a control membrane (line with lowest analyte concentration in the graph).

FIG. 28 shows photographs of some embodiments where 100 nm diameter Red (580/605) FluoroSpheres are restricted from traversing perforated graphene, but fluorescein is able to traverse the perforated graphene.

FIG. 29A shows data related to permeability of fluorescein across various substrate layers, substrate layers coated with perforated graphene, and unperforated graphene. In the figure, the left-most section relates to permeability experiments conducted on a control membrane (Biopore); the middle section relates to permeability experiments conducted with uncoated substrate TEPI-400/7 (the left two bars in the middle section) and TEPI-400/7 coated with 2 layers of unperforated graphene (the right-most bar in the middle section); the right-most section relates to permeability experiments conducted with uncoated substrate TEPI-460/25 (the left five bars), TEPI-460/25 coated with 2 layers of perforated graphene (the two bars with the lowest permeability), and TEPI-460/25 coated with perforated graphene (the right six bars). FIG. 29B shows data related to permeability of fluorescein across an uncoated substrate, across a perforated graphene-coated substrate, and across an unperforated graphene-coated substrate. FIG. 29C shows data related to permeability of FluoroSphere across an uncoated substrate and a graphene-coated substrate.

FIG. 30 shows TEM images of some embodiments of perforated graphene with various pore sizes. The two left-most images show perforated graphene with relatively small pores; the two right-most images show perforated graphene with relatively large pores. In the figure, perforated graphene in the middle micrograph on the bottom and the right-most micrograph on the bottom were dilated using Xe ion irradiation.

FIG. 31 shows SEM images of some embodiments showing consistent perforation of graphene over relatively large areas.

FIG. 32 shows SEM images of some embodiments showing the ability to tune pore sizes in perforated graphene via dilation.

### DETAILED DESCRIPTION

Some embodiments relate to the selective passage of substances through an enclosure that encourages nearby vascularization (i.e., angiogenesis) and/or tissue ingrowth in a biological environment. Some embodiments include methods and devices for selectively separating or isolating substances in a biological environment, e.g., using a composite structure that comprises a two-dimensional material. Some embodiments include an enclosure comprising a compartment and a wall separating the compartment from an environment external to the compartment. In some embodiments, the wall comprises a two-dimensional material layer and a substrate layer. Two-dimensional materials, such as graphene-based materials, are discussed below.

Enclosures can be in any shape. Thus, the cross-section of an enclosure can be, for example, circular, ovular, rectangular, square, or irregular-shaped. The size of the enclosure also is not limited, and can be small enough to circulate in the bloodstream (e.g., on the order of nanometers or larger) or large enough for implantation (e.g. on the order of inches or smaller). In some embodiments, the enclosure is from 100 nm to 6 inches long in its longest dimension, such as from about 100 nm to about 500 nm, about 500 nm to about 1 μm, about 1 μm to about 500 μm, about 500 μm to about 1 mm, about 1 mm to about 500 mm, about 500 mm to about 1 cm, about 1 cm to about 10 cm, or about 1 cm to about 6 inches long. In some embodiments, the enclosure is longer than 6 inches in its longest dimension, such as about 10 inches or about 15 inches long.

The thickness of the wall depends, in part, on the two-dimensional material layer and/or substrate layers used in the wall. Thus, in some embodiments a wall, or a portion thereof, comprising both a two-dimensional material layer and a substrate layer is at least 5 nm thick, such as from about 5 nm to about 1 μm thick, from about 5 nm to about 250 nm thick, from about 5 to about 50 nm thick, from about 5 to about 20 nm thick, or from about 20 to about 50 nm thick. In some embodiments, the thickness of the wall is about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm thick, about 25 nm thick, about 30 nm thick, about 35 nm thick, about 40 nm thick, about 45 nm thick, about 50 nm thick, about 100 nm thick, about 150 nm thick, about 200 nm thick, about 250 nm thick, about 300 nm thick, about 400 nm thick, about 500 nm thick, about 600 nm thick, about 700 nm thick, about 800, nm thick, about 900 nm thick, or about 1 μm thick. In some embodiments, the thickness of the wall is up to about 1 μm thick or up to about 1 mm thick. In some embodiments, the thickness of the wall is tailored to allow bidirectional passage of oxygen and nutrients into and out of the enclosure. In some embodiments, the thickness of the wall is tailored to allow entry of oxygen and nutrients into the enclosure at sufficient concentrations to maintain viability of cells within the enclosure.

In some embodiments, the substrate layer has a thickness of about 1 mm or less, about 1 μm or less, or about 100 nm or less. In some embodiments, a thickness of the substrate layer can range from about 100 nm to about 100 μm, or about 1 μm to about 50 μm, or about 10 μm to about 20 μm, or about 15 μm to about 25 μm. In some embodiments, the substrate layer has a thickness about 10 μm or greater, or about 15 μm or greater. In some embodiments, the substrate layer has a thickness of less than 1 μm. In some embodiments, the substrate layer has a thickness of about 10 nm to about 100 nm, or about 20 nm to about 50 nm.

In some embodiments, the enclosure can be supported by one or more support structures. In some embodiments, the support structure can itself have a porous structure wherein the pores are larger than those of the two-dimensional material. In some embodiments, the support structure is formed as a frame at a perimeter of a two-dimensional material. In some embodiments, the support structure is positioned in part interior to a perimeter of a two-dimensional material. In some embodiments, the substrate layer can convey a desired degree of structural support (e.g., to prevent tearing and/or buckling) to the two-dimensional material layer.

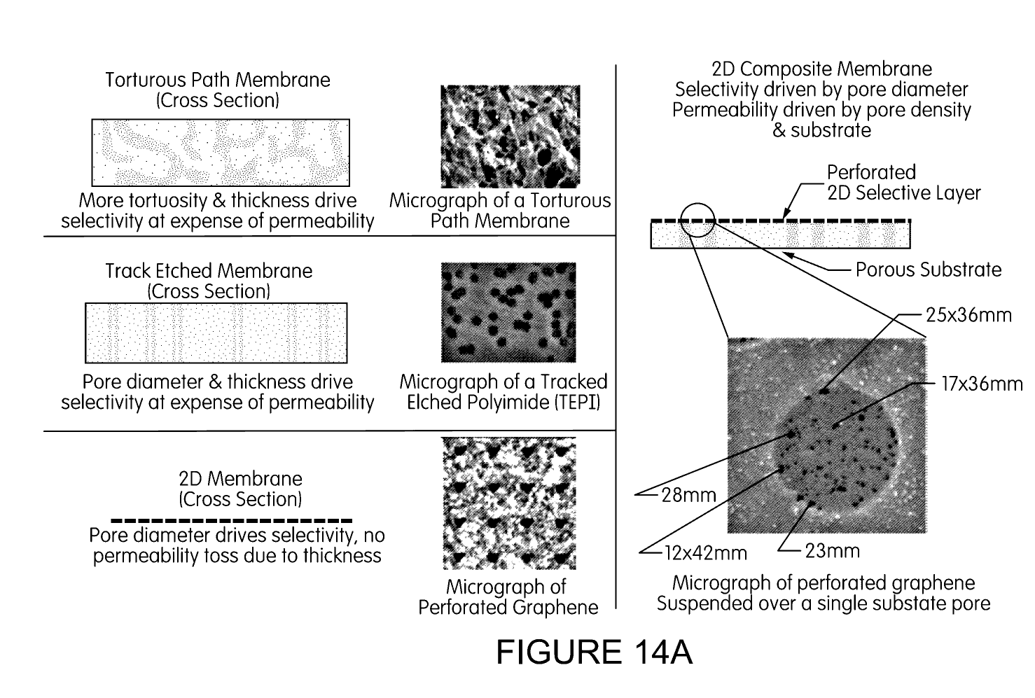

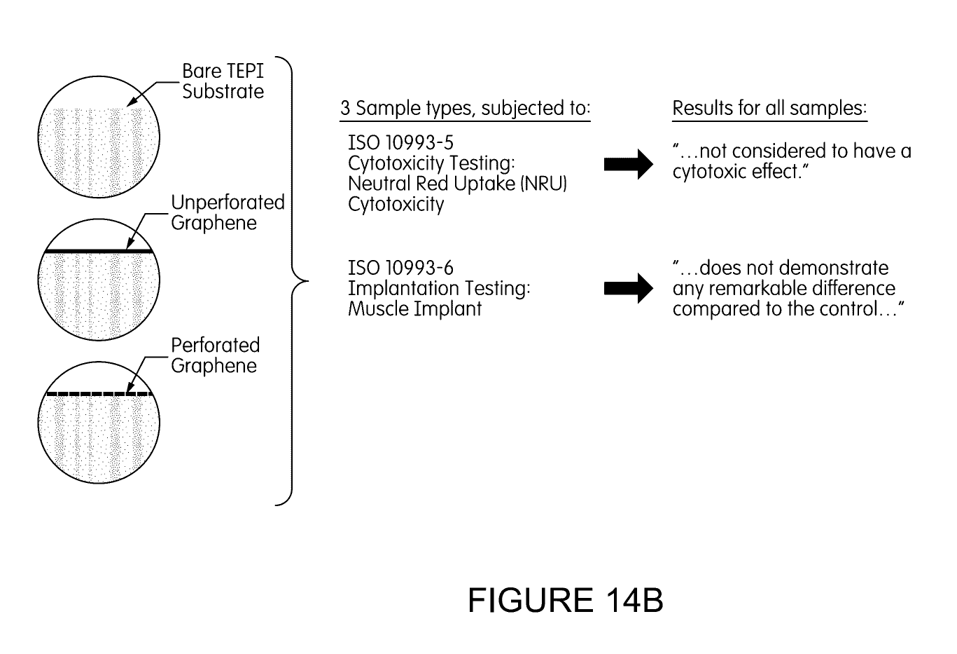

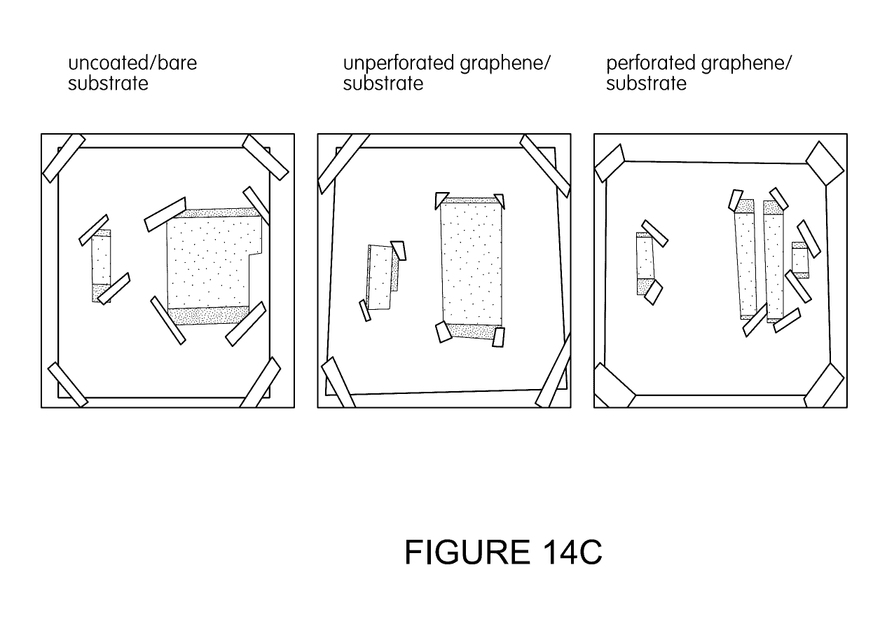

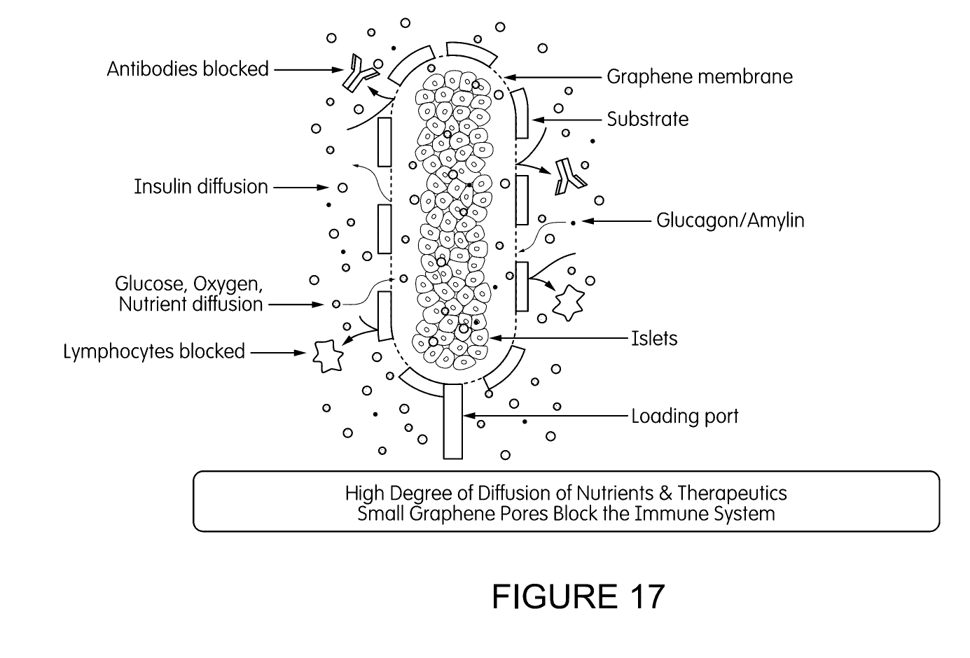

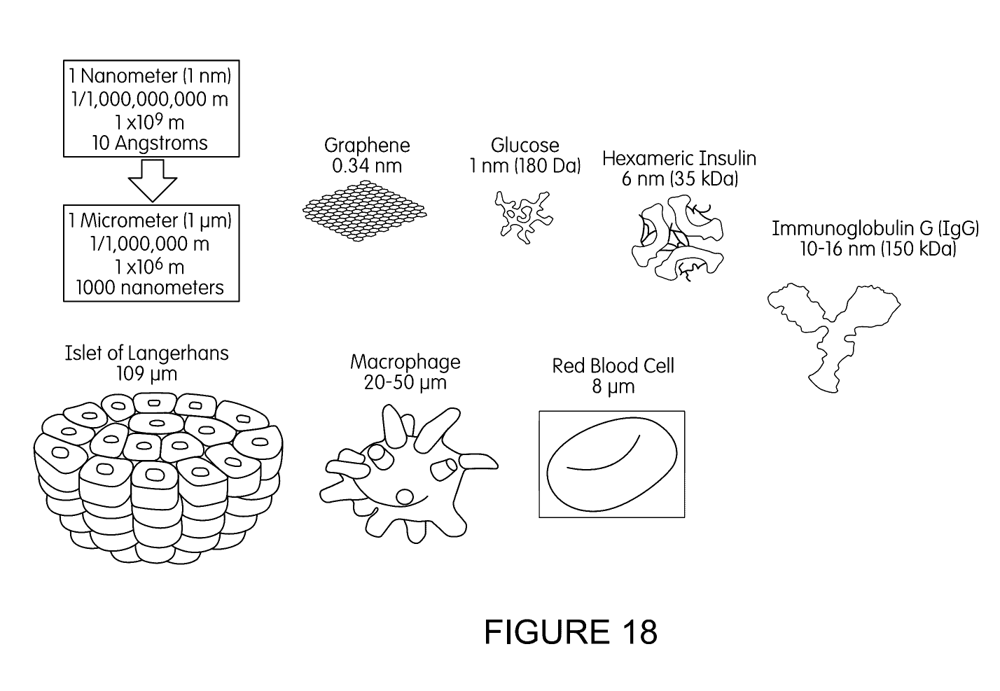

In some embodiments, a substrate layer is positioned on one or both sides or surfaces of the two-dimensional material. Thus, in some embodiments the substrate is positioned on the outside of the enclosure and in some cases is exposed to the external environment (see, e.g., FIG. 17, showing some embodiments with a device with a substrate positioned on the outside of an enclosure). In some embodiments, the substrate is positioned on the inside of the enclosure, and can be separated from an environment external to the enclosure (even though the substrate can be separated from the environment external to the enclosure, it can still be exposed to components from the external environment due to pores in the two-dimensional material layer and/or substrate layer). In some embodiments, the substrate is positioned on both the outside and the inside of the enclosure. In that case, the substrate on the outside of the enclosure can contain materials that are the same as or different from the substrate on the inside of the enclosure. In some embodiments, two or more substrate layers are positioned on the same side of the two-dimensional material layer (e.g., two or more substrate layers can be positioned on the outside of the enclosure). In some embodiments, the substrate is disposed directly on the two-dimensional material. In some embodiments, the substrate is disposed on the two-dimensional material with high conformance (e.g., by disposing a slightly wet substrate on the two-dimensional material). In some embodiments, the substrate is disclosed with low conformance. In some embodiments, the substrate is disposed indirectly on the two-dimensional material; for instance, an intermediate layer can be positioned between the substrate layer and the two-dimensional material layer. In some embodiments, the substrate layer is disposed directly or indirectly on another substrate layer. In some embodiments, the two-dimensional material is suspended on a substrate layer. In some embodiments, the substrate layer is affixed to the two-dimensional material layer (see, e.g., FIG. 14A, showing exemplary substrate layers, two-dimensional material layers, and substrate affixed to a two-dimensional material; see also FIG. 14B, showing some embodiments that were determined to be not cytotoxic based on cytotoxicity testing and implantation testing).

In some embodiments, the substrate layer can increase vascularization near the enclosure, thus prompting the formation of blood vessels and/or tissue ingrowth in close proximity to the enclosure. In some embodiments, the increased vascularization contributes to decreasing the effective distance between the blood stream and substances being eluted from the enclosure. In some embodiments, the increased vascularization contributes to viability of substances, such as cells, enclosed within the enclosure.

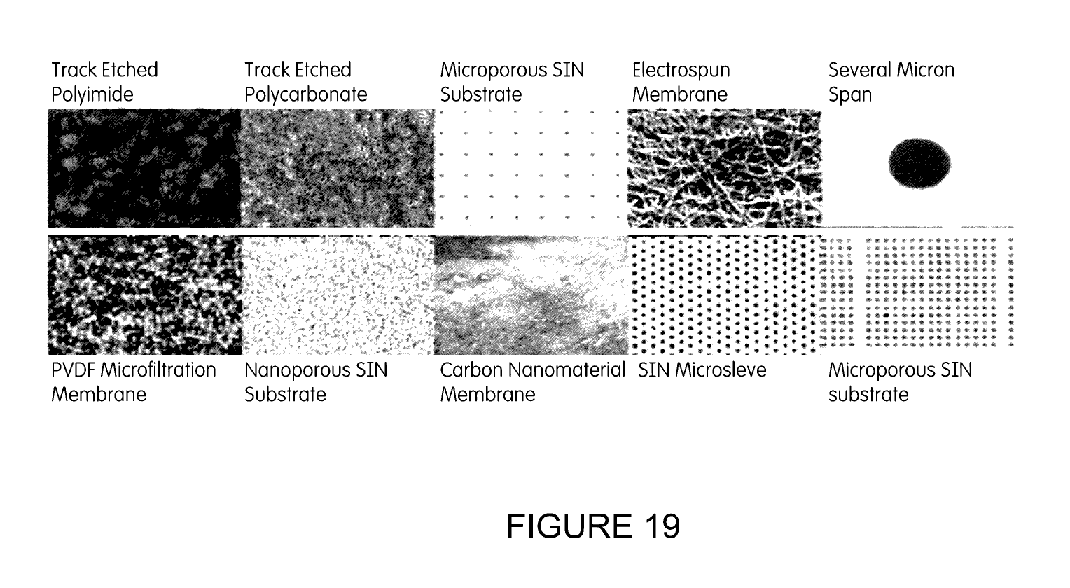

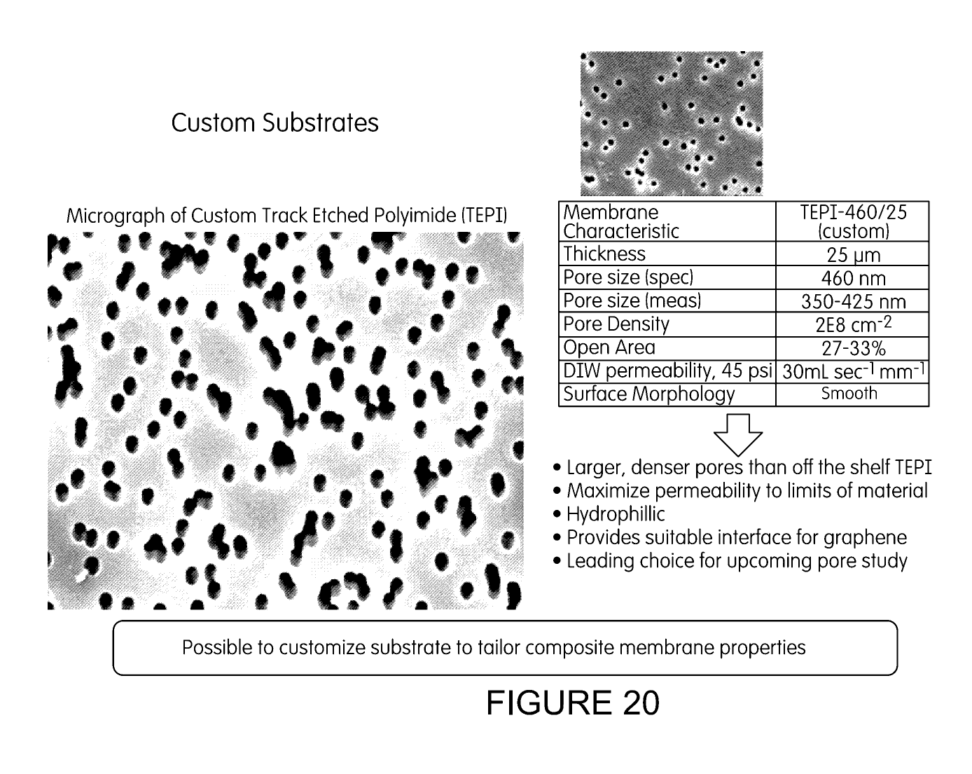

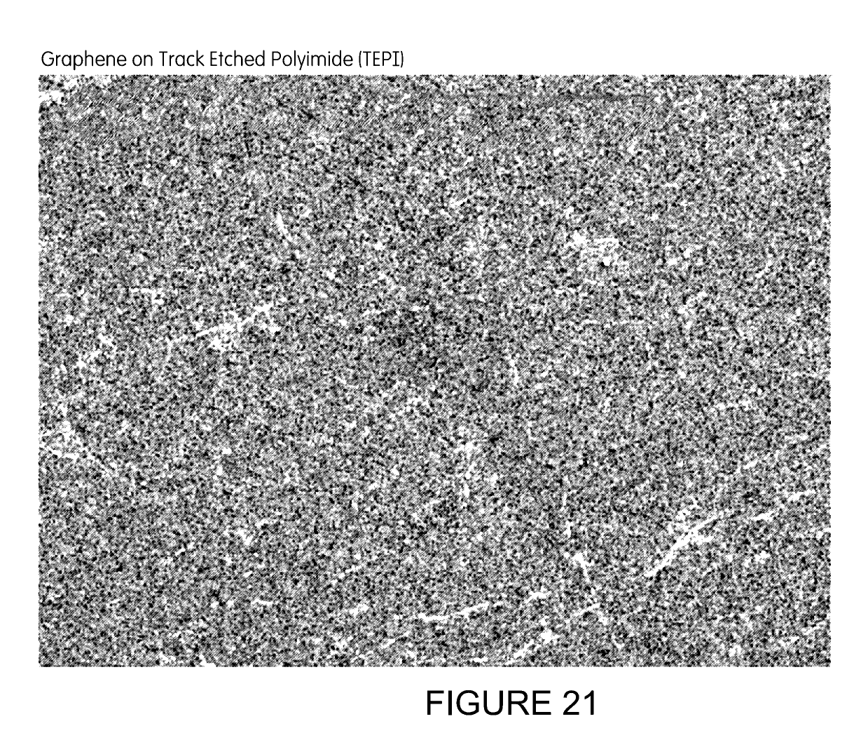

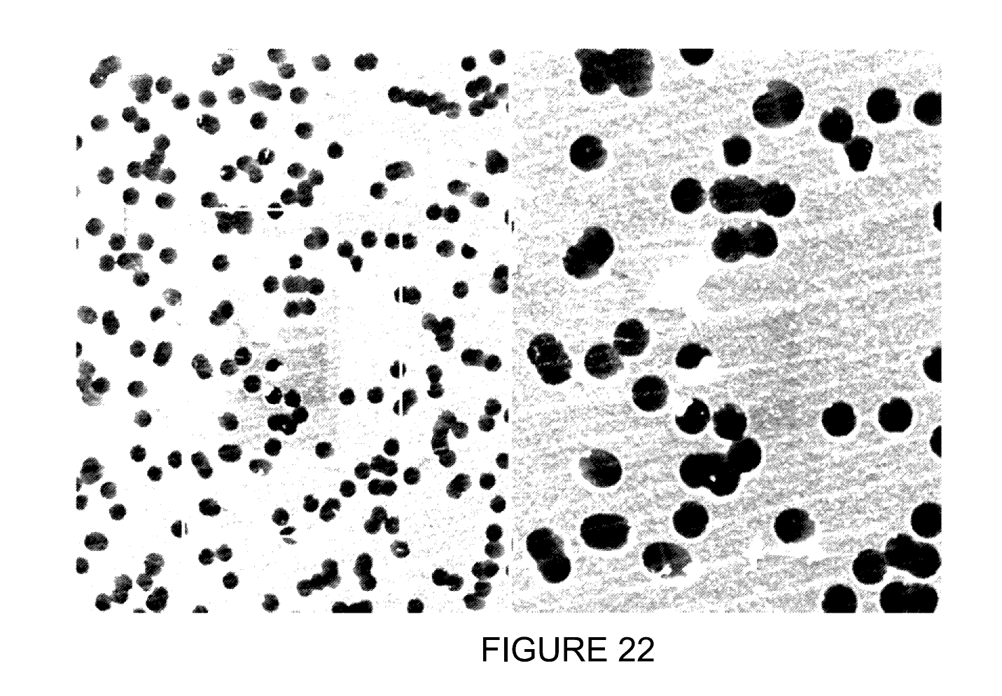

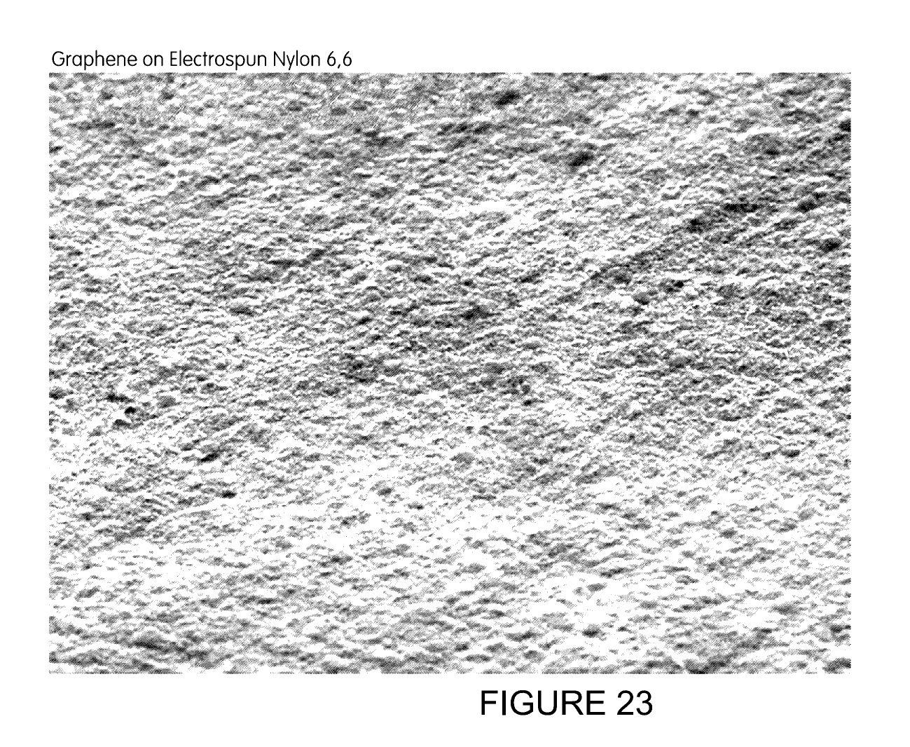

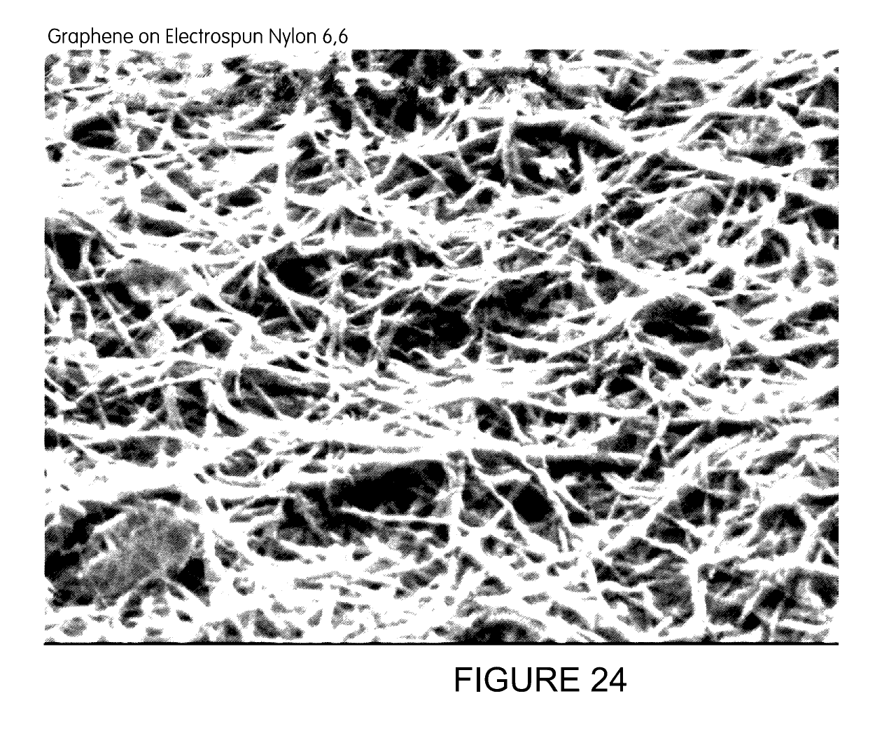

The substrate layer can be porous and/or nonporous. In some embodiments, the substrate layer contains porous and nonporous sections. In some embodiments the substrate layer comprises a porous or permeable fibrous layer. Porous substrates include, for example, one or more of ceramics and thin film polymers. Exemplary ceramics include nanoporous silica (silicon dioxide), silicon, SiN, and combinations thereof. In some embodiments, the substrate layer comprises track-etched polymers, expanded polymers, patterned polymers, woven polymers, and/or non-woven polymers. In some embodiments, the substrate layer comprises a plurality of polymer filaments. In some embodiments, the polymer filaments can comprise a thermopolymer, thermoplastic polymer, or melt polymer, e.g., that can be molded or set in an optional annealing step. In some embodiments, the polymer filaments are hydrophobic. In some embodiments, the polymer filaments are hydrophilic. In some embodiments, the substrate layer comprises a polymer selected from the group consisting of polysulfones, polyurethane, polymethylmethacrylate (PMMA), polyglycolid acid (PGA), polylactic acid (PLA), polyethylene glycol (PEG), polylactic-co-glycolic acid (PLGA), polyamides (such as nylon-6,6, supramid and nylamid), polyimides, polypropylene, polyethersulfones (PES), polyvinylidine fluoride (PVDF), cellulose acetate, polyethylene, polypropylene, polycarbonate, polytetrafluoroethylene (PTFE) (such as Teflon), polyvinylchloride (PVC), polyether ether ketone (PEEK), mixtures and block co-polymers of any of these, and combinations and/or mixtures thereof. In some embodiments, the polymers are biocompatible, bioinert and/or medical grade materials. By way of example, FIG. 19 shows some embodiments of graphene disposed on various different substrates. FIG. 20 shows micrographs some embodiments of custom track-etched polyimide. FIGS. 21 and 22 show micrographs of some embodiments of graphene disposed on track-etched polyimide. FIGS. 23 and 24 show micrographs of some embodiments of graphene disposed on electrospun nylon 6,6.

In some embodiments, the substrate layer comprises a biodegradable polymer. In some embodiments, a substrate layer forms a shell around the enclosure (e.g., it completely engulfs the enclosure). In some embodiments, the substrate layer shell, or a portion thereof, can be dissolved or degraded, e.g., in vitro. In some embodiments, the shell can be loaded with additives, including additives that protect substances inside the enclosure from air or prevent the need for a stabilizing agent.

Suitable techniques for depositing or forming a porous or permeable polymer on the two-dimensional material include casting or depositing a polymer solution onto the two-dimensional material or intermediate layer using a method such as spin-coating, spray coating, curtain coating, doctor-blading, immersion coating, electrospinning, or other similar techniques. Electrospinning techniques are described, e.g., in US 2009/0020921 and/or U.S. application Ser. No. 14/609,325, both of which are hereby incorporated by reference in their entirety.

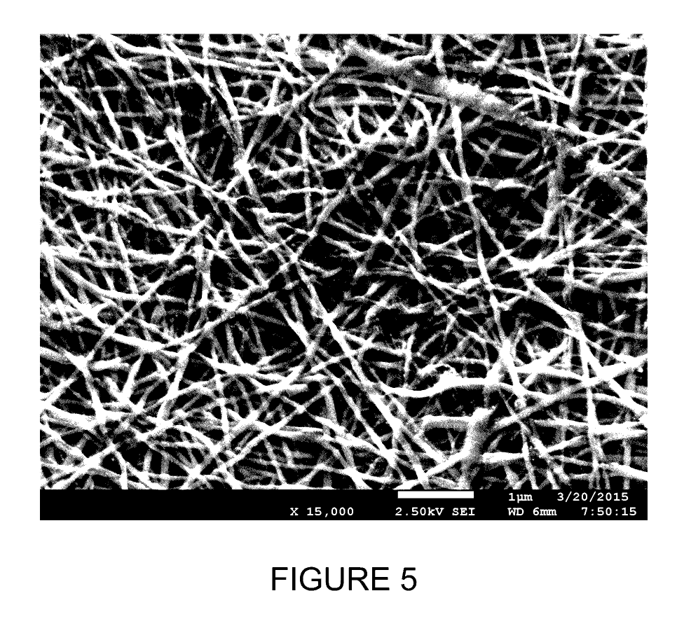

In some embodiments, the process for forming a substrate layer includes an electrospinning process in which a plurality of polymer filaments are laid down to form a porous mat, e.g., on the two-dimensional material layer. In some embodiments, the mat has pores or voids located between deposited filaments of the fibrous layer. FIG. 5 shows an illustrative SEM micrograph of a graphene or graphene-based film deposited upon a plurality of electrospun PVDF fibers. In some embodiments, the electrospinning process comprises a melt electrospinning process or a solution electrospinning process, such as a wet electrospinning process or a dry electrospinning process. (See, e.g., Sinha-Ray et al. J. Membrane Sci. 485, 1 Jul. 2015, 132-150.) In some embodiments, the polymer can be present in a spin dope at a concentration of 2 wt. % to 15 wt. %, or 5 wt. % to 10 wt. %, or about 7 wt. %. Suitable solvents for the spin dope include any solvent that dissolves the polymer to be deposited and which rapidly evaporates, such as m-cresol, formic acid, dimethyl sulfoxide (DMSO), ethanol, acetone, dimethylacetamide (DMAC), dimethylformamide (DMF), water, and combinations thereof. In some embodiments, the spin dope solvent is biocompatible and/or bioinert. In some embodiments, the amount of solvent used can influence the morphology of the substrate layer. In dry electrospinning processes, the spun fibers of the fibrous layer can remain as essentially discrete entities once deposited. In some embodiments, wet electrospinning processes deposit the spun fibers such that they are at least partially fused together when deposited. In some embodiments, the size and morphology of the deposited fiber mat (e.g., degree of porosity, effective pore size, thickness of fibrous layer, gradient porosity) can be tailored based on the electrospinning process used.

The porosity of the fibrous layer can include effective void space values (e.g. measured via imagery) up to about 95% (i.e., the layer is 95% open), about 90%, about 80%, or about 60%, with a broad range of void space sizes. In some embodiments, a single spinneret can be moved to lay down a mat of the fibrous layer. In some embodiments, multiple spinnerets can be used for this purpose. In some embodiments, the spun fibers in an electrospun fibrous layer can have a fiber diameter ranging from about 1 nm to about 100 μm, or about 10 nm to about 1 μm, or about 10 nm to about 500 nm, or about 100 nm to about 200 nm, or about 50 nm to about 120 nm, or about 1 μm to about 5 μm, or about 1 μm to about 6 μm, or about 5 μm to about 10 μm. In some embodiments, the fiber diameter is directly correlated with a depth (Z-axis) of a pore abutting the two-dimensional material (disposed in the X-Y plane), and large diameter fibers can lead to large unsupported spans of material.

In some embodiments, the substrate layer can have pores (e.g., void spaces) with an effective pore size of from about 1 nm to about 100 μm, or about 10 nm to about 1 μm, or about 10 nm to about 500 nm, or about 100 nm to about 200 nm, or about 50 nm to about 120 nm, or about 1 μm to about 5 μm, or about 1 μm to about 6 μm, or about 5 μm to about 10 μm. Pore diameters in the substrate layer can be measured, for example, via porometry methods (e.g., capillary flow porometry) or extrapolated via imagery.

In some embodiments, the substrate layer can have an average pore size gradient throughout its thickness. “Pore size gradient” describes a layer with a plurality of pores, where the average diameter of the pores increases or decreases based on the proximity of the pore to the two-dimensional material. For example, a fibrous layer can have an average pore size gradient that decreases nearer the surface of a graphene-based material. In some embodiments, an average pore size of the fibrous layer is smaller nearer the surface of the graphene-based material than at an opposite surface of the fibrous layer. For example, the fibrous layer can have effective pore diameters of less than about 200 nm close to the intermediate layer or the two-dimensional material layer which can increase to greater than 100 μm at the maximum distance away from the intermediate layer or two-dimensional material layer.

In some embodiments, the fibrous layer can have a “porosity gradient” throughout its thickness, which can be measured for instance using imagery. “Porosity gradient” describes a change, along a dimension of the fibrous layer, in the porosity or total pore volume as a function of distance from the two-dimensional material layer. For example, throughout the thickness of the porous fibrous layer, the porosity can change in a regular or irregular manner. A porosity gradient can decrease from one face of the fibrous layer to the other. For example, the lowest porosity in the fibrous layer can be located spatially closest to the two-dimensional material, and the highest porosity can be located farther away (e.g., spatially closer to an external environment). A porosity gradient of this type can be achieved by electrospinning fibers onto a two-dimensional material such that a fiber mat is denser near the surface of the two-dimensional material and less dense further from the surface of the two-dimensional material. In some embodiments, a substrate layer can have a relatively low porosity close to the two-dimensional material, a higher porosity at a mid-point of the fibrous layer thickness (which can, for example, contain a supporting mesh for reinforcement or other particles), and return to a relatively low porosity at an external surface distal to the two-dimensional material.

In some embodiments, the substrate layer can have a “permeability gradient” throughout its thickness. “Permeability gradient,” as used herein, describes a change, along a dimension of the fibrous layer, in the “permeability” or rate of flow of a liquid or gas through a porous material. For example, throughout the thickness of the fibrous layer, the permeability can change in a regular or irregular manner. A permeability gradient can decrease from one face of the fibrous layer to the other. For example, the lowest permeability in the fibrous layer can be located spatially closest to the graphene or graphene-based film or other two-dimensional material, and the highest permeability can be located farther away. Those of skill in the art will understand that permeability of a layer can increase or decrease without pore diameter or porosity changing, e.g., in response to chemical functionalization, applied pressure, voltage, or other factors.

In some embodiments, both the two-dimensional material layer and the substrate layer include a plurality of pores therein. In some embodiments, both the two-dimensional material and the substrate layer contain pores, and the pores in the two-dimensional material layer are smaller, on average, than the pores in the substrate layer. In some embodiments, the median pore size in the two dimensional material layer is smaller than the median pore size in the substrate layer. For example, in some embodiments, the substrate layer can contain pores with an average and/or median diameter of about 1 μm or larger and the two-dimensional material layer can contain pores with an average and/or median diameter of about 10 nm or smaller. Accordingly, in some embodiments, the average and/or median diameter of pores in the two-dimensional material layer is at least about 10-fold smaller than the average and/or median diameter of pores in the substrate layer. In some embodiments, the average and/or median diameter of pores in the two-dimensional material layer is at least about 100-fold smaller than are the average and/or media diameter of pores in the substrate layer.

Some embodiments comprise an enclosure with low or no toxicity, such as cytotoxicity. In some embodiments, the enclosure is not cytotoxic when implanted into a subject. In some embodiments, the enclosure is not cytotoxic to cells, skin, blood, bodily fluids, or muscle. In some embodiments, the enclosure is not cytotoxic when injected into a subject. In some embodiments, the enclosure is not cytotoxic when ingested by a subject. In some embodiments, the enclosure is not cytotoxic when used in vitro.

Some embodiments comprise a two-dimensional material (e.g., a graphene based material), such as a porous two-dimensional material, with low or no toxicity, such as cytotoxicity. In some embodiments, the two-dimensional material is not cytotoxic to cells, skin, blood, bodily fluids, or muscle. In some embodiments, the two-dimensional material is not cytotoxic when implanted into a subject. In some embodiments, the two-dimensional material is not cytotoxic when injected into a subject. In some embodiments, the two-dimensional material is not cytotoxic when ingested by a subject. In some embodiments, the two-dimensional material is not cytotoxic when used in vitro. In some embodiments, a two-dimensional material can be affixed to or disposed on a second material (e.g., a substrate) without substantially affecting the cytotoxicity of the second material. In some embodiments, affixing the two-dimensional material to (or disposing it on) the second material can reduce cytotoxicity of the second material.

Some embodiments comprise a composite structure with low or no toxicity, such as cytotoxicity. In some embodiments, the composite structure is not cytotoxic to cells, skin, blood, bodily fluids, or muscle. In some embodiments, the composite structure is not cytotoxic when implanted into a subject. In some embodiments, the composite structure is not cytotoxic when injected into a subject. In some embodiments, the composite structure is not cytotoxic when ingested by a subject. In some composite structure is not cytotoxic when used in vitro.

Cytotoxicity can be measured, for instance, using cell viability assays or implantation testing. In some embodiments, greater than about 70% of cells exposed to the enclosure and/or composite structure remain viable at least 24 hours after exposure. In some embodiments, at least about 80%, at least about 85%, at least about 90%, at least about 95%, at least about 98%, or at least about 99% of cells exposed to the enclosure and/or composite structure remain viable at least 24 hours after exposure.

In some embodiments, the device, enclosure and/or composite material has a bioreactivity rating of about 8.9 or less, such as from about 3.0 to about 8.9, or about 0.0 to about 2.9. In some embodiments, the device, enclosure and/or composite material has a bioreactivity rating of about 0.0, about 0.5, about 0.7, about 1.0, about 1.5, about 2.0, about 2.2, about 2.5, or about 2.9.

In some embodiments, tissue surrounding an implanted enclosure and/or composite structure do not exhibit substantial signs of cytotoxicity. Thus, in some embodiments, the enclosure and/or composite structure causes no, mild, or moderate signs of inflammation, encapsulation, hemorrhage, necrosis, discoloration, polymorphonuclear cells, lymphocytes, plasma cells, macrophages, giant cells, necrosis, neovascularization, fibrosis, fatty infiltrate, or combinations thereof in tissues exposed to the enclosure and/or composite structure. In some embodiments, macroscopic evaluation of tissue exposed to the enclosure and/or composite structure reveals no signs of inflammation, encapsulation, hemorrhage, necrosis, discoloration, or combinations thereof. In some embodiments, macroscopic evaluation of tissue exposed to the enclosure and/or composite structure reveals mild or moderate signs of inflammation, encapsulation, hemorrhage, necrosis, discoloration, or combinations thereof.

In some embodiments, microscopic evaluation of tissue exposed to the enclosure and/or composite structure reveals no signs an inflammatory response, such as signs of polymorphonuclear cells, lymphocytes, plasma cells, macrophages, giant cells, necrosis, or combinations thereof. In some embodiments, microscopic evaluation of tissue exposed to the enclosure and/or composite structure reveals minimal or mild signs an inflammatory response, such as signs of polymorphonuclear cells, lymphocytes, plasma cells, macrophages, giant cells, necrosis, or combinations thereof. In some embodiments, microscopic evaluation of tissue exposed to the enclosure and/or composite structure reveals no signs a healing response, such as neovascularization, fibrosis, fatty infiltrate, or combinations thereof. In some embodiments, microscopic evaluation of tissue exposed to the enclosure and/or composite structure reveals minimal or mild signs a healing response, such as neovascularization, fibrosis, fatty infiltrate, or combinations thereof.

In some embodiments, extent of cytotoxicity is classified based on macroscopic or microscopic evaluation, and classification can be relative to cytotoxicity of a control enclosure and/or structure. Thus, in some embodiments no, mild, or moderate signs of inflammation, encapsulation, hemorrhage, necrosis, discoloration, polymorphonuclear cells, lymphocytes, plasma cells, macrophages, giant cells, necrosis, neovascularization, fibrosis, fatty infiltrate, or combinations thereof are as compared to a control (e.g., in some embodiments, the enclosure and/or composite structure has no signs of inflammation if observed inflammation is less than is observed using a control).

Some embodiments comprise methods of releasing a substance into an environment from an enclosure with low or no toxicity (e.g., cytotoxicity) to the environment. Some embodiments comprise treating a condition or disease, such as diabetes, by an enclosure with low or no cytotoxicity into the subject. Some embodiments comprise using the non-cytotoxic or low-cytotoxic enclosure in methods of immunoisolation (i.e., protecting substances from an immune reaction), timed drug release (e.g., sustained or delayed release), hemodialysis, or hemofiltration.

Some embodiments comprise encapsulating a device with a composite structure comprising (a) a perforated graphene-based material layer and (b) a substrate layer affixed directly or indirectly to at least one surface of the perforated graphene-based material. In some embodiments, the encapsulated device has a reduced cytotoxicity as compared to a comparable device without a perforated graphene-based material layer.

Some embodiments comprise methods of coating a therapeutic device with the composite structure. In some embodiments, the composite structure is applied to the exterior of the therapeutic device. Some embodiments comprise the coated therapeutic device. In some embodiments, the coated therapeutic device has a lower toxicity (e.g., cytotoxicity) than a comparable therapeutic device that is not coated with the composite structure.

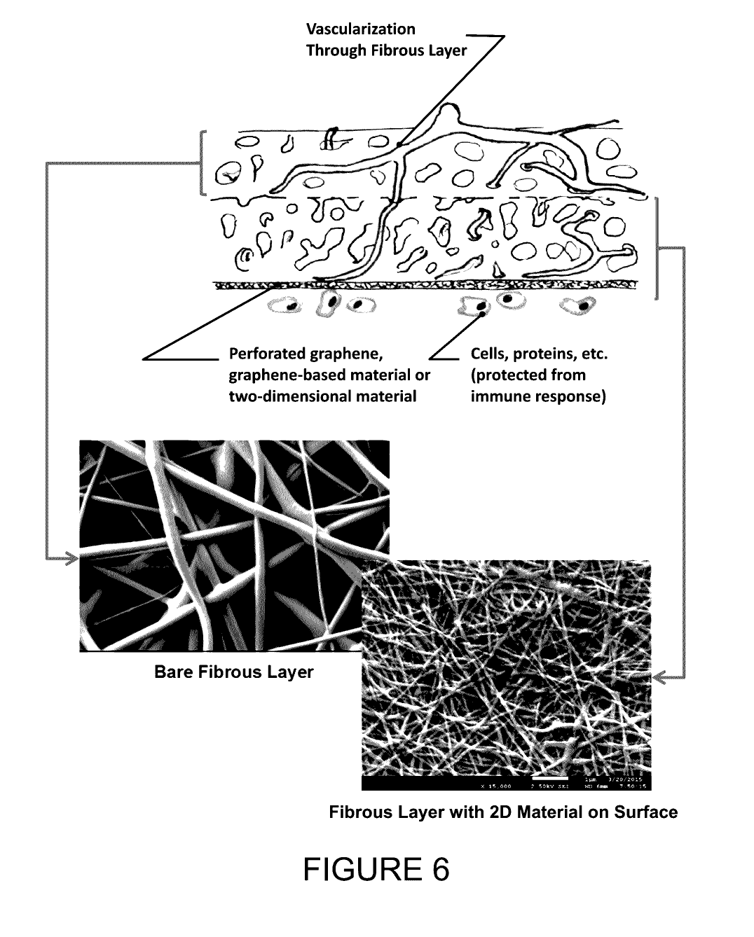

FIG. 6 illustrates a portion of an enclosure in a biological environment in contact with biological tissue in which an enclosure comprises one or more substrate layers, such as fibrous layers positioned on the outside of the perforated two-dimensional material. FIG. 6 also shows capillary vascularization into the substrate layer. Without being bound by theory, it is believed that the biocompatibility of graphene can further promote this application, particularly by functionalizing the graphene to improve compatibility with a particular biological environment (e.g., via available edge bonds, bulk surface functionalization, pi-bonding, and the like). Functionalization can provide enclosures having added complexity for use in treating local and systemic disease. FIG. 6 also shows a wall of an enclosure with a perforated two-dimensional material having hole sizes in a range that will retain cells. The external biological environment abutting the enclosure (the full enclosure is not shown) in FIG. 6 is separated from cells, proteins, etc., positioned inside the enclosure. As illustrated, in some embodiments implantation of such an enclosure contemplates vascularization into a substrate layer positioned on the outside of the enclosure.

In some embodiments, the substrate layer can provide a scaffold for tissue growth, cell growth and/or vascularization. In some embodiments, the substrate layer or wall comprises additives, such as pharmaceuticals, cells, growth factors (e.g., VEGF), signaling molecules, cytokines, clotting factors, blood thinners, immunosuppressants, antimicrobial agents, hormones, antibodies, minerals, nutrients or combinations thereof. In some embodiments, additives such as pharmaceuticals, cells, growth factors, clotting factors, blood thinners, immunosuppressants, antimicrobial agents, hormones, antibodies, antigens (e.g., IgG-binding antigens) or an antibody-binding fragment thereof, minerals, nutrients or combinations thereof are positioned on the inside of the disclosure. In some embodiments, the substrate layer or wall comprises materials toxic to bacteria or cells (without being bound by theory, it is believed that incorporating toxic materials into the wall will prevent passage of potentially dangerous or detrimental cells across the wall).

In some embodiments, additives beneficially promote cell or tissue viability or growth, reduce or prevent infection, improve vascularization to or near the enclosure, improve biocompatibility, reduce biofouling, and/or reduce the risk of adverse reactions. In some embodiments, additives can modulate properties, such as hydrophobicity or hydrophilicity, of the substrate layer. In some embodiments, additives can be used to modulate elution of a substance from a compartment in the enclosure. For instance, additives can confer shell-like properties to a substrate layer, such that degradation or removal of the additives allows substances in the compartment to escape the enclosure (and, by extension, substances from the external environment to enter to enclosure).

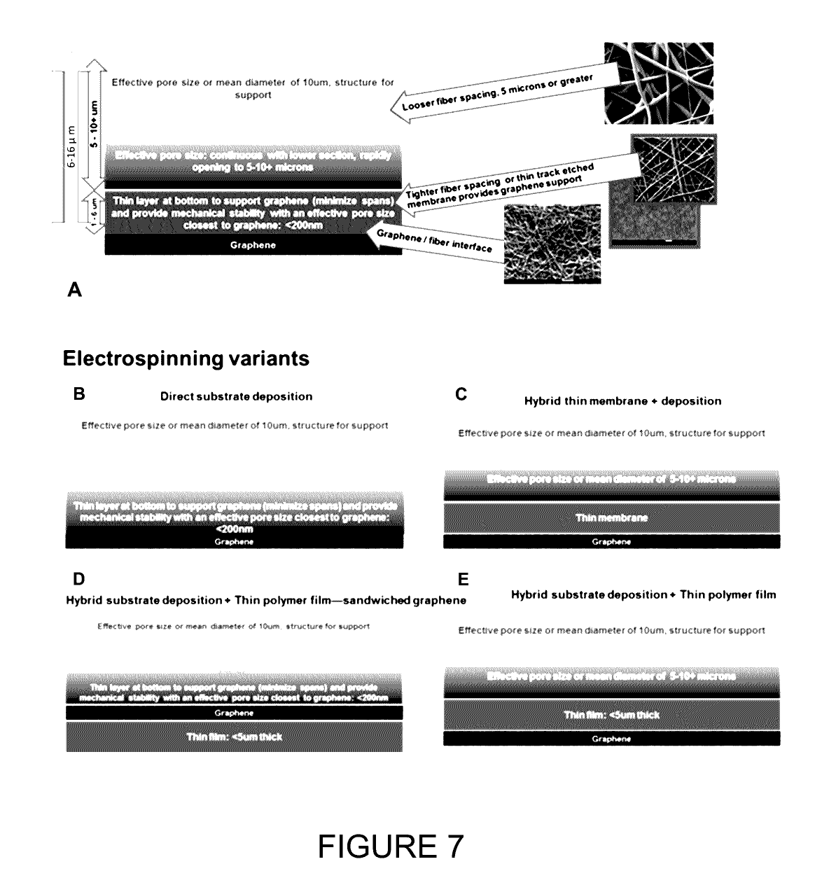

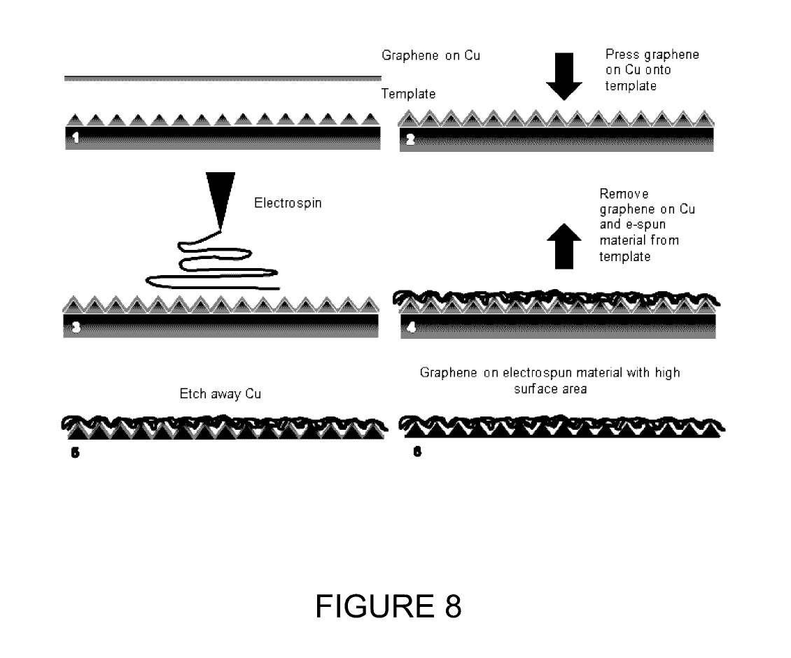

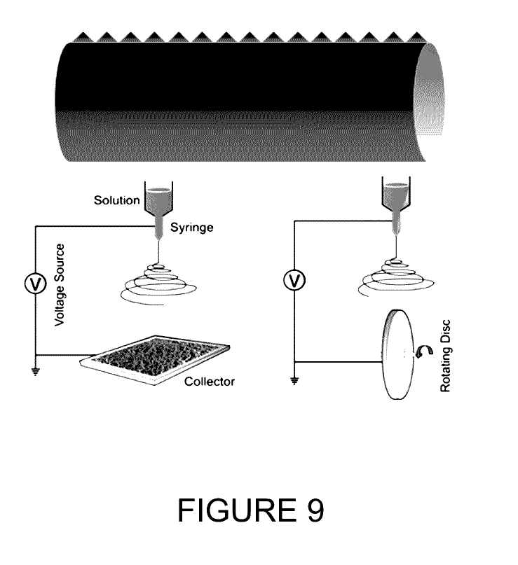

Some embodiments comprise a composited structure that include a two-dimensional material layer and a substrate layer. In some embodiments, a composite structure includes a support material (see, e.g., FIG. 7D) disposed on an opposite side of the two-dimensional material from the substrate layer. In some embodiments, a composite structure comprises an intermediate layer between the two-dimensional material and the substrate layer, e.g., as shown in FIGS. 7A, 7C and 7E. FIG. 7 shows schematic illustrations of composite structures comprising two-dimensional materials (e.g., graphene), an optional intermediate layer (e.g., track etched polymer membrane), and a fibrous layer having a tighter fiber spacing nearer the two-dimensional material and an increasing effective pore size further from the two-dimensional material. FIG. 7A shows SEM micrographs of the fibrous material with (bottom two expanded micrographs) and without (top two expanded micrographs) the two-dimensional material on the surface of the fibrous material. FIG. 7A also shows SEM micrographs of high fiber density (bottom), medium fiber density (middle) and low fiber density (top) substrates.

In some embodiments, the intermediate layer promotes adhesion between the two-dimensional material layer and the substrate layer. Thus, in some embodiments, the enclosure comprises an intermediate layer disposed between the two-dimensional material layer and the substrate layer. In some embodiments, the enclosure comprises an intermediate layer positioned between two substrate layers on the same side of the two-dimensional material layer.

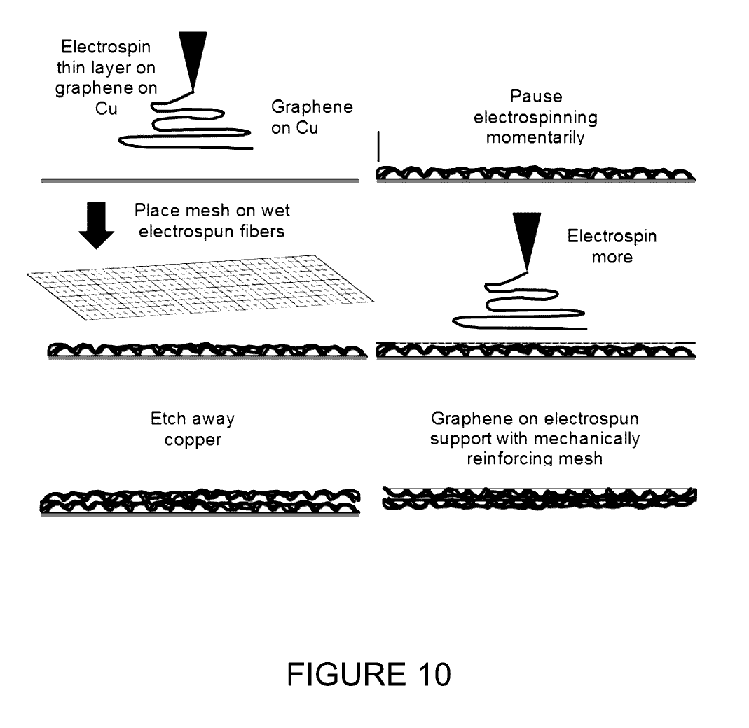

In some embodiments, the intermediate layer comprises carbon nanotubes, lacey carbon, nanoparticles, lithographically patterned low-dimensional materials, silicon and silicon nitride micromachined material, a fine mesh, such as a transmission electron microscopy grid, or combinations of these. FIG. 10 shows an illustrative schematic of a process for manufacturing a two-dimensional material on a fibrous layer with mesh reinforcement. In some embodiments, the intermediate layer can be a thin, smooth, porous polymer layer, such as a track etched polymer. In some embodiments, the intermediate layer has a thickness of from 3 nm to 10 μm, 10 nm to 10 μm, 50 nm to 10 μm, 100 nm to 10 μm, 500 nm to 10 μm, 1 μm to 10 μm, or 2 μm to 6 μm. In some embodiments, the composite structure has a thickness of from 1 μm to 100 μm, 2 μm to 75 μm, 3 μm to 50 μm, 4 μm to 40 μm, 5 μm to 30 μm, 6 μm to 25 μm, or 6 μm to 20 μm, or 6 μm to 16 μm.

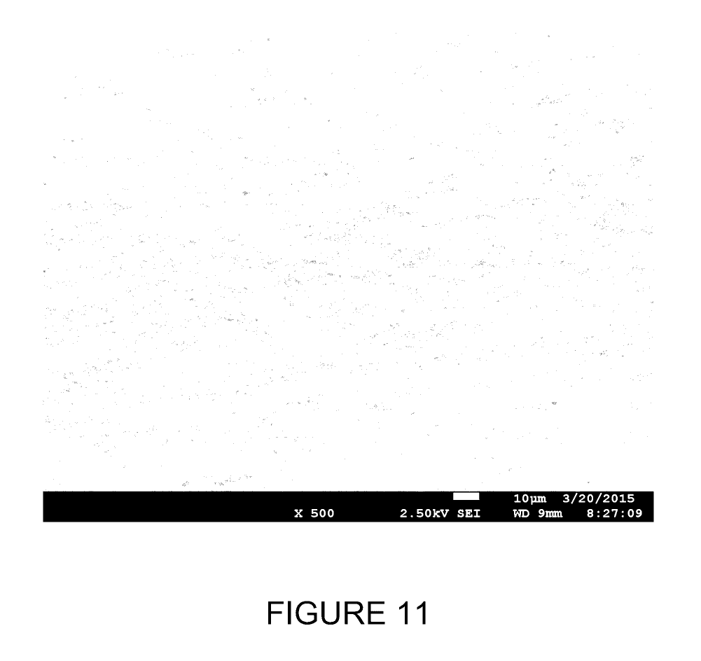

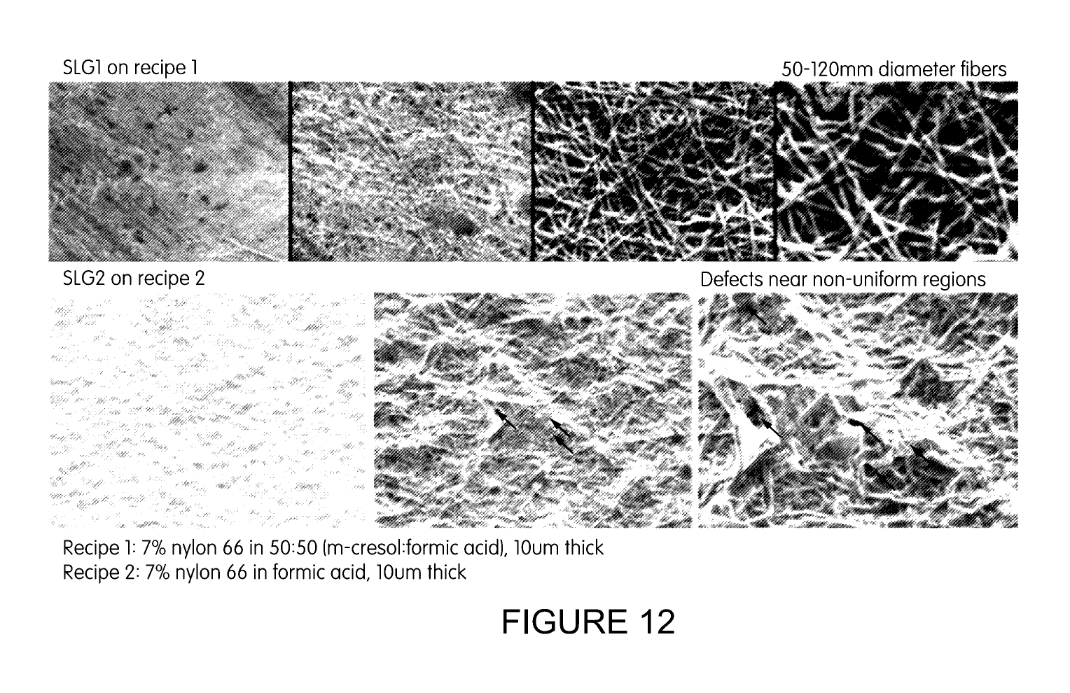

In some embodiments, an enclosure or composite structure includes a fibrous layer affixed to multiple sheets of graphene or graphene-based material. In some embodiments, the sheets of graphene or graphene-based materials are stacked upon one another with one of the sheets affixed directly or indirectly to the fibrous layer. FIG. 11 shows an illustrative SEM micrograph of two layers of graphene or graphene-based material on a fibrous layer. In some embodiments, one or more sheets of graphene or graphene-based material can be affixed to a first surface of a fibrous layer and one or more sheets of graphene or graphene-based material can be affixed to a second surface of the fibrous layer. In some embodiments, the graphene-based material is applied to a fully-formed substrate layer, such as a fully-formed electrospun substrate layer. Some embodiments comprise putting multiple layers of the graphene-based material onto the substrate layer (e.g., the fully-formed substrate layer). Without being bound by theory, it is believed that adding multiple layers of graphene-based material onto the substrate layer allows complete coverage of the substrate layer with the graphene-based material.

In some embodiments, the enclosure comprises a single compartment that does not contain sub-compartments. In some embodiments, the single compartment is in fluid communication with an external environment separated from the compartment, e.g., by a wall. In some embodiments, the enclosure has a plurality of sub-compartments. In some embodiments, the sub-compartments are in fluid communication with an environment outside the sub-compartment. In some embodiments, each sub-compartment comprises a wall that allows passage of one or more substances into and/or out of the sub-compartment. In some embodiments, the wall or a portion thereof comprises a perforated two-dimensional material, a polymer, a hydrogel, or some other means of allowing passage of one or more substance into and/or out of the sub-compartment. In some embodiments, an enclosure is subdivided into two sub-compartments separated from each other at least in part by perforated two-dimensional material, such that the two sub-compartments are in direct fluid communication with each other through holes in the two-dimensional material. In some embodiments, the enclosure is subdivided into two sub-compartments each comprising two-dimensional material which sub-compartments are in direct fluid communication with each other through holes in the two-dimensional material and only one of the sub-compartments is in direct fluid communication with an environment external to the enclosure. In some embodiments, the enclosure is subdivided into two sub-compartments each comprising two-dimensional material which sub-compartments are in direct fluid communication with each other through holes in the two-dimensional material and both of the sub-compartments are also in direct fluid communication with an environment external to the enclosure.

In some embodiments, the enclosure has an inner sub-compartment and an outer sub-compartment each comprising a perforated two-dimensional material, wherein the inner sub-compartment is entirely enclosed within the outer sub-compartment, the inner and outer compartments are in direct fluid communication with each other through holes in the two-dimensional material and the inner sub-compartment is not in direct fluid communication with an environment external to the enclosure.

In some embodiments, where an enclosure has a plurality of sub-compartments each comprising a two-dimensional material, the sub-compartments are nested one within the other, each of which sub-compartments is in direct fluid communication through holes in two-dimensional material with the sub-compartment(s) to which it is adjacent, the outermost sub-compartment in direct fluid communication with an environment external to the enclosure, the remaining plurality of sub-compartments not in direct fluid communication with an environment external to the enclosure.

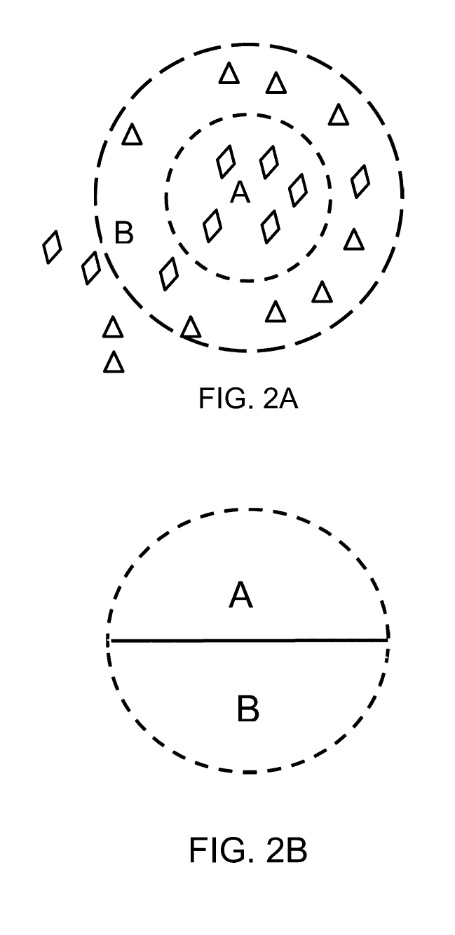

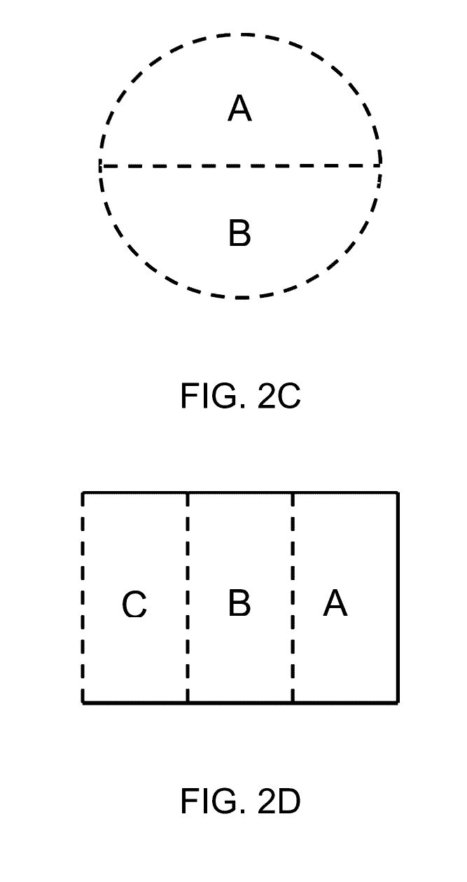

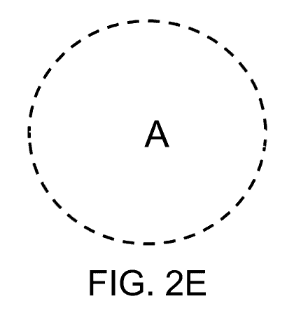

In some embodiments, a sub-compartment can have any shape or size. In some embodiments, 2 or 3 sub-compartments are present. Several examples of enclosure sub-compartments are illustrated in FIGS. 2A-2E. In FIG. 2A, a nested configuration is illustrated, such that sub-compartment B completely contains a smaller sub-compartment A, and substances in the centermost enclosure A can pass into the main enclosure B, and potentially react with or within the main compartment during ingress and egress therefrom. In this embodiment, one or more substances in A can pass into B and one or more substances in A can be retained in A and not enter B. Two sub-compartments in which one or more substances can pass directly between the sub-compartments are said to be in direct fluid communication. Passage between sub-compartments and between the enclosure and the external environment can be via holes of a perforated two-dimensional material. In some embodiments, the barrier (e.g., a membrane) between compartment A and B can be permeable to all substances in A or to certain substances in A (i.e., selective permeability). In some embodiments, the barrier between B and the external environment can be permeable to all substances in B or selectively permeable to certain substances in B. In FIG. 2A, sub-compartment A is in direct fluid communication with sub-compartment B which in turn is in direct fluid communication with the external environment. Compartment A in this nested configuration is in indirect fluid communication with the external environment via intermediate passage into sub-compartment B. The two-dimensional materials employed in different sub-compartments of an enclosure can be the same or different materials and the perforations or hole sizes in the two-dimensional material of different sub-compartments can be the same or different.

In FIG. 2B, the enclosure is bisected with an impermeable wall (e.g., formed of non-porous or non-permeable sealant) forming sub-compartments A and B, such that both sections have access to the egress location independently, but there is no direct or indirect passage of substances from A to B. (It will be appreciated, however, that substances exiting A or B can enter the other sub-compartment indirectly via the external environment.)

In FIG. 2C, the main enclosure is again bisected into sub-compartments A and B, but with a perforated material forming the barrier between the sub-compartments. Both sub-compartments not only have access to the egress location independently, but also can interact with one another, i.e. the sub-compartments are in direct fluid communication. In some embodiments, the barrier between sub-compartments A and B is selectively permeable, for example allowing at least one substance in A to pass into B, but not allowing the substances originating in B to pass to A. The porosity of the barrier between sub-compartments (e.g., sub-compartments A and B) can be the same as or different than the porosity of the sub-compartment walls in direct fluid communication with an environment external to the enclosure.

FIG. 2D illustrates an enclosure having three compartments. The enclosure is illustrated with sub-compartment A being in fluid communication with sub-compartment B, which in turn is in fluid communication with sub-compartment C, which in turn is in fluid communication with the external environment. Compartments A and B are not in fluid communication with the external environment, i.e. they are not in direct fluid communication with the external environment. Adjacent sub-compartments A and B and adjacent sub-compartments B and C are each separated by a perforated two-dimensional material and are thus in direct fluid communication with each other. Sub-compartment A is only in indirect fluid communication with compartment C and the external environment via sub-compartment B or B and C, respectively. Various other combinations of semi-permeable barrier or non-permeable barriers can be employed to separate sub-compartments. Various perforation size constraints can change depending on how the enclosure is ultimately configured. Regardless of the chosen configuration, in some embodiments the boundaries, or at least a portion thereof, of the enclosure can be constructed from a two-dimensional material such that the thickness of the two-dimensional material is less than the diameter of the substance to be passed selectively across the two-dimensional material.

FIG. 2E illustrates an enclosure having a single compartment (A) and no sub-compartments. In the Figure, the compartment is in direct fluid communication with an environment external to the enclosure.

In some embodiments, the presence of two or more sub-compartments containing the same substance(s) provides redundancy in function so that an enclosure can remain at least partially operable so long as at least one sub-compartment is not compromised.

The multiple physical embodiments for the enclosures and their uses can allow for various levels of interaction and scaled complexity of problems to be solved. For example, a single enclosure can provide drug elution for a given time period, or there can be multiple sizes of perforations to restrict or allow movement of distinct substances, each having a particular size.

Added complexity of the embodiments described herein with multiple sub-compartments can allow for interaction between compounds to catalyze or activate a secondary response (i.e., a “sense-response” paradigm). For example, if there are two sections of an enclosure that function independently, exemplary compound A can undergo a constant diffusion into the body, or either after a given time or in the presence of a stimulus from the body. In some embodiments, exemplary compound A can activate exemplary compound B, or inactivate functionalization that otherwise blocks exemplary compound B from escaping. In some embodiments, binding interactions to produce the foregoing effects can be reversible or irreversible. In some embodiments, exemplary compound A can interact with chemical cascades produced outside the enclosure, and a metabolite subsequent to the interaction can release exemplary compound B (e.g., by inactivating functionalization). Further examples utilizing effects that take place in a similar manner include using source cells (e.g., non-host; allogenic; xenogenic; autogenic; cadeaveric; stem cells, such as fully or partially differentiated stem cells) contained in an enclosure, within which secretions from the cell can produce a “sense-response” paradigm. In some embodiments, the presence of graphene in the “sense-response” paradigm does not hinder diffusion, thus allowing a fast time response as compared to enclosures that to not contain graphene.

In some embodiments, growth factors or hormones can be loaded in the enclosure to encourage vascularization (see FIG. 6). In some embodiments, survival of cells within the enclosure can be improved as a result of bi-directional passage of nutrients and waste into and out of the enclosure.

In some embodiments, the relative thinness of graphene can enable bi-directional passage across a wall (or portion thereof) of the enclosure in close proximity to blood vessels, particularly capillary blood vessels, and other cells. In some embodiments, using a graphene-based enclosure can provide differentiation over other solutions accomplishing the same effect because the graphene does not appreciably limit permeability; instead, the diffusion of molecules through the graphene apertures can limit the movement of a substance across the wall.

In some embodiments, the perforations allow for zeroth order diffusion through the wall. In some embodiments, osmotic pumps can be used to transport substances across the wall. In some embodiments, natural delta pressures in the body influence passage of substances across the wall. In some embodiments, convective pressure influences passage of substances across the wall. In some embodiments, it is possible to achieve high throughput flux through the wall of the enclosure.

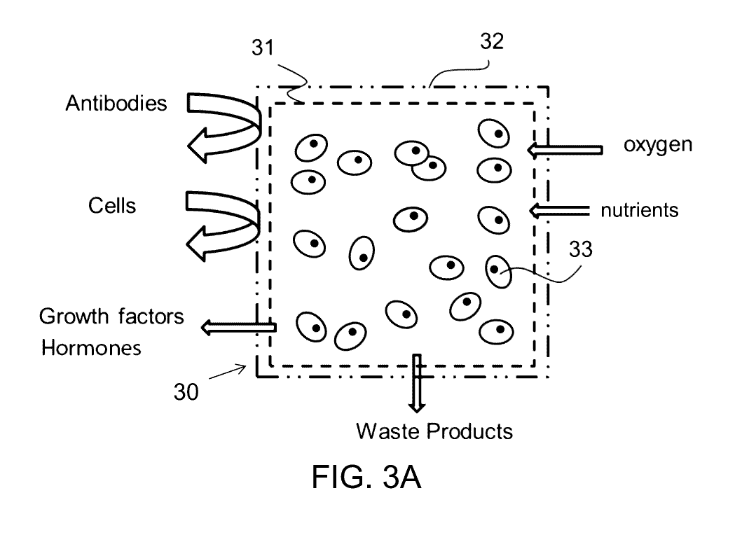

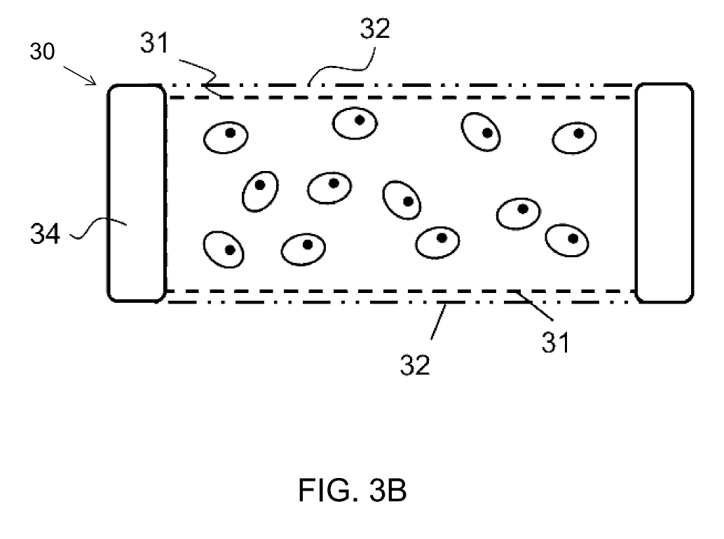

FIGS. 3A and 3B provide a schematic illustration of enclosures with a single compartment for immunoisolation (it will be appreciated that the enclosure can having a plurality of sub-compartments, for example, two or three sub-compartments). The enclosure (30) of FIG. 3A is shown as a cross-section formed by an inner sheet or layer (31) comprising perforated two-dimensional material, such as a graphene-based material, and an outer sheet or layer (32) of a substrate material (though in some embodiments, the inner layer comprises the substrate material, and the outer layer comprises the perforate two-dimensional material). The substrate material can be porous, selectively permeable or non-porous, and/or and non-permeable. However at least a portion of the support material is porous or selectively permeable. The enclosures in FIG. 3 contain selected living cells (33). FIG. 3B provides an alternative cross-section of the enclosure of FIG. 3A, showing the space or cavity formed between a first composite layer (32/31) and a second composite layer (32/31) (in the figure, the cavity is depicted to contain roughly circular symbols, which can be, e.g., cells or any other substance) where a sealant 34 is illustrated as sealing the edges of the composite layers. It will be appreciated that seals at the edges of the composite layers can be formed employing physical methods, such as clamping, crimping, or with adhesives. Methods and materials for forming the seals at the edges are not particularly limiting. In some embodiments, the sealing material provides a non-porous and non-permeable seal or closure. In some embodiments, a portion of the enclosure is formed from a sealant, such as a silicone, epoxy, polyurethane or similar material. In some embodiments, the sealant is biocompatible. For instance, in some embodiments the seal does not span the entire length or width of the device. In some embodiments, the seal forms a complete loop around the cavity. In some embodiments, the seal is formed as a frame at a perimeter of a two-dimensional material. In some embodiments, the seal is positioned, at least in part, interior to a perimeter of a two-dimensional material.

Some embodiments include methods for using graphene-based materials and/or other two-dimensional materials to transport, transfer, deliver, and/or allow passage of substances in or to a biological environment. Some embodiments comprise delivering substances to an environment external to the enclosure (e.g., a biological environment). In some embodiments, the substance positioned on the inside of the enclosure comprises one or more of atoms, molecules, viruses, bacteria, cells, particles and aggregates thereof. For example, the substance can include biological molecules, such as proteins, peptides, (e.g., insulin), nucleic acids, DNA, and/or RNA; pharmaceuticals; drugs; medicaments; therapeutics, including biologics and small molecule drugs; and combinations thereof.

If cells are placed within the enclosure, at least a portion of the enclosure can be permeable to oxygen and nutrients sufficient for cell growth and maintenance, to waste produced by the cell (e.g., CO₂), and/or to metabolites produced by the cell (e.g., insulin). In some embodiments, at least a portion of the enclosure is permeable to signaling molecules, such as glucose. In some embodiments, at least a portion of the enclosure is permeable to growth factors produced by cells, such as VEGF.

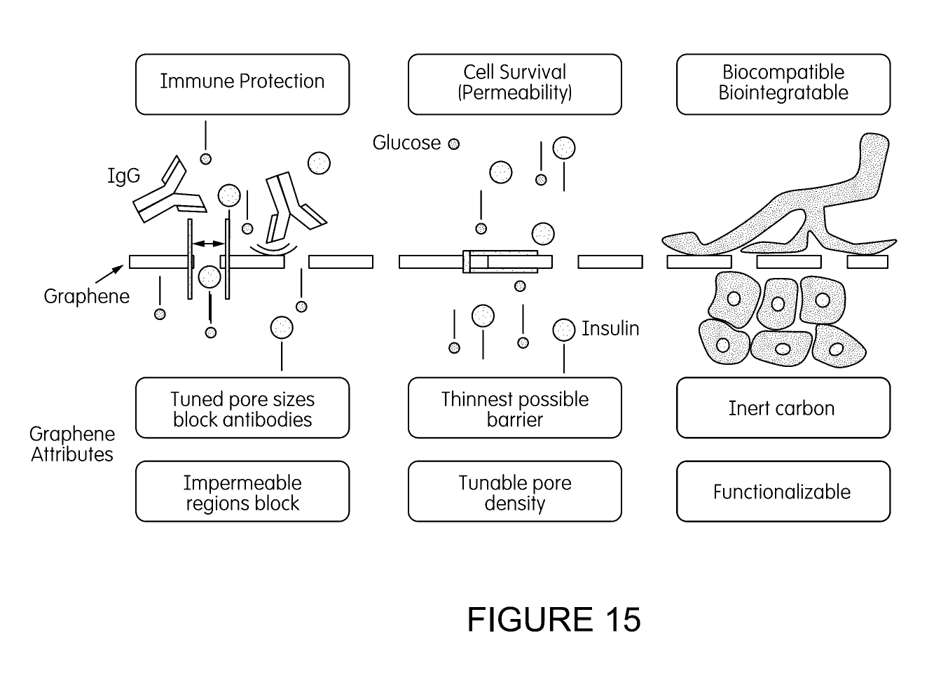

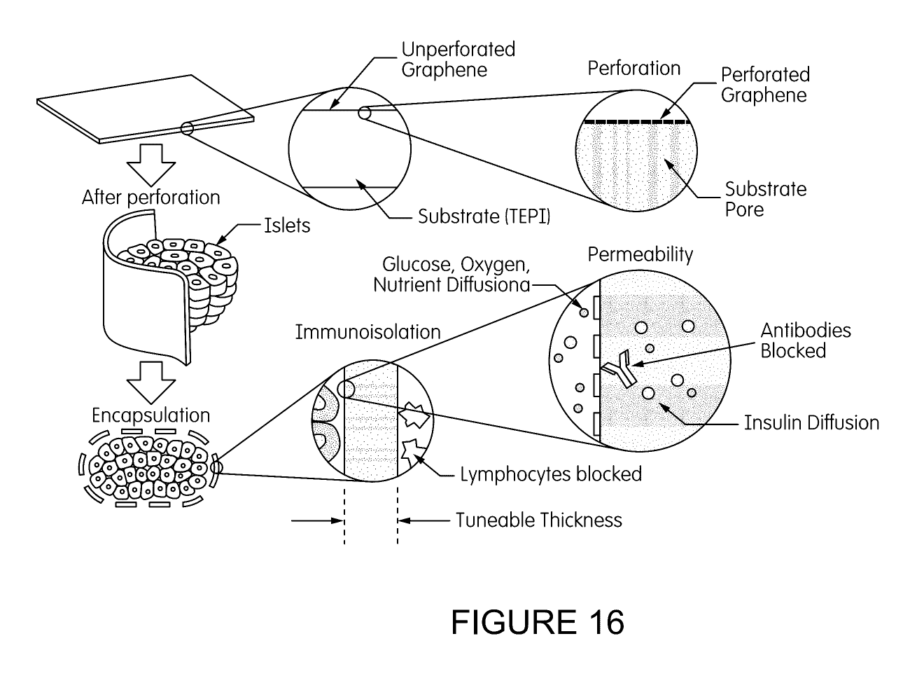

In some embodiments, the enclosure is not permeable to cells (such as immune cells), viruses, bacteria, antibodies, and/or complements of the immune system. Thus, in some embodiments, cells from the external environment cannot enter the enclosure and cells in the enclosure are retained. In some embodiments, the enclosure is permeable to desirable products, such as growth factors or hormones produced by the cells (see, e.g., FIGS. 15 and 16, illustrating some embodiments related to immunoisolation). The cells within the enclosure can be immunoisolated (i.e., protected from an immune reaction). In some embodiments of enclosures containing cells, the cells are yeast cells, bacterial cells, stem cells, mammalian cells, human cells, porcine cells, or a combination thereof. In some embodiments useful with cells, an enclosure comprises a plurality of sub-compartments, with the cells being positioned within one or more sub-compartments. In some embodiments useful with cells, the enclosure comprises a single compartment. In some embodiments, hole sizes in perforated two-dimensional materials useful for immunoisolation range in size from 1-50 nm, 1-40 nm, 1-30 nm, 1-25 nm, 1-17 nm, 1-15 nm, 1-12 nm, 1-10 nm, 3-50 nm, 3-30 nm, 3-20 nm, 3-10 nm, or 3-5 nm. In some embodiments, the size of the holes is about 1 nm, about 3 nm about 5 nm, about 10 nm, about 15 nm, about 20 nm, about 30 nm, or about 50 nm.

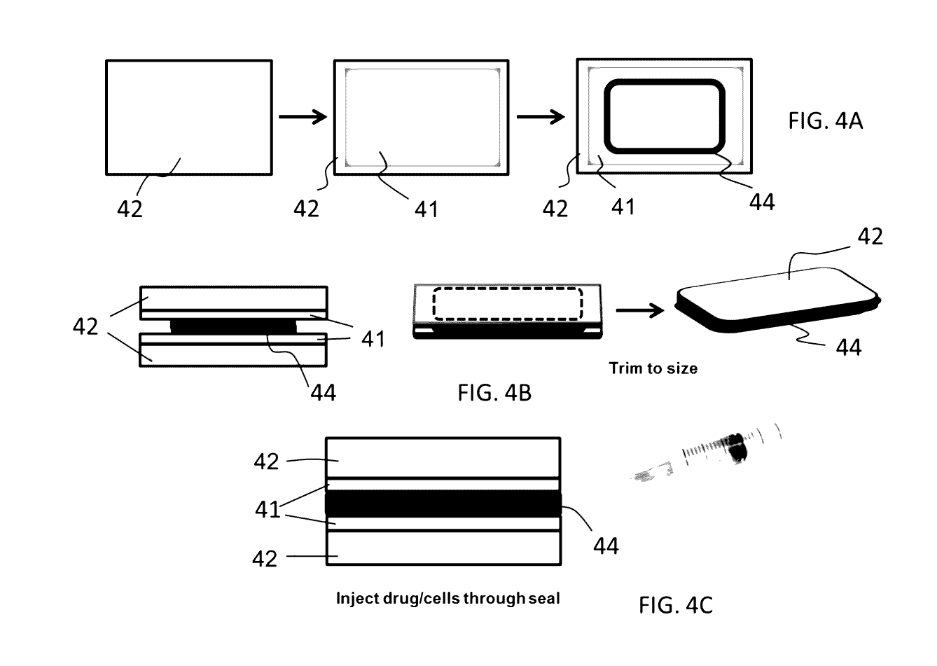

FIGS. 4A-4C illustrate an exemplary method for forming an enclosure and introducing selected substances, for example cells, therein. The method is illustrated with use of a sealant for forming the enclosure. As illustrated in FIG. 4A, a first composite layer or sheet can be formed by placing a sheet or layer of two-dimensional material, such as a sheet of graphene-based material or a sheet of graphene (41), in contact with a substrate layer (42). At least a portion of the substrate layer (42) of the first composite can be porous or permeable. Pore size of the substrate layer can be larger than the holes or apertures in the two-dimensional material employed and can be tuned for the environment (e.g., body cavity). A layer of sealant (44), e.g., silicone, is applied on the sheet or layer of perforated two-dimensional material outlining a compartment of the enclosure wherein the sealant will form a non-permeable seal around a perimeter of the enclosure. Formation of a single compartment is illustrated in FIGS. 4A-4C, however, it will be appreciated that multiple independent compartments within an enclosure can be formed by an analogous process. A second composite layer formed in the same way as the first is then prepared and positioned with the sheet or layer of perforated two dimensional materials in contact with the sealant. Alternatively, a sealant can be applied to a portion of a composite layer and the layer can be folded over in contact with the sealant to form an enclosure. A seal is then formed between the two composite layers. Appropriate pressure can be applied to facilitate sealing without damaging the two-dimensional material or its support. It will be appreciated that an alternative enclosure can be formed by applying a sheet or layer of non-porous and non-permeable support material in contact with the sealant. In this case only a portion of the enclosure is porous and permeable. Other methods for sealing the enclosure include ultrasonic welding. Sealed composite layers are illustrated in FIG. 4B where it is shown that the sealed layers can be trimmed to size around the sealant to form the enclosure. The enclosure formed is shown to have an external porous substrate layer 42 with the sheet or layer of perforated two-dimensional material (41) being positioned as an internal layer, with sealant 44 around the perimeter of the enclosure. As illustrated in FIG. 4C, cells or other substances that would be excluded from passage through the perforated two-dimensional sheet or layer can be introduced into the enclosure after it is formed by injection through the sealant layer. Any perforation formed by such injection can be sealed as needed.

In some embodiments, substances (e.g., cells) can be introduced into the enclosure prior to formation of the seal. In some embodiments, one or more ports can be provided for introducing substances into the enclosure. For example, a loading port can be provided within the sealed perimeter of the enclosure, and the loading port can be permanently or semi-permanently sealed after introduction of one or more substances through the loading port. Those in the art will appreciate that sterilization methods appropriate for the application envisioned can be employed during or after the preparation of the enclosure.

In some embodiments, an enclosure comprises perforated two-dimensional material encapsulating a substance, such that the substance is released to an environment external to the enclosure by passage through the holes in the perforated two-dimensional material. In some embodiments, the enclosure encapsulates two or more different substances. In some embodiments, not all of the different substances are released to an environment external to the enclosure. In some embodiments, all of the different substances are released into an environment external to the enclosure. In some embodiments, different substances are released into an environment external to the enclosure at different rates. In some embodiments, different substances are released into an environment external to the enclosure at the same rates.

In some embodiments of any enclosure herein at least a portion of the holes in the two-dimensional material of the enclosure are functionalized.

In some embodiments at least a portion of the two-dimensional material is conductive and a voltage can be applied to at least a portion of the conductive two-dimensional material. The voltage can be an AC or DC voltage. The voltage can be applied from a source external to the enclosure. In some embodiments, an enclosure device further comprises connectors and leads for application of a voltage from an external source to the two-dimensional material.

Additionally, the conductive properties of graphene-based or other two-dimensional materials can allow for electrification to take place from an external source. In exemplary embodiments, an AC or DC voltage can be applied to conductive two-dimensional materials (e.g., in a device such as an enclosure device). The conductivity properties of graphene can provide additional gating to charged molecules or substances. Electrification can occur permanently or only a portion of the time to affect gating. Directional gating of charged molecules can be directed not only through the pores (or restrict travel through pores), but also to the surface of the graphene to adsorb or bind and encourage growth, promote formation of a protective layer, or provide the basis or mechanism for other biochemical effects (e.g., on the body).

In some embodiments, at least once wall, or portion thereof, of the enclosure allows for electrostatic control of charged species, for instance in nanofluidic or microfluidic systems. In some embodiments, the wall allows for control of charged species by varying the applied voltage, for instance in nanofluidic or microfluidic systems. In some embodiments, the wall can be tuned to manipulate ion passage at low and/or high ion concentrations. In some embodiments, the wall is an ion-selective membrane. In some embodiments, the wall comprises one or more voltage-gated ion channels, such as voltage-gated pores. In some embodiments, the wall mimics biological voltage-gated ion channels. In some embodiments, the wall is a solid-state membrane. In some embodiments, nanochannel or nanopore transistors can be used to manipulate ion passage.

In some embodiments, the wall can be tuned using low or high applied voltages. In some embodiments, the wall allows high ionic flux. In some embodiments, the wall allows low ion flux. In some embodiments, pores in the wall modulates current of ions at low gate voltages and/or display high selectivity. In some embodiments, ion flux across the wall can be turned on or off at low applied voltages, such as ≦500 mV. In some embodiments, ion flux across the wall can be turned on or off at biologically relevant ion concentrations, such as up to 1 M. In some embodiments, the applied voltage can modulate on species selectivity, e.g., cation or anion selectivity.

In some embodiments, nanopores can be electrostatically controlled at low voltages and biologically relevant ion concentrations. In some embodiments, walls are used in separation and sensing technologies. In some embodiments, walls are used in water filtration, water desalination, water purification, osmosis, energy storage, microfluidic devices, nanofluidic devices, and/or therapeutic methods. In some embodiments, walls are used in immune-isolation (i.e., protecting substances from an immune reaction), timed drug release (e.g., sustained or delayed release), hemodialysis, and hemofiltration. Some embodiments relate to methods for separating ions or other substances; methods for sensing ions; methods for storing energy; methods for filtering water; and/or methods of treating a disease or condition (e.g., diabetes). Some embodiments relate to methods of ultrafiltration, nanofiltration and/or microfiltration. Some embodiments comprise using gating to control release of substances. Some embodiments comprise using gating to allow for different substances to be released at different times. Some embodiments comprise allowing different substances to pass through the wall at different times, thus modulating when and how substances mix and interact with other substances in a specific order.

Some embodiments comprise a method comprising introducing an enclosure comprising perforated two-dimensional material into an environment, the enclosure containing at least one substance; and releasing at least a portion of the at least one substance through the holes of the two-dimensional material to the environment external to the enclosure. In some embodiments, the enclosure contains cells which are not released from the enclosure and the at least one substance, a portion of which is released, is a substance generated by the cells in the enclosure.

Some embodiments comprise a method comprising introducing an enclosure comprising perforated two-dimensional material to an environment, the enclosure containing at least one first substance; and allowing migration of other substances from the environment into the enclosure. In some embodiments, the first substance is cells, and other substances include nutrients and/or oxygen.

In some embodiments, a composite structure comprises perforated two-dimensional material and a first fibrous layer comprising a plurality of polymer filaments affixed to a surface of the two-dimensional material; wherein the composite structure is substantially planar. In some embodiments, the perforated two-dimensional material has a second fibrous layer affixed to a surface of the two-dimensional material opposite the first fibrous layer. In some embodiments, the average pore size of the first fibrous layer is different from the average pore size of the second fibrous layer. In some embodiments, the first and/or second fibrous layer comprises an additive selected from the group consisting of pharmaceuticals, cells, growth factors, clotting factors, blood thinners, immunosuppressants, antimicrobial agents, hormones, antibodies, minerals, nutrients and combinations thereof. In some embodiments, the substantially planar composite structure is flexible. In some embodiments, the substantially planar composite structure is rigid. In some embodiments, multiple composite structures are combined to form a pouch-like enclosure. Such planar composite structures can be useful, for example, as appliques for wound healing. The composite structures can also be used, for example, as a component of an adhesive bandage.

Both permanent and temporary binding of substances to the enclosure are possible. In some embodiments, enclosures represent a disruptive technology for state of the art vehicle and other devices, such that these vehicles and devices to be used in new ways. For example, cell line developments, therapeutic releasing agents, and sensing paradigms (e.g., MRSw's, NMR-based magnetic relaxation switches, see; Koh et al. (2008) Ang. Chem. Int'l Ed. Engl., 47(22) 4119-4121) can be used. Moreover, some embodiments mitigate biofouling and bioreactivity, convey superior permeability and less delay in response, and provide mechanical stability.

In some embodiments, enclosures can be used in non-therapeutic applications, such as in dosing probiotics in dairy products.

In some embodiments, two-dimensional materials are atomically thin, with thickness ranging from single-layer sub-nanometer thickness to a few nanometers. Two-dimensional materials include metal chalogenides (e.g., transition metal dichalogenides), transition metal oxides, hexagonal boron nitride, graphene, silicene and germanene (see: Xu et al. (2013) “Graphene-like Two-Dimensional Materials) Chemical Reviews 113:3766-3798).

In some embodiments, the two-dimensional material comprises a graphene-based material.

Graphene represents a form of carbon in which the carbon atoms reside within a single atomically thin sheet or a few layered sheets (e.g., about 20 or less) of fused six-membered rings forming an extended sp²-hybridized carbon planar lattice. Graphene-based materials include, but are not limited to, single layer graphene, multilayer graphene or interconnected single or multilayer graphene domains and combinations thereof. In some embodiments, graphene-based materials also include materials which have been formed by stacking single or multilayer graphene sheets. In some embodiments, multilayer graphene includes 2 to 20 layers, 2 to 10 layers or 2 to 5 layers. In some embodiments, layers of multilayered graphene are stacked, but are less ordered in the z direction (perpendicular to the basal plane) than a thin graphite crystal.

In some embodiments, a sheet of graphene-based material may be a sheet of single or multilayer graphene or a sheet comprising a plurality of interconnected single or multilayer graphene domains, which may be observed in any known manner such as using for example small angle electron diffraction, transmission electron microscopy, etc. In some embodiments, the multilayer graphene domains have 2 to 5 layers or 2 to 10 layers. As used herein, a domain refers to a region of a material where atoms are substantially uniformly ordered into a crystal lattice. A domain is uniform within its boundaries, but may be different from a neighboring region. For example, a single crystalline material has a single domain of ordered atoms. In some embodiments, at least some of the graphene domains are nanocrystals, having domain size from 1 to 100 nm or 10-100 nm. In some embodiments, at least some of the graphene domains have a domain size greater than from 100 nm to 1 cm, or from 100 nm to 1 micron, or from 200 nm to 800 nm, or from 300 nm to 500 nm. In some embodiments, a domain of multilayer graphene may overlap a neighboring domain. Grain boundaries formed by crystallographic defects at edges of each domain may differentiate between neighboring crystal lattices. In some embodiments, a first crystal lattice may be rotated relative to a second crystal lattice, by rotation about an axis perpendicular to the plane of a sheet, such that the two lattices differ in crystal lattice orientation.

In some embodiments, the sheet of graphene-based material is a sheet of single or multilayer graphene or a combination thereof. In some other embodiments, the sheet of graphene-based material is a sheet comprising a plurality of interconnected single or multilayer graphene domains. In some embodiments, the interconnected domains are covalently bonded together to form the sheet. When the domains in a sheet differ in crystal lattice orientation, the sheet is polycrystalline.

In some embodiments, the thickness of the sheet of graphene-based material is from 0.3 to 10 nm, 0.34 to 10 nm, from 0.34 to 5 nm, or from 0.34 to 3 nm. In some embodiments, the thickness includes both single layer graphene and the non-graphenic carbon.

In some embodiments, a sheet of graphene-based material comprises intrinsic or native defects. Intrinsic or native defects may result from preparation of the graphene-based material in contrast to perforations which are selectively introduced into a sheet of graphene-based material or a sheet of graphene. Such intrinsic or native defects may include, but are not limited to, lattice anomalies, pores, tears, cracks or wrinkles. Lattice anomalies can include, but are not limited to, carbon rings with other than 6 members (e.g. 5, 7 or 9 membered rings), vacancies, interstitial defects (including incorporation of non-carbon atoms in the lattice), and grain boundaries. Perforations are distinct from openings in the graphene lattice due to intrinsic or native defects or grain boundaries, but testing and characterization of the final membrane such as mean pore size and the like encompasses all openings regardless of origin since they are all present.

In some embodiments, graphene is the dominant material in a graphene-based material. For example, a graphene-based material may comprise at least 20% graphene, at least 30% graphene, or at least 40% graphene, or at least 50% graphene, or at least 60% graphene, or at least 70% graphene, or at least 80% graphene, or at least 90% graphene, or at least 95% graphene. In some embodiments, a graphene-based material comprises a range of graphene selected from 30% to 95%, or from 40% to 80% from 50% to 70%, from 60% to 95% or from 75% to 100%. The amount of graphene in the graphene-based material is quantified as an atomic percentage utilizing known methods including scanning transmission electron microscope examination, or alternatively if STEM or TEM is ineffective another similar measurement technique.

In some embodiments, a sheet of graphene-based material further comprises non-graphenic carbon-based material located on at least one surface of the sheet of graphene-based material. In some embodiments, the sheet is exemplified by two base surfaces (e.g. top and bottom faces of the sheet, opposing faces) and side faces (e.g. the side faces of the sheet). In some further embodiments, the “bottom” face of the sheet is that face which contacted the substrate during growth of the sheet and the “free” face of the sheet opposite the “bottom” face. In some embodiments, non-graphenic carbon-based material may be located on one or both base surfaces of the sheet (e.g. the substrate side of the sheet and/or the free surface of the sheet). In some further embodiments, the sheet of graphene-based material includes a small amount of one or more other materials on the surface, such as, but not limited to, one or more dust particles or similar contaminants.

In some embodiments, the amount of non-graphenic carbon-based material is less than the amount of graphene. In some further embodiments, the amount of non-graphenic carbon material is three to five times the amount of graphene; this is measured in terms of mass. In some additional embodiments, the non-graphenic carbon material is characterized by a percentage by mass of said graphene-based material selected from the range of 0% to 80%. In some embodiments, the surface coverage of the sheet of non-graphenic carbon-based material is greater than zero and less than 80%, from 5% to 80%, from 10% to 80%, from 5% to 50% or from 10% to 50%. This surface coverage may be measured with transmission electron microscopy, which gives a projection. In some embodiments, the amount of graphene in the graphene-based material is from 60% to 95% or from 75% to 100%. The amount of graphene in the graphene-based material is measured as an atomic percentage utilizing known methods preferentially using transmission electron microscope examination, or alternatively if STEM is ineffective using an atomic force microscope.

In some embodiments, the non-graphenic carbon-based material does not possess long range order and is classified as amorphous. In some embodiments, the non-graphenic carbon-based material further comprises elements other than carbon and/or hydrocarbons. In some embodiments, non-carbon elements which may be incorporated in the non-graphenic carbon include hydrogen, oxygen, silicon, copper, and iron. In some further embodiments, the non-graphenic carbon-based material comprises hydrocarbons. In some embodiments, carbon is the dominant material in non-graphenic carbon-based material. For example, a non-graphenic carbon-based material in some embodiments comprises at least 30% carbon, or at least 40% carbon, or at least 50% carbon, or at least 60% carbon, or at least 70% carbon, or at least 80% carbon, or at least 90% carbon, or at least 95% carbon. In some embodiments, a non-graphenic carbon-based material comprises a range of carbon selected from 30% to 95%, or from 40% to 80%, or from 50% to 70%. The amount of carbon in the non-graphenic carbon-based material is measured as an atomic percentage utilizing known methods preferentially using transmission electron microscope examination, or alternatively if STEM is ineffective using atomic force microscope.

Perforation techniques suitable for use in perforating the graphene-based materials may include described herein ion-based perforation methods and UV-oxygen based methods.

Ion-based perforation methods include methods in which the graphene-based material is irradiated with a directional source of ions. In some further embodiments, the ion source is collimated. In some embodiments, the ion source is a broad beam or flood source. A broad field or flood ion source can provide an ion flux which is significantly reduced compared to a focused ion beam. The ion source inducing perforation of the graphene or other two-dimensional material is considered to provide a broad ion field, also commonly referred to as an ion flood source. In some embodiments, the ion flood source does not include focusing lenses. In some embodiments, the ion source is operated at less than atmospheric pressure, such as at 10⁻³ to 10⁻⁵ torr or 10⁻⁴ to 10⁻⁶ torr. In some embodiments, the environment also contains background amounts (e.g. on the order of 10⁻⁵ torr) of oxygen (O₂), nitrogen (N₂) or carbon dioxide (CO₂). In some embodiments, the ion beam may be perpendicular to the surface of the layer(s) of the material (incidence angle of 0 degrees) or the incidence angle may be from 0 to 45 degrees, 0 to 20 degrees, 0 to 15 degrees or 0 to 10 degrees. In some further embodiments, exposure to ions does not include exposure to plasma.

In some embodiments, UV-oxygen based perforation methods include methods in which the graphene-based material is simultaneously exposed to ultraviolet (UV) light and an oxygen containing gas Ozone may be generated by exposure of an oxygen containing gas such as oxygen or air to the UV light. Ozone may also be supplied by an ozone generator device. In some embodiments, the UV-oxygen based perforation method further includes exposure of the graphene-based material to atomic oxygen. Suitable wavelengths of UV light include, but are not limited to wavelengths below 300 nm or from 150 nm to 300 nm. In some embodiments, the intensity from 10 to 100 mW/cm² at 6 mm distance or 100 to 1000 mW/cm² at 6 mm distance. For example, suitable light is emitted by mercury discharge lamps (e.g. about 185 nm and 254 nm). In some embodiments, UV/oxygen cleaning is performed at room temperature or at a temperature greater than room temperature. In some further embodiments, UV/oxygen cleaning is performed at atmospheric pressure (e.g. 1 atm) or under vacuum.

Perforations are sized as described herein to provide desired selective permeability of a species (atom, molecule, protein, virus, cell, etc.) for a given application. Selective permeability relates to the propensity of a porous material or a perforated two-dimensional material to allow passage (or transport) of one or more species more readily or faster than other species. Selective permeability allows separation of species which exhibit different passage or transport rates. In two-dimensional materials selective permeability correlates to the dimension or size (e.g., diameter) of apertures and the relative effective size of the species. Selective permeability of the perforations in two-dimensional materials such as graphene-based materials can also depend on functionalization of perforations (if any) and the specific species. Separation or passage of two or more species in a mixture includes a change in the ratio(s) (weight or molar ratio) of the two or more species in the mixture during and after passage of the mixture through a perforated two-dimensional material.

In some embodiments, the characteristic size of the perforation is from 0.3 to 10 nm, from 1 to 10 nm, from 5 to 10 nm, from 5 to 20 nm, from 10 nm to 50 nm, from 50 nm to 100 nm, from 50 nm to 150 nm, from 100 nm to 200 nm, or from 100 nm to 500 nm. In some embodiments, the average pore size is within the specified range. In some embodiments, 70% to 99%, 80% to 99%, 85% to 99% or 90 to 99% of the perforations in a sheet or layer fall within a specified range, but other pores fall outside the specified range.

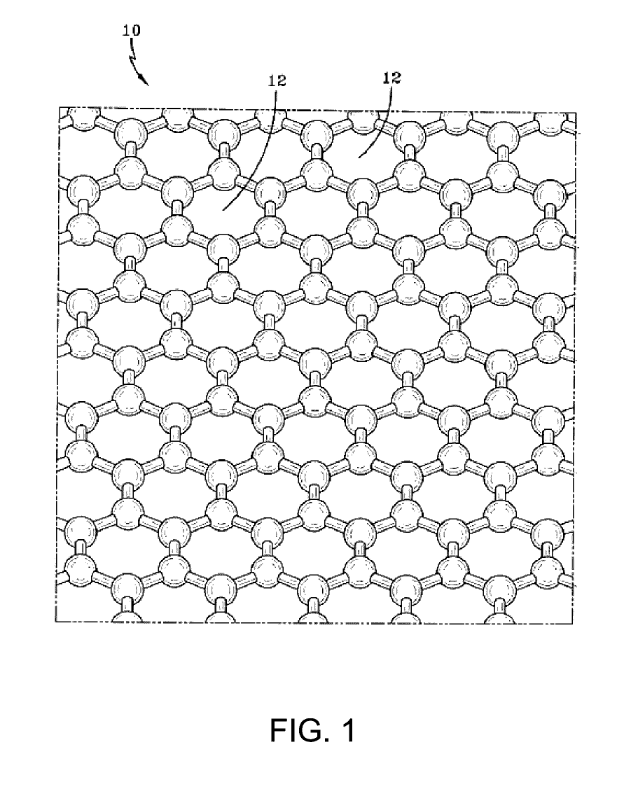

Nanomaterials in which pores are intentionally created may be referred to as perforated graphene, perforated graphene-based materials or perforated two-dimensional materials, and the like. Perforated graphene-based materials include materials in which non-carbon atoms have been incorporated at the edges of the pores. Pore features and other material features may be characterized in a variety of manners including in relation to size, area, domains, periodicity, coefficient of variation, etc. For instance, the size of a pore may be assessed through quantitative image analysis utilizing images preferentially obtained through transmission electron microscopy, and if TEM is ineffective, through atomic force microscopy, and if AFM is ineffective, through scanning electron microscopy, as for example presented in FIGS. 1 and 2. The boundary of the presence and absence of material identifies the contour of a pore. The size of a pore may be determined by shape fitting of an expected species against the imaged pore contour where the size measurement is characterized by smallest dimension unless otherwise specified. For example, in some instances, the shape may be round or oval. The round shape exhibits a constant and smallest dimension equal to its diameter. The width of an oval is its smallest dimension. The diameter and width measurements of the shape fitting in these instances provide the size measurement, unless specified otherwise.

Each pore size of a test sample may be measured to determine a distribution of pore sizes within the test sample. Other parameters may also be measured such as area, domain, periodicity, coefficient of variation, etc. Multiple test samples may be taken of a larger membrane to determine that the consistency of the results properly characterizes the whole membrane. In such instance, the results may be confirmed by testing the performance of the membrane with test species. For example, if measurements indicate that certain sizes of species should be restrained from transport across the membrane, a performance test provides verification with test species. Alternatively, the performance test may be utilized as an indicator that the pore measurements will determine a concordant pore size, area, domains, periodicity, coefficient of variation, etc.

The size distribution of holes may be narrow, e.g., limited to 0.1-0.5 coefficient of variation. In some embodiments, the characteristic dimension of the holes is selected for the application.

In some embodiments involving circular shape fitting the equivalent diameter of each pore is calculated from the equation A=πd²/4. Otherwise, the area is a function of the shape fitting. When the pore area is plotted as a function of equivalent pore diameter, a pore size distribution may be obtained. The coefficient of variation of the pore size may be calculated herein as the ratio of the standard deviation of the pore size to the mean of the pore size as measured across the test samples. The average area of perforations is an averaged measured area of pores as measured across the test samples.

In some embodiments, the ratio of the area of the perforations to the ratio of the area of the sheet may be used to characterize the sheet as a density of perforations. The area of a test sample may be taken as the planar area spanned by the test sample. Additional sheet surface area may be excluded due to wrinkles other non-planar features. Characterization may be based on the ratio of the area of the perforations to the test sample area as density of perforations excluding features such as surface debris. Characterization may be based on the ratio of the area of the perforations to the suspended area of the sheet. As with other testing, multiple test samples may be taken to confirm consistency across tests and verification may be obtained by performance testing. The density of perforations may be, for example, 2 per nm² (2/nm²) to 1 per μm²(1/μm²).

In some embodiments, the perforated area comprises 0.1% or greater, 1% or greater or 5% or greater of the sheet area, less than 10% of the sheet area, less than 15% of the sheet area, from 0.1% to 15% of the sheet area, from 1% to 15% of the sheet area, from 5% to 15% of the sheet area or from 1% to 10% of the sheet area. In some further embodiments, the perforations are located over greater than 10% or greater than 15% of said area of said sheet of graphene-based material. A macroscale sheet is macroscopic and observable by the naked eye. In some embodiments, at least one lateral dimension of the sheet is greater than 3 cm, greater than 1 cm, greater than 1 mm or greater than 5 mm. In some further embodiments, the sheet is larger than a graphene flake which would be obtained by exfoliation of graphite in known processes used to make graphene flakes. For example, the sheet has a lateral dimension greater than about 1 micrometer. In an additional embodiment, the lateral dimension of the sheet is less than 10 cm. In some further embodiments, the sheet has a lateral dimension (e.g., perpendicular to the thickness of the sheet) from 10 nm to 10 cm or greater than 1 mm and less than 10 cm.

Chemical vapor deposition growth of graphene-based material typically involves use of a carbon containing precursor material, such as methane and a growth substrate. In some embodiments, the growth substrate is a metal growth substrate. In some embodiments, the metal growth substrate is a substantially continuous layer of metal rather than a grid or mesh. Metal growth substrates compatible with growth of graphene and graphene-based materials include transition metals and their alloys. In some embodiments, the metal growth substrate is copper based or nickel based. In some embodiments, the metal growth substrate is copper or nickel. In some embodiments, the graphene-based material is removed from the growth substrate by dissolution of the growth substrate.

In some embodiments, the sheet of graphene-based material is formed by chemical vapor deposition (CVD) followed by at least one additional conditioning or treatment step. In some embodiments, the conditioning step is selected from thermal treatment, UV-oxygen treatment, ion beam treatment, and combinations thereof. In some embodiments, thermal treatment may include heating to a temperature from 200° C. to 800° C. at a pressure of 10⁻⁷ torr to atmospheric pressure for a time of 2 hours to 8 hours. In some embodiments, UV-oxygen treatment may involve exposure to light from 150 nm to 300 nm and an intensity from 10 to 100 mW/cm² at 6 mm distance for a time from 60 to 1200 seconds. In some embodiments, UV-oxygen treatment may be performed at room temperature or at a temperature greater than room temperature. In some further embodiments, UV-oxygen treatment may be performed at atmospheric pressure (e.g. 1 atm) or under vacuum. In some embodiments, ion beam treatment may involve exposure of the graphene-based material to ions having an ion energy from 50 eV to 1000 eV (for pretreatment) and the fluence is from 3×10¹⁰ ions/cm² to 8×10¹¹ ions/cm² or 3×10¹⁰ ions/cm² to 8×10¹³ ions/cm² (for pretreatment). In some further embodiments, the source of ions may be collimated, such as a broad beam or flood source. In some embodiments, the ions may be noble gas ions such as Xe⁺. In some embodiments, one or more conditioning steps are performed while the graphene-based material is attached to a substrate, such as a growth substrate.

In some embodiments, the conditioning treatment affects the mobility and/or volatility of the non-graphitic carbon-based material. In some embodiments, the surface mobility of the non-graphenic carbon-based material is such that when irradiated with perforation parameters such as described herein, the non-graphenic carbon-based material, may have a surface mobility such that the perforation process results ultimately in perforation. Without wishing to be bound by any particular belief, hole formation is believed to be related to beam induced carbon removal from the graphene sheet and thermal replenishment of carbon in the hole region by non graphenic carbon. The replenishment process may be dependent upon energy entering the system during perforation and the resulting surface mobility of the non-graphenic carbon based material. To form holes, the rate of graphene removal may be higher than the non-graphenic carbon hole filling rate. These competing rates depend on the non-graphenic carbon flux (e.g., mobility [viscosity and temperature] and quantity) and the graphene removal rate (e.g., particle mass, energy, flux).

In some embodiments, the volatility of the non-graphenic carbon-based material may be less than that which is obtained by heating the sheet of graphene-based material to 500° C. for 4 hours in vacuum or at atmospheric pressure with an inert gas.

In various embodiments, CVD graphene or graphene-based material can be liberated from its growth substrate (e.g., Cu) and transferred to a supporting grid, mesh or other supporting structure. In some embodiments, the supporting structure may be configured so that at least some portions of the sheet of graphene-based material are suspended from the supporting structure. For example, at least some portions of the sheet of graphene-based material may not be in contact with the supporting structure.

In some embodiments, the sheet of graphene-based material following chemical vapor deposition comprises a single layer of graphene having at least two surfaces and non-graphenic carbon based material may be provided on said surfaces of the single layer graphene. In some embodiments, the non-graphenic carbon based material may be located on one of the two surfaces or on both. In some further embodiments, additional graphenic carbon may also present on the surface(s) of the single layer graphene.

In embodiments of the disclosure herein, the particle beam is a nanoparticle beam or cluster beam. In further embodiments, the beam is collimated or is not collimated.

Furthermore, the beam need not be highly focused. In some embodiments, a plurality of the nanoparticles or clusters is singly charged. In additional embodiments, the nanoparticles comprise from 500 to 250,000 atoms or from 500 to 5,000 atoms.

A variety of metal particles are suitable for use in the methods of the present disclosure. For example, nanoparticles of Al, Ag, Au, Ti, Cu and nanoparticles comprising Al, Ag, Au, Ti, Cu are suitable. Metal NPs can be generated in a number of ways including magnetron sputtering and liquid metal ion sources (LMIS). Methods for generation of nanoparticles are further described in Cassidy, Cathal, et al. “Inoculation of silicon nanoparticles with silver atoms.” Scientific reports 3 (2013), Haberland, Hellmut, et al. “Filling of micron-sized contact holes with copper by energetic cluster impact.” Journal of Vacuum Science & Technology A 12.5 (1994): 2925-2930, Bromann, Karsten, et al. “Controlled deposition of size-selected silver nanoclusters.” Science 274.5289 (1996): 956-958, Palmer, R. E., S. Pratontep, and H-G. Boyen. “Nanostructured surfaces from size-selected clusters.” Nature Materials 2.7 (2003): 443-448, Shyjumon, I., et al. “Structural deformation, melting point and lattice parameter studies of size selected silver clusters.” The European Physical Journal D-Atomic, Molecular, Optical and Plasma Physics 37.3 (2006): 409-415, Allen, L. P., et al. “Craters on silicon surfaces created by gas cluster ion impacts.” Journal of applied physics 92.7 (2002): 3671-3678, Wucher, Andreas, Hua Tian, and Nicholas Winograd. “A Mixed Cluster Ion Beam to Enhance the Ionization Efficiency in Molecular Secondary Ion Mass Spectrometry.” Rapid communications in mass spectrometry: RCM 28.4 (2014): 396-400. PMC. Web. 6 Aug. 2015 and Pratontep, S., et al. “Size-selected cluster beam source based on radio frequency magnetron plasma sputtering and gas condensation.” Review of scientific instruments 76.4 (2005): 045103, each of which is hereby incorporated by reference for its description of nanoparticle generation techniques.

Gas cluster beams can be made when high pressure gas adiabatically expands in a vacuum and cools such that it condenses into clusters. Clusters can also be made ex situ such as C60 and then accelerated towards the graphene.

In some embodiments, the nanoparticles are specially selected to introduce moieties into the graphene. In some embodiments, the nanoparticles function as catalysts. The moieties may be introduced at elevated temperatures, optionally in the presence of a gas. In other embodiments, the nanoparticles introduce“chiseling” moieties, which are moieties that help reduce the amount of energy needed to remove an atom during irradiation.

In embodiments, the size of the perforation apertures is controlled by controlling both the nanoparticle size and the nanoparticle energy. Without wishing to be bound by any particular belief, if all the nanoparticles have sufficient energy to perforate, then the resulting perforation is believed to correlated with the nanoparticle sizes selected. However, the size of the perforation is believed to be influenced by deformation of the nanoparticle during the perforation process. This deformation is believed to be influenced by both the energy and size of the nanoparticle and the stiffness of the graphene layer(s). A grazing angle of incidence of the nanoparticles can also produce deformation of the nanoparticles. In addition, if the nanoparticle energy is controlled, it is believed that nanoparticles can be deposited with a large mass and size distribution, but that a sharp cutoff can still be achieved.

Without wishing to be bound by any particular belief, the mechanism of perforation is believed to be a continuum bound by sputtering on one end (where enough energy is delivered to the graphene sheet to atomize the carbon in and around the NP impact site) and ripping or fracturing (where strain induced failure opens a torn hole but leaves the graphene carbons as part of the original sheet). Part of the graphene layer may fold over at the site of the rip or fracture. In an embodiment the cluster can be reactive so as to aid in the removal of carbon (e.g. an oxygen cluster or having trace amounts of a molecule known to etch carbon in a gas cluster beam i.e. a mixed gas cluster beam). Without wishing to be bound by any particular belief, the stiffness of a graphene layer is believed to be influenced by both the elastic modulus of graphene and the tautness of the graphene. Factors influencing the elastic modulus of a graphene layer are believed to include temperature, defects (either small defects or larger defects from NP irradiation), physisorption, chemisorption and doping. Tautness is believed to be influenced by coefficient of thermal expansion mismatches (e.g. between substrate and graphene layer) during deposition, strain in the graphene layer, wrinkling of the graphene layer. It is believed that strain in a graphene layer can be influenced by a number of factors including application of gas pressure to the backside (substrate side) of a graphene layer, straining of an elastic substrate prior to deposition of graphene, flexing of the substrate during deposition, and defecting the graphene layer in controlled regions to cause the layer to locally contract and increase the local strain.

In embodiments, nanoparticle perforation can be further controlled by straining the graphene layers during perforation to induce fracture, thereby “ripping” or “tearing” one or more graphene layers. In some embodiments, the stress is directional and used to preferentially fracture in a specific orientation. For example, ripping of one or more graphene sheets can be used to create “slit” shaped apertures; such apertures can be substantially larger than the nanoparticle used to initiate the aperture. In additional embodiments, the stress is not oriented in a particular direction.

In some embodiments, enclosures can be further modified to incorporate any number of variations, alterations, substitutions or equivalent arrangements not heretofore described, but which are commensurate with the spirit and scope of the disclosure. Accordingly, the enclosures and methods are not limited by the foregoing description.

Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomer and enantiomer of the compound described individually or in any combination. One of ordinary skill in the art will appreciate that methods, device elements, starting materials and synthetic methods other than those specifically exemplified can be employed in the practice of the invention without resort to undue experimentation. All art-known functional equivalents, of any such methods, device elements, starting materials and synthetic methods are intended to be included in this invention. Whenever a range is given in the specification, for example, a temperature range, a time range, or a composition range, all intermediate ranges and subranges, as well as all individual values included in the ranges given are intended to be included in the disclosure. When a Markush group or other grouping is used herein, all individual members of the group and all combinations and subcombinations possible of the group are intended to be individually included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably can be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although some embodiments have been specifically disclosed, modification and variation of the concepts herein disclosed may be resorted to by those skilled in the art, and that such modifications and variations are considered to be within the scope of this invention as defined by the appended claims.

In general the terms and phrases used herein have their art-recognized meaning, which can be found by reference to standard texts, journal references and contexts known to those skilled in the art. The preceding definitions are provided to clarify their specific use in the context of the invention.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claims.

### WORKING EXAMPLES

Some embodiments are further illustrated by the following examples. The examples are provided for illustrative purposes only, and are not to be construed as limiting the scope or content of embodiments any way.

### Example 1

### Cytotoxicity Testing: Neural Red Uptake (NRU) Cytotoxicity

The in vitro bioreactivity of L929 mouse fibroblast cell cultures were quantitatively determined in response to an extract of test material. The cells were grown to semi-confluency in 96-well tissue culture plates, so that they formed a half-confluent monolayer. An extract of the test material was prepared in Minimum Essential Medium (MEM), dipped in ethanol and allowed to dry, and then transferred onto the cell layer in the culture plate. Positive and negative controls were prepared in the same way. All extracts were dosed in 6 replicates except for the controls, which were dosed in 12 replicates. Control materials were tested at 100% concentration (neat), and the test materials were tested at 100% (neat), 50%, 25%, and 12.5% concentrations.

The plates were incubated for 24 to 26 hours at 37±1° C. in a humified atmosphere containing 5±1% CO₂. After examination of the plates, the culture medium was carefully removed and the cells were washed with Phosphate Buffer Saline (PBS). 100 μL of Neutral Red (NR) medium (50 μg/mL in MEM without Phenol Red, filtered through a 0.2 μm filter and used the same day) was added to each test well, and the plates were further incubated for 3±0.2 hours in an incubator at 37±1° C. The cells were then washed with PBS and 100 μL of ethanol/acetic acid solution was added to each test well to extract the NR. The optical density (OD) of each well was measured at 540 nm.

The number of living cells was correlated to the intensity determined by photometric measurements after desorption of the NR. That is, a decrease in the number of living cells was directly correlated with the amount of NR, as monitored by absorbance at 540 nm. A reduction in viability of the test material as compared to a blank (i.e., cells exposed to extraction medium) was calculated using the following equation:

Viability %=100× OD₅₄₀ₑ/OD_{540b}, where OD₅₄₀ₑ is the mean value of the measured optical density of the extracts of the test material, positive control, or negative control; and OD_{540b} is the mean value of the measured optical density of the blanks. The test system was considered suitable if the following conditions were met: (a) the viability % for the negative control was ≧70%, (b) the viability % for the positive control was <70%, and (c) the mean of each replicate of the untreated control (i.e., each column in the tabulated results for the untreated control) was within ±15% of the untreated control mean. If the viability of test material sample was <70%, it was considered to have cytotoxic potential.

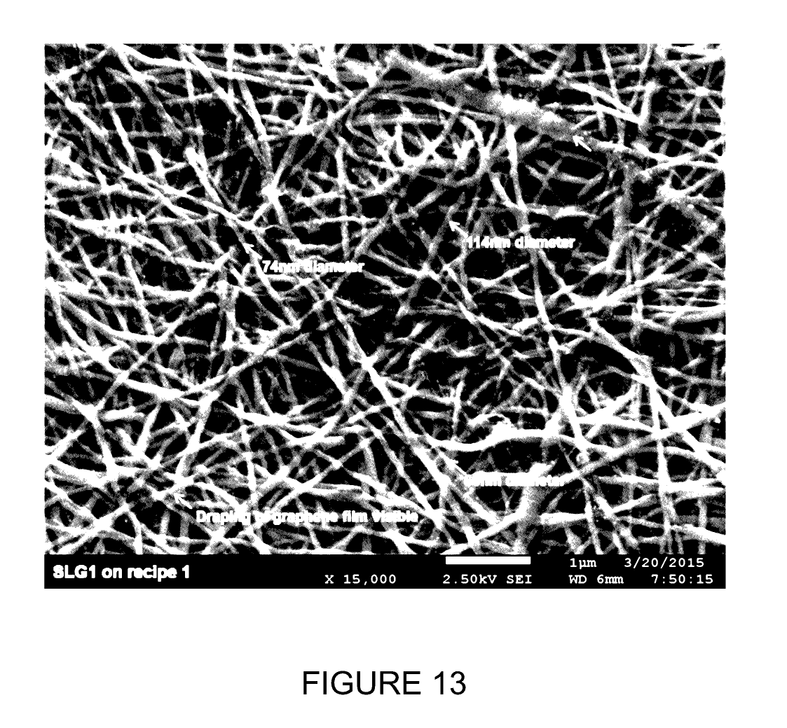

Cytotoxicity testing was conducted with the following test materials: (i) uncoated/bare substrate; (ii) unperforated graphene on substrate; and (iii) perforated graphene on substrate. Photographs of exemplary test materials are shown in FIG. 13. The substrate used in the test materials was track-etched polyimide. Perforated graphene was prepared via nanoparticle perforation. Briefly, Two layers of CVD graphene material were prepared. Each layer received ion beam treatment on Cu growth substrate, transfer to polymer (TEPI) for testing, UV-oxygen treatment and 300° C. bakeout for 8 hours before the layers were stacked. The stacked layers were exposed to 6.5 kV Ag nanoparticles (NP). The NP distribution was centered on 6 nm and the fluence was approximately 5×10¹⁰ NPs/cm². The nanoparticles were provided at an incidence angle of approximately greater than 45 degrees to the normal of the basal plane of the sheet of graphene material. The NPs were of 9-11 nm diameter at an energy of 30 keV. The pores were typically 10-12 nm at their base, and varied in length from about 20 nm to 70 mm.

### Uncoated/Bare Substrate

Cytotoxicity of the uncoated/bare substrate was tested using the parameters in Table 1.

TABLE 1
Parameters used to prepare test and control articles for
uncoated/bare substrate cytotoxicity experiments

| Sample | Amount | Vehicle | Volume | Ratio | Time/Temperature |
|---|---|---|---|---|---|
| Test Article | 22.9 cm² | complete MEM | 3.8 mL | 6 cm²/mL | 24 ± 2 hours at 37 ± 1° C. |
| Positive Control | 30 cm² | complete MEM | 10.0 mL | 3 cm²/mL | 24 ± 2 hours at 37 ± 1° C. |
| Negative Control | 30 cm² | complete MEM | 10.0 mL | 3 cm²/mL | 24 ± 2 hours at 37 ± 1° C. |
| Untreated Control | N/A | complete MEM | 10.0 mL | N/A | 24 ± 2 hours at 37 ± 1° C. |

N/A: Not Applicable

The results of the cytotoxicity testing are shown in Table 2.

TABLE 2
Cell viability results in experiments with uncoated/bare substrate

| | | | | | | Test Article | | | |
|---|---|---|---|---|---|---|---|---|---|
| | | Control Articles | | | | 100% | | | |
| Replicates | | Untreated | | Negative | Positive | (neat) | 50% | 25% | 12.5% |
| 1 | 7 | 0.687 | 0.684 | 0.574 | 0.416 | 0.419 | 0.491 | 0.428 | 0.415 |
| 2 | 8 | 0.708 | 0.630 | 0.807 | 0.487 | 0.476 | 0.816 | 0.646 | 0.467 |
| 3 | 9 | 0.568 | 0.558 | 0.818 | 0.280 | 0.339 | 0.478 | 0.459 | 0.376 |
| 4 | 10 | 0.570 | 0.471 | 0.535 | 0.249 | 0.424 | 0.460 | 0.293 | 0.577 |
| 5 | 11 | 0.672 | 0.649 | 0.612 | 0.237 | 0.439 | 0.527 | 0.522 | 0.525 |
| 6 | 12 | 0.701 | 0.481 | 0.617 | 0.307 | 0.528 | 0.609 | 0.685 | 0.648 |

| | | | | | | | |
|---|---|---|---|---|---|---|---|
| Standard | 0.084 | 0.121 | 0.100 | 0.063 | 0.134 | 0.145 | 0.102 |
| Deviation | | | | | | | |

Without Blank (No Cell) Subtraction (per ISO 10993-5)

| Average | 0.615 | 0.661 | 0.329 | 0.438 | 0.564 | 0.506 | 0.501 |
|---|---|---|---|---|---|---|---|
| Viability (%) | 100 | 107 | 54 | 71 | 92 | 82 | 82 |
| With Blank (No Cell) Subtraction | | | | Average Blank OD = | | | 0.052 |
| Average | 0.563 | 0.609 | 0.277 | 0.386 | 0.512 | 0.454 | 0.449 |
| Viability (%) | 100 | 103 | 49 | 68 | 91 | 81 | 80 |

Based on these results, the substrate material was determined to not have a cytotoxic effect.

### Unperforated Graphene on Substrate

Cytotoxicity of uncoated graphene disposed on the substrate was tested using the parameters in Table 3.

TABLE 3
Parameters used to prepare test and control articles for cytotoxicity
experiments with unperforated graphene disposed on the substrate

| Sample | Amount | Vehicle | Volume | Ratio | Time/Temperature |
|---|---|---|---|---|---|
| Test Article | 17.6 cm² | complete MEM | 2.9 mL | 6 cm²/mL | 24 ± 2 hours at 37 ± 1° C. |
| Positive Control | 30 cm² | complete MEM | 10.0 mL | 3 cm²/mL | 24 ± 2 hours at 37 ± 1° C. |
| Negative Control | 30 cm² | complete MEM | 10.0 mL | 3 cm²/mL | 24 ± 2 hours at 37 ± 1° C. |
| Untreated Control | N/A | complete MEM | 10.0 mL | N/A | 24 ± 2 hours at 37 ± 1° C. |

N/A: Not Applicable

The results of the cytotoxicity testing are shown in Table 4.

TABLE 4
Cell viability results in experiments with unperforated graphene disposed on the
substrate

| | | | | | | Test Article | | | |
|---|---|---|---|---|---|---|---|---|---|
| | | Control Articles | | | | 100% | | | |
| Replicates | | Untreated | | Negative | Positive | (neat) | 50% | 25% | 12.5% |
| 1 | 7 | 0.572 | 0.623 | 0.671 | 0.312 | 0.610 | 0.582 | 0.557 | 0.459 |
| 2 | 8 | 0.623 | 0.667 | 0.750 | 0.304 | 0.691 | 0.536 | 0.649 | 0.564 |
| 3 | 9 | 0.562 | 0.631 | 0.647 | 0.235 | 0.581 | 0.580 | 0.624 | 0.669 |
| 4 | 10 | 0.441 | 0.480 | 0.586 | 0.145 | 0.481 | 0.563 | 0.589 | 0.556 |
| 5 | 11 | 0.525 | 0.570 | 0.786 | 0.178 | 0.606 | 0.716 | 0.716 | 0.724 |
| 6 | 12 | 0.674 | 0.736 | 0.828 | 0.104 | 0.716 | 0.703 | 0.933 | 0.746 |

| | | | | | | | |
|---|---|---|---|---|---|---|---|
| Standard | 0.084 | 0.092 | 0.085 | 0.084 | 0.076 | 0.136 | 0.112 |
| Deviation | | | | | | | |

Without Blank (No Cell) Subtraction (per ISO 10993-5)

| Average | 0.592 | 0.711 | 0.213 | 0.614 | 0.613 | 0.678 | 0.620 |
|---|---|---|---|---|---|---|---|
| Viability (%) | 100 | 120 | 36 | 104 | 104 | 115 | 105 |
| With Blank (No Cell) Subtraction | | | | Average Blank OD = | | | 0.053 |
| Average | 0.539 | 0.658 | 0.160 | 0.561 | 0.560 | 0.625 | 0.567 |
| Viability (%) | 100 | 122 | 30 | 104 | 104 | 116 | 105 |

Based on these results, the unperforated graphene on substrate material was determined to not have a cytotoxic effect.

### Perforated Graphene on Substrate

Cytotoxicity of perforated graphene disposed on the substrate was tested using the parameters in Table 5.

TABLE 5
Parameters used to prepare test and control articles for cytotoxicity
experiments with perforated graphene disposed on the substrate

| Sample | Amount | Vehicle | Volume | Ratio | Time/Temperature |
|---|---|---|---|---|---|
| Test Article | 10.2 cm² | complete MEM | 1.7 mL | 6 cm²/mL | 24 ± 2 hours at 37 ± 1° C. |
| Positive Control | 30 cm² | complete MEM | 10.0 mL | 3 cm²/mL | 24 ± 2 hours at 37 ± 1° C. |
| Negative Control | 30 cm² | complete MEM | 10.0 mL | 3 cm²/mL | 24 ± 2 hours at 37 ± 1° C. |
| Untreated Control | N/A | complete MEM | 10.0 mL | N/A | 24 ± 2 hours at 37 ± 1° C. |

N/A: Not Applicable

The results of the cytotoxicity testing are shown in Table 6.

TABLE 6
Cell viability results in experiments with perforated graphene disposed on the substrate

| | | | | | | Test Article | | | |
|---|---|---|---|---|---|---|---|---|---|
| | | Control Articles | | | | 100% | | | |
| Replicates | | Untreated | | Negative | Positive | (neat) | 50% | 25% | 12.5% |
| 1 | 7 | 0.541 | 0.430 | 0.626 | 0.160 | 0.468 | 0.340 | 0.525 | 0.572 |
| 2 | 8 | 0.575 | 0.655 | 0.695 | 0.330 | 0.699 | 0.370 | 0.423 | 0.590 |
| 3 | 9 | 0.618 | 0.550 | 0.710 | 0.113 | 0.487 | 0.668 | 0.560 | 0.693 |
| 4 | 10 | 0.573 | 0.560 | 0.703 | 0.093 | 0.365 | 0.299 | 0.338 | 0.664 |
| 5 | 11 | 0.734 | 0.360 | 0.793 | 0.074 | 0.623 | 0.476 | 0.594 | 0.852 |
| 6 | 12 | 0.551 | 0.585 | 0.683 | 0.108 | 0.490 | 0.435 | 0.509 | 0.687 |

| | | | | | | | |
|---|---|---|---|---|---|---|---|
| Standard | 0.096 | 0.054 | 0.094 | 0.119 | 0.132 | 0.095 | 0.100 |
| Deviation | | | | | | | |

Without Blank (No Cell) Subtraction (per ISO 10993-5)

| Average | 0.561 | 0.702 | 0.146 | 0.522 | 0.431 | 0.492 | 0.676 |
|---|---|---|---|---|---|---|---|
| Viability (%) | 100 | 125 | 26 | 93 | 77 | 88 | 121 |
| With Blank (No Cell) Subtraction | | | | Average Blank OD = | | | 0.051 |
| Average | 0.510 | 0.651 | 0.095 | 0.471 | 0.380 | 0.441 | 0.625 |
| Viability (%) | 100 | 128 | 19 | 92 | 75 | 86 | 123 |

Based on these results, the perforated graphene on substrate material was determined to not have a cytotoxic effect.

### Example 2

### Implantation Testing

A muscle implantation test was used to asses local effects on living tissue when test materials or devices are implanted. Test materials measured approximately 1 mm in width and 10 mm in length. Test materials included (i) uncoated/bare substrate; (ii) unperforated graphene on substrate; and (iii) perforated graphene on substrate. The thickness of the materials was essentially the thickness of the polymer. Control strips also measured 1 mm in width and 10 mm in length.

Prior to implantation, the materials were sterilized in 70% ethanol. Then, 6 strips were implanted into each of the paravertebral muscles of a rabbit, approximately 2.5 cm from the midline and parallel to the spinal column and approximately 2.5 cm from each other. The test material was folded in half, so that the graphene-side (if applicable) was facing out (for uncoated/bare substrate a tape layer was applied, and the test material was implanted with the taped layer facing out) and implanted on one side of the spine. In a similar fashion, negative control strips were implanted in the contralateral muscle of each animal. A total of at least 10 test material strips and 10 control strips were required for evaluation.

The animals were maintained for 2 weeks under observation to ensure proper healing of implant sites and for clinical signs of toxicity. At the end of the 2-week period, the animals were weighed and sacrificed by an injectable barbiturate. Sufficient time was allowed to lapse for the tissue to be cut without bleeding.

The paravertebral muscles in which the test or control strips were implanted were excised in toto from each animal by slicing around the implant sites with a scalpel and lifting the tissue. Excised tissue was examined grossly and placed in containers with 10% neutral buffered formalin. Axillary lymph nodes were examined and found to be free of abnormalities, and so they were not collected.

Following fixation in formalin, each of the implant sites was excised from the larger mass of tissue and examined macroscopically for signs of inflammation, encapsulation, hemorrhaging, necrosis, and discoloration using the following scale: 0=normal; 1=mild; 2=moderate; and 3=severe. In all cases, the presence, form, and location of the implanted material appeared unchanged.

After macroscopic observation, the implant material was removed and a slide of tissue containing the implant site was processed. Histologic slides of hematoxylin and eosin stained sections were prepared and evaluated by light microscopic examination. The biological reaction (inflammatory responses and healing responses) were assessed by microscopic observation and the responses graded and recorded according to Tables 7 and 8.

TABLE 7
Inflammatory Responses

| | Score | | | | |
|---|---|---|---|---|---|
| Cell Type/Response | 0 | 1 | 2 | 3 | 4 |
| Polymorphonuclear Cells | 0 | Rare, 1-5/phf^{a} | 5-10/phf | Heavy Infiltrate | Packed |
| Lymphocytes | 0 | Rare, 1-5/phf | 5-10/phf | Heavy Infiltrate | Packed |
| Plasma Cells | 0 | Rare, 1-5/phf | 5-10/phf | Heavy Infiltrate | Packed |
| Macrophages | 0 | Rare, 1-5/phf | 5-10/phf | Heavy Infiltrate | Packed |
| Giant Cells | 0 | Rare, 1-2/phf | 3-5/phf | Heavy Infiltrate | Sheets |
| Necrosis | 0 | Minimal | Mild | Moderate | Severe |

^{a}phf = per high powered (400×) field.

TABLE 8
Healing Responses

| Cell | Score | | | | |
|---|---|---|---|---|---|
| Type/Response | 0 | 1 | 2 | 3 | 4 |
| Neovascularisation | 0 | Minimal capillary, | Groups of 4-7 | Broad band of | Extensive band |
| | | proliferation, | capillaries with | capillaries with | of capillaries with |
| | | focal, 1-3 buds | supporting | supporting | supporting |
| | | | fibroblastic | structures | fibroblastic |
| | | | structures | | structures |
| Fibrosis | 0 | Narrow band | Moderately thick | Thick band | Extensive band |
| | | | band | | |
| Fatty Infiltrate | 0 | Minimal amount | Several layers of | Elongated and | Extensive fat |
| | | of fat associated | fat and fibrosis | broad | completely |
| | | with fibrosis | | accumulation of | surrounding the |
| | | | | fat cells about the | implant |
| | | | | implant site | |

The relative size of the involved area was scored by assessing the width of the area from the implant/tissue interface to unaffected areas which have the characteristics of normal tissue and normal vascularity. Relative size of the involved area was scored using the following scale: 0=0 mm, no site; 1=up to 0.5 mm, very slight; 2=0.6-1.0 mm, mild; 3=1.1-2.0 mm, moderate; 4=>2.0 mm, severe.

For each implanted site, a total score was determined. The inflammatory responses were totaled for each site and weighted by a factor of 2. The healing responses were totaled separately. Inflammatory and healing responses were added together resulting in a total score for each site. The average score of the test sites was compared to the average score of the control sites for that animal. The average difference between the test and controls for all animals was calculated and a bioreactivity rating was assigned as follows: 0.0-2.9=no reaction (negative calculations were reported as 0); 3.0-8.9=slight reaction; 9.0-15.0=moderate reaction; and >15.0=severe reaction.

A pathologist reviewed the calculated level of reactivity. Based on the observation of all factors (e.g., relative size, pattern of response, inflammatory vs. resolution), the pathology observer was given leeway to revise the bioreactivity rating, if justified.

Implantation results are presented below for the following test materials: (i) uncoated/bare substrate; (ii) unperforated graphene on substrate; and (iii) perforated graphene on substrate.

### Uncoated/Bare Substrate

In implantation testing with uncoated/bare substrate, all three test animals decreased a biologically insignificant amount (less than 10%) in weight, as shown in Table 9.

TABLE 9
Animal weights and clinical observations - 2-week
implantation with uncoated/bare substrate

| | | Body Weight (kg) | | | |
|---|---|---|---|---|---|
| Animal | | Day 0 | Day 14 | Weight | Signs of |
| # | Sex | Nov. 10, 2015 | Nov. 24, 2015 | Change | Toxicity* |
| 50745 | Male | 4.45 | 4.45 | 0.00 | None |
| 50746 | Female | 4.27 | 3.85 | −0.42 | None |
| 50747 | Male | 4.19 | 4.09 | −0.10 | None |

*Summary of Clinical Observations from Day 0 through Day 14.

None of the animals exhibited signs of toxicity over the course of the study. Macroscopic evaluation indicated no significant signs of inflammation, encapsulation, hemorrhage, necrosis, or discoloration, as shown in Table 10.

TABLE 10
Macroscopic observations - 2-week implantation with uncoated/bare substrate

| | | | | | | | | | | | | | | |
|---|---|---|---|---|---|---|---|---|---|---|---|---|---|---|
| | | | | | | | Test | | | | | | | Control |
| Tissue Site | T1 | T2 | T3 | T4 | T5 | T6 | Average | C1 | C2 | C3 | C4 | C5 | C6 | Average |

Animal #: 50745

| | | | | | | | | | | | | | | |
|---|---|---|---|---|---|---|---|---|---|---|---|---|---|---|
| Inflammation | 0 | 0 | 0 | 0 | 0 | 0 | 0 | 0 | 0 | 0 | 0 | 0 | 0 | 0 |
| Encapsulation | 0 | 0 | 0 | 0 | 0 | 0 | 0 | 0 | 0 | 0 | 0 | 0 | 0 | 0 |
| Hemorrhage | 0 | 0 | 0 | 0 | 0 | 0 | 0 | 0 | 0 | 0 | 0 | 0 | 0 | 0 |
| Necrosis | 0 | 0 | 0 | 0 | 0 | 0 | 0 | 0 | 0 | 0 | 0 | 0 | 0 | 0 |
| Discoloration | 0 | 0 | 0 | 0 | 0 | 0 | 0 | 0 | 0 | 0 | 0 | 0 | 0 | 0 |
| Total | 0 | 0 | 0 | 0 | 0 | 1 | 0.18 | 0 | 0 | 0 | 0 | 0 | 0 | 0 |

Animal #: 50746

| | | | | | | | | | | | | | | |
|---|---|---|---|---|---|---|---|---|---|---|---|---|---|---|
| Inflammation | NSF | 0 | 0 | 0 | 0 | 0 | 0 | 0 | 0 | 0 | 0 | 0 | 0 | 0 |
| Encapsulation | NSF | 0 | 0 | 0 | 0 | 0 | 0 | 0 | 0 | 0 | 0 | 0 | 0 | 0 |
| Hemorrhage | NSF | 0 | 0 | 0 | 0 | 0 | 0 | 0 | 0 | 0 | 0 | 0 | 0 | 0 |
| Necrosis | NSF | 0 | 0 | 0 | 0 | 0 | 0 | 0 | 0 | 0 | 0 | 0 | 0 | 0 |
| Discoloration | NSF | 0 | 0 | 0 | 0 | 0 | 0 | 0 | 0 | 0 | 0 | 0 | 0 | 0 |
| Total | NSF | 0 | 0 | 0 | 0 | 0 | 0 | 0 | 0 | 0 | 0 | 0 | 0 | 0 |

Animal #: 50747

| | | | | | | | | | | | | | | |
|---|---|---|---|---|---|---|---|---|---|---|---|---|---|---|
| Inflammation | 0 | 0 | 0 | 0 | 0 | 0 | 0 | 0 | 0 | 0 | 0 | 0 | 0 | 0 |
| Encapsulation | 0 | 0 | 0 | 0 | 0 | 0 | 0 | 0 | 0 | 0 | 0 | 0 | 0 | 0 |
| Hemorrhage | 0 | 0 | 0 | 0 | 0 | 0 | 0 | 0 | 0 | 0 | 0 | 0 | 0 | 0 |
| Necrosis | 0 | 0 | 0 | 0 | 0 | 0 | 0 | 0 | 0 | 0 | 0 | 0 | 0 | 0 |
| Discoloration | 0 | 0 | 0 | 0 | 0 | 0 | 0 | 0 | 0 | 0 | 0 | 0 | 0 | 0 |
| Total | 0 | 0 | 0 | 0 | 0 | 0 | 0 | 0 | 0 | 0 | 0 | 0 | 0 | 0 |

# 50745 = T6: Found a control with the blue suture in muscle.
# 50747 = T3: Dark area not able to find yellow color area. May be “NSF”. Needs microscopic evaluation to decide.
Grading Scale
T = Test Site
C = Control Site
0 = No Reaction
1 = Mild Reaction
2 = Moderate Reaction
3 = Severe Reaction
NSF = No Site Found (Representative Section Submitted)
NA = Not Applicable

Microscopic evaluation of the implant sites indicated no significant signs of inflammation, fibrosis, neovascsularization, or fatty infiltrate as compared to control material sites, as shown in Table 11.

TABLE 11
Microscopic evaluations with uncoated/bare substrate

| Categories | Test Sites | | | | | | | Control Sites | | | | | |
|---|---|---|---|---|---|---|---|---|---|---|---|---|---|
| of Reaction | T1† | T2 | | T3 | T4† | T5† | T6 | C1 | C2 | C3 | C4 | C5 | C6 |
| Foreign Debria | 0 | NSF | ** | 0 | 0 | 0 | 0 | 0 | 0 | 0 | 0 | 0 | 0 |
| Rel. Size | 1 | NSF | | 1 | 1 | 1 | 1 | 1 | 1 | 1 | 1 | 1 | 1 |
| *Polymorphs | 0 | NSF | | 1 | 1 | 0 | 1 | 0 | 0 | 1 | 2 | 0 | 0 |
| *Lymphocytes | 0 | NSF | | 0 | 0 | 0 | 0 | 0 | 0 | 0 | 3 | 0 | 0 |
| *Plasma Cells | 0 | NSF | | 0 | 0 | 0 | 0 | 0 | 0 | 0 | 0 | 0 | 0 |
| *Macrophages | 1 | NSF | | 1 | 1 | 1 | 1 | 1 | 1 | 1 | 1 | 1 | 1 |
| *Giant Cells | 1 | NSF | | 1 | 1 | 0 | 1 | 0 | 0 | 0 | 0 | 0 | 0 |
| *Necrosis | 0 | NSF | | 0 | 0 | 0 | 0 | 0 | 0 | 0 | 0 | 0 | 0 |
| Subtotal (x2) | 4 | N/A | | 6 | 6 | 2 | 6 | 2 | 2 | 4 | 12 | 2 | 2 |
| *Neovascularization | 1 | NSF | | 1 | 1 | 1 | 1 | 1 | 1 | 1 | 1 | 1 | 1 |
| *Fibrosis | 1 | NSF | | 1 | 1 | 1 | 1 | 1 | 1 | 1 | 1 | 1 | 1 |
| *Fatty Infiltrate | 0 | NSF | | 0 | 0 | 0 | 0 | 0 | 0 | 0 | 0 | 0 | 0 |
| Subtotal (x1) | 2 | N/A | | 2 | 2 | 2 | 2 | 2 | 2 | 2 | 2 | 2 | 2 |
| Total | 6 | N/A | | 8 | 8 | 4 | 8 | 4 | 4 | 6 | 14 | 4 | 4 |

Animal Test Score (Average of Totals) = 6.8
Animal Control Score (Average of Totals) = 6.0
Animal Score (Average Test − Average Control) = 0.8
Comments:
**NSF = No Site Found
N/A = Not applicable
The test material in situ was not scored as foreign debris
†Some brown pigmented microphages and/or slight congestion at the interface were noted.
*Used in calculation of Bioreactivity Rating

Based on the above data, the bioreactivity rating was calculated to be 0.7.

### Unperforated Graphene on Substrate

In implantation testing with unperforated graphene disposed on the substrate, all three test animals decreased a biologically insignificant amount (less than 6%) in weight, as shown in Table 12.

TABLE 12
Animal weights and clinical observations - 2-Week
implantation with unperforated graphene on substrate

| | | Body Weight (kg) | | | |
|---|---|---|---|---|---|
| Animal | | Day 0 | Day 14 | Weight | Signs of |
| # | Sex | Nov. 10, 2015 | Nov. 24, 2015 | Change | Toxicity* |
| 50677 | Male | 3.93 | 3.73 | −0.20 | None |
| 50743 | Male | 3.77 | 3.66 | −0.11 | None |
| 50744 | Female | 4.05 | 3.83 | −0.22 | None |

*Summary of Clinical Observations from Day 0 through Day 14.

None of the animals exhibited signs of toxicity over the course of the study. Macroscopic evaluation indicated no significant signs of inflammation, encapsulation, hemorrhage, necrosis, or discoloration, as shown in Table 13.

TABLE 13
Macroscopic observations - 2-week implantation with unperforated graphene on
substrate

| | | | | | | | | | | | | | | |
|---|---|---|---|---|---|---|---|---|---|---|---|---|---|---|
| | | | | | | | Test | | | | | | | Control |
| Tissue Site | T1 | T2 | T3 | T4 | T5 | T6 | Average | C1 | C2 | C3 | C4 | C5 | C6 | Average |

Animal#: 50677

| | | | | | | | | | | | | | | |
|---|---|---|---|---|---|---|---|---|---|---|---|---|---|---|
| Inflammation | 0 | 0 | 0 | 0 | 0 | 0 | 0 | 0 | 0 | 0 | 0 | 0 | 0 | 0 |
| Encapsulation | 0 | 0 | 0 | 0 | 0 | 0 | 0 | 0 | 0 | 0 | 0 | 0 | 0 | 0 |
| Hemorrhage | 0 | 0 | 0 | 0 | 0 | 0 | 0 | 0 | 0 | 0 | 0 | 0 | 0 | 0 |
| Necrosis | 0 | 0 | 0 | 0 | 0 | 0 | 0 | 0 | 0 | 0 | 0 | 0 | 0 | 0 |
| Discoloration | 0 | 0 | 0 | 0 | 0 | 0 | 0 | 0 | 0 | 0 | 0 | 0 | 0 | 0 |
| Total | 0 | 0 | 0 | 0 | 0 | 0 | 0 | 0 | 0 | 0 | 0 | 0 | 0 | 0 |

Animal #: 50743

| | | | | | | | | | | | | | | |
|---|---|---|---|---|---|---|---|---|---|---|---|---|---|---|
| Inflammation | 0 | 0 | 0 | 0 | 0 | 0 | 0 | 0 | 0 | 0 | 0 | 0 | 0 | 0 |
| Encapsulation | 0 | 0 | 0 | 0 | 0 | 0 | 0 | 0 | 0 | 0 | 0 | 0 | 0 | 0 |
| Hemorrhage | 0 | 0 | 0 | 0 | 0 | 0 | 0 | 0 | 0 | 0 | 0 | 0 | 0 | 0 |
| Necrosis | 0 | 0 | 0 | 0 | 0 | 0 | 0 | 0 | 0 | 0 | 0 | 0 | 0 | 0 |
| Discoloration | 0 | 0 | 0 | 0 | 0 | 0 | 0 | 0 | 0 | 0 | 0 | 0 | 0 | 0 |
| Total | 0 | 0 | 0 | 0 | 0 | 0 | 0 | 0 | 0 | 0 | 0 | 0 | 0 | 0 |

Animal #: 50744

| | | | | | | | | | | | | | | |
|---|---|---|---|---|---|---|---|---|---|---|---|---|---|---|
| Inflammation | 0 | 0 | 0 | 0 | 0 | 0 | 0 | 0 | 0 | 0 | 0 | 0 | 0 | 0 |
| Encapsulation | 0 | 0 | 0 | 0 | 0 | 0 | 0 | 0 | 0 | 0 | 0 | 0 | 0 | 0 |
| Hemorrhage | 0 | 0 | 0 | 0 | 0 | 0 | 0 | 0 | 0 | 0 | 0 | 0 | 0 | 0 |
| Necrosis | 0 | 0 | 0 | 0 | 0 | 0 | 0 | 0 | 0 | 0 | 0 | 0 | 0 | 0 |
| Discoloration | 0 | 0 | 0 | 0 | 0 | 0 | 0 | 0 | 0 | 0 | 0 | 0 | 0 | 0 |
| Total | 0 | 0 | 0 | 0 | 0 | 0 | 0 | 0 | 0 | 0 | 0 | 0 | 0 | 0 |

Grading Scale
T = Test Site
C = Control Site
0 = No Reaction
1 = Mild Reaction
2 = Moderate Reaction
3 = Severe Reaction

Microscopic evaluation of the implant sites indicated no significant signs of inflammation, fibrosis, neovascsularization, or fatty infiltrate as compared to control material sites, as shown in Table 14. At one test site, a few MNGs were present only at the non-graphene side.

TABLE 14
Microscopic evaluations with unperforated graphene on substrate

| Categories | Test Sites | | | | | | Control Sites | | | | | |
|---|---|---|---|---|---|---|---|---|---|---|---|---|
| of Reaction | T1 | T2 | T3 | T4 | T5 | T6 | C1 | C2 | C3 | C4 | C5 | C6 |
| Foreign Debris | 0 | 0 | 0 | 0 | 0 | 0 | 0 | 0 | 0 | 0 | 0 | 0 |
| Rel. Size | 1 | 1 | 1 | 1 | 1 | 1 | 1 | 1 | 1 | 1 | 1 | 1 |
| *Polymorphs | 0 | 0 | 0 | 0 | 0 | 0 | 0 | 0 | 0 | 0 | 0 | 0 |
| *Lymphocytes | 0 | 0 | 0 | 0 | 0 | 0 | 0 | 0 | 0 | 0 | 0 | 0 |
| *Plasma Cells | 0 | 0 | 0 | 0 | 0 | 0 | 0 | 0 | 0 | 0 | 0 | 0 |
| *Macrophages | 1 | 0 | 1 | 1 | 1 | 1 | 0 | 0 | 0 | 0 | 0 | 0 |
| *Giant Cells | 0 | 1 | 0 | 0 | 0 | 1 | 0 | 0 | 0 | 0 | 0 | 0 |
| *Necrosis | 0 | 0 | 0 | 0 | 0 | 0 | 0 | 0 | 0 | 0 | 0 | 0 |
| Subtotal (x2) | 2 | 2 | 2 | 2 | 2 | 4 | 0 | 0 | 0 | 0 | 0 | 0 |
| *Neovascularization | 1 | 0 | 0 | 0 | 0 | 0 | 0 | 0 | 0 | 0 | 0 | 0 |
| *Fibrosis | 1 | 1 | 1 | 1 | 1 | 1 | 1 | 1 | 1 | 1 | 1 | 1 |
| *Fatty Infiltrate | 0 | 0 | 0 | 0 | 0 | 0 | 0 | 0 | 0 | 0 | 0 | 0 |
| Subtotal (x1) | 2 | 1 | 1 | 1 | 1 | 1 | 1 | 1 | 1 | 1 | 1 | 1 |
| Total | 4 | 3 | 3 | 3 | 3 | 5 | 1 | 1 | 1 | 1 | 1 | 1 |

Animal Test Score (Average of Totals) = 3.5
Animal Control Score (Average of Totals) = 1.0
Animal Score (Average Test − Average Control) = 2.5
Comments:
At one test site (Animal # 50677, T2), few MNGs were only present at non-G's side.
*Used in calculation of Bioreactivity Rating

Based on the above data, the bioreactivity rating was calculated to be 2.2.

### Perforated Graphene on Substrate

In implantation testing with perforated graphene disposed on the substrate, two animals lost between 3% and 10% of their body weights, and one animal maintained its weight, as shown in Table 15:

TABLE 15
Animal weights and clinical observations - 2-week
implantation with perforated graphene on substrate

| | | Body Weight (kg) | | | |
|---|---|---|---|---|---|
| | | Day 0 | Day 14 | Weight | Signs of |
| Animal # | Sex | Nov. 4, 2015 | Nov. 8, 2015 | Change | Toxicity* |
| 50769 | Male | 3.88 | 3.53 | −0.35 | None |
| 50763 | Male | 3.54 | 3.40 | −0.14 | None |
| 50772 | Female | 3.39 | 3.27 | −0.12 | None |

*Summary of Clinical Obervations from Day 0 through Day 14.

None of the animals exhibited signs of toxicity over the course of the study. Macroscopic evaluation indicated no significant signs of inflammation, encapsulation, hemorrhage, or necrosis, as shown in Table 16. Mild discoloration was noted in several control sites.

TABLE 16
Macroscopic observations - 2-week implantation with perforated graphene on
substrate

| | | | | | | | | | | | | | | |
|---|---|---|---|---|---|---|---|---|---|---|---|---|---|---|
| | | | | | | | Test | | | | | | | Control |
| Tissue Site | T1 | T2 | T3 | T4 | T5 | T6 | Average | C1 | C2 | C3 | C4 | C5 | C6 | Average |

Animal #: 50769

| | | | | | | | | | | | | | | |
|---|---|---|---|---|---|---|---|---|---|---|---|---|---|---|
| Inflammation | 0 | 0 | 0 | 0 | 0 | 0 | 0 | 0 | 0 | 0 | 0 | 0 | 0 | 0 |
| Encapsulation | 0 | 0 | 0 | 0 | 0 | 0 | 0 | 0 | 0 | 0 | 0 | 0 | 0 | 0 |
| Hemorrhage | 0 | 0 | 0 | 0 | 0 | 0 | 0 | 0 | 0 | 0 | 0 | 0 | 0 | 0 |
| Necrosis | 0 | 0 | 0 | 0 | 0 | 0 | 0 | 0 | 0 | 0 | 0 | 0 | 0 | 0 |
| Discoloration | 0 | 0 | 0 | 0 | 0 | 0 | 0 | 0 | 0 | 0 | 0 | 0 | 0 | 0 |
| Total | 0 | 0 | 0 | 0 | 0 | 0 | 0 | 0 | 0 | 0 | 0 | 0 | 0 | 0 |

Animal #: 50763

| | | | | | | | | | | | | | | |
|---|---|---|---|---|---|---|---|---|---|---|---|---|---|---|
| Inflammation | 0 | 0 | 0 | 0 | 0 | 0 | 0 | 0 | 0 | 0 | 0 | 0 | 0 | 0 |
| Encapsulation | 0 | 0 | 0 | 0 | 0 | 0 | 0 | 0 | 0 | 0 | 0 | 0 | 0 | 0 |
| Hemorrhage | 0 | 0 | 0 | 0 | 0 | 0 | 0 | 0 | 0 | 0 | 0 | 0 | 0 | 0 |
| Necrosis | 0 | 0 | 0 | 0 | 0 | 0 | 0 | 0 | 0 | 0 | 0 | 0 | 0 | 0 |
| Discoloration | 0 | 0 | 0 | 0 | 0 | 0 | 0 | 0 | 1 | 1 | 1 | 1 | 1 | 1 |
| Total | 0 | 0 | 0 | 0 | 0 | 0 | 0 | 0 | 1 | 1 | 1 | 1 | 1 | 1 |

Animal #: 50772

| | | | | | | | | | | | | | | |
|---|---|---|---|---|---|---|---|---|---|---|---|---|---|---|
| Inflammation | 0 | 0 | 0 | 0 | 0 | 0 | 0 | 0 | 0 | 0 | 0 | 0 | 0 | 0 |
| Encapsulation | 0 | 0 | 0 | 0 | 0 | 0 | 0 | 0 | 0 | 0 | 0 | 0 | 0 | 0 |
| Hemorrhage | 0 | 0 | 0 | 0 | 0 | 0 | 0 | 0 | 0 | 0 | 0 | 0 | 0 | 0 |
| Necrosis | 0 | 0 | 0 | 0 | 0 | 0 | 0 | 0 | 0 | 0 | 0 | 0 | 0 | 0 |
| Discoloration | 0 | 0 | 0 | 0 | 0 | 0 | 0 | 0 | 0 | 0 | 0 | 0 | 0 | 0 |
| Total | 0 | 0 | 0 | 0 | 0 | 0 | 0 | 0 | 0 | 0 | 0 | 0 | 0 | 0 |

Grading Scale
T = Test Site
C = Control Site
0 = NO Reaction
1 = Mild Reaction
2 = Moderate Reaction
3 = Severe Reaction
NSF = No Site Found (Representative Section Submitted)
N/A = Not Applicable

Microscopic evaluation of the implant sites indicated no significant signs of inflammation, fibrosis, neovascsularization, or fatty infiltrate as compared to control material sites, as shown in Table 17.

TABLE 17
Microscopic evaluations with perforated graphene on substrate

| Categories | Test Sites | | | | | | Control Sites | | | | | |
|---|---|---|---|---|---|---|---|---|---|---|---|---|
| of Reaction | T1† | T2† | T3† | T4 | T5† | T6 [symbol] | C1 | C2 | C3 | C4 | C5 | C6 |
| Foreign Debris | 0 | 0 | 0 | 0 | 0 | 0 | 0 | 0 | 0 | 0 | 0 | 0 |
| Rel. Size | 1 | 1 | 1 | 1 | 1 | 1 | 1 | 1 | 1 | 1 | 1 | 1 |
| *Polymorphs | 0 | 0 | 0 | 0 | 1 | 0 | 2 | 0 | 2 | 0 | 2 | 0 |
| *Lymphocytes | 0 | 0 | 0 | 0 | 0 | 0 | 0 | 0 | 1 | 0 | 0 | 0 |
| *Plasma Cells | 0 | 0 | 0 | 0 | 0 | 0 | 0 | 0 | 0 | 0 | 0 | 0 |
| *Macrophages | 1 | 1 | 1 | 1 | 1 | 1 | 1 | 1 | 1 | 1 | 1 | 1 |
| *Giant Cells | 1 | 0 | 0 | 0 | 0 | 0 | 0 | 0 | 0 | 0 | 0 | 0 |
| *Necrosis | 0 | 0 | 0 | 0 | 0 | 0 | 0 | 0 | 0 | 0 | 0 | 0 |
| Subtotal (x2) | 4 | 2 | 2 | 2 | 4 | 2 | 6 | 2 | 8 | 2 | 6 | 2 |
| *Neovascularization | 1 | 1 | 1 | 1 | 1 | 1 | 1 | 1 | 1 | 1 | 1 | 1 |
| *Fibrosis | 1 | 1 | 1 | 1 | 1 | 1 | 1 | 1 | 1 | 1 | 1 | 1 |
| *Fatty Infiltrate | 0 | 0 | 0 | 0 | 0 | 0 | 0 | 0 | 0 | 0 | 0 | 0 |
| Subtotal (x1) | 2 | 2 | 2 | 2 | 2 | 2 | 2 | 2 | 2 | 2 | 2 | 2 |
| Total | 6 | 4 | 4 | 4 | 6 | 4 | 8 | 4 | 10 | 4 | 8 | 4 |

Animal Test Score (Average of Totals) = 4.7
Animal Control Score (Average of Totals) = 6.3
Animal Score (Average Test − Average Control) = −1.6
Comments:
†Slight congestion at the interface was noted. [symbol]
Some brown pigmented macrophages at the interface were found.
*Used in calculation of Bioreactivity Rating [symbol]
indicates data missing or illegible when filed

Based on the above data, the bioreactivity rating was calculated to be 0.0.

### Example 3

### Permeability Study—Allura Red AC and Silver Nanoparticles

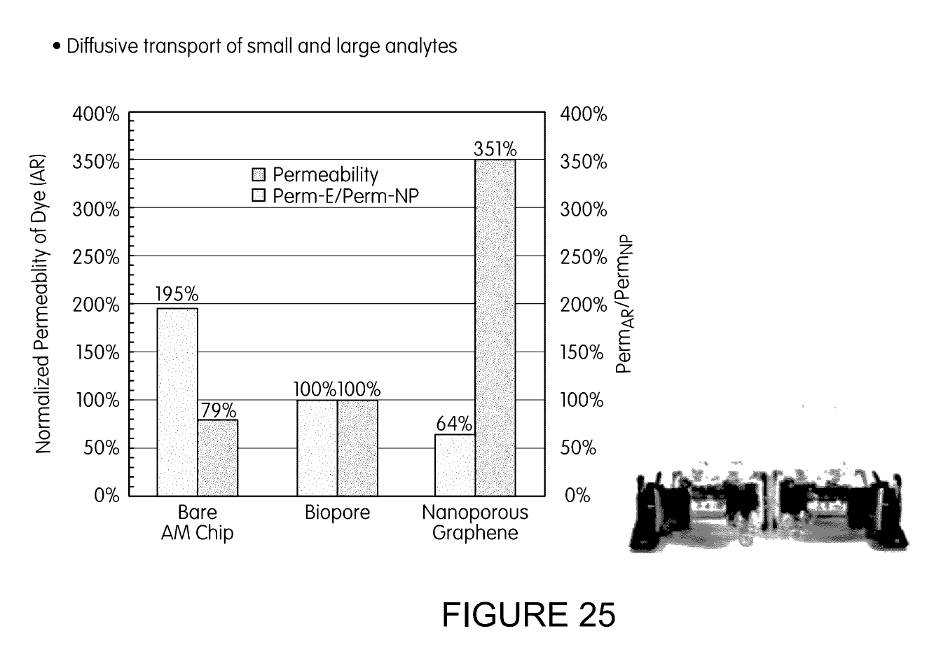

Permeability of small (Allura Red AC) and large (silver nanoparticles) across a SiN substrate layer and a perforated graphene layer were assessed via diffusion cell experiments. Permeability was compared to a control membrane with selectivity on the order of nanometers. Results are displayed in FIG. 25, where “Bare AM Chip” is the SiN substrate, “Biopore” is the control membrane, and “Nanoporous graphene” is perforated graphene. In the Figure, results were normalized to permeability of substances through the control membrane based on area of the tested substances. Also, permeability results for Allura Red AC and silver nanoparticles through the control membrane were also normalized to each other (based on the raw data, permeability of Allura Red AC through the control membrane high than permeability of silver nanoparticles through the control membrane). The data shows that less Allura Red AC permeated the perforated graphene layer than the SiN layer. Moreover, to an even larger extent, the perforated graphene layer restricted silver nanoparticles from traversing the layer, as compared to the SiN layer.

### Example 4

### Diffusive Transport of Fluorescein and IgG

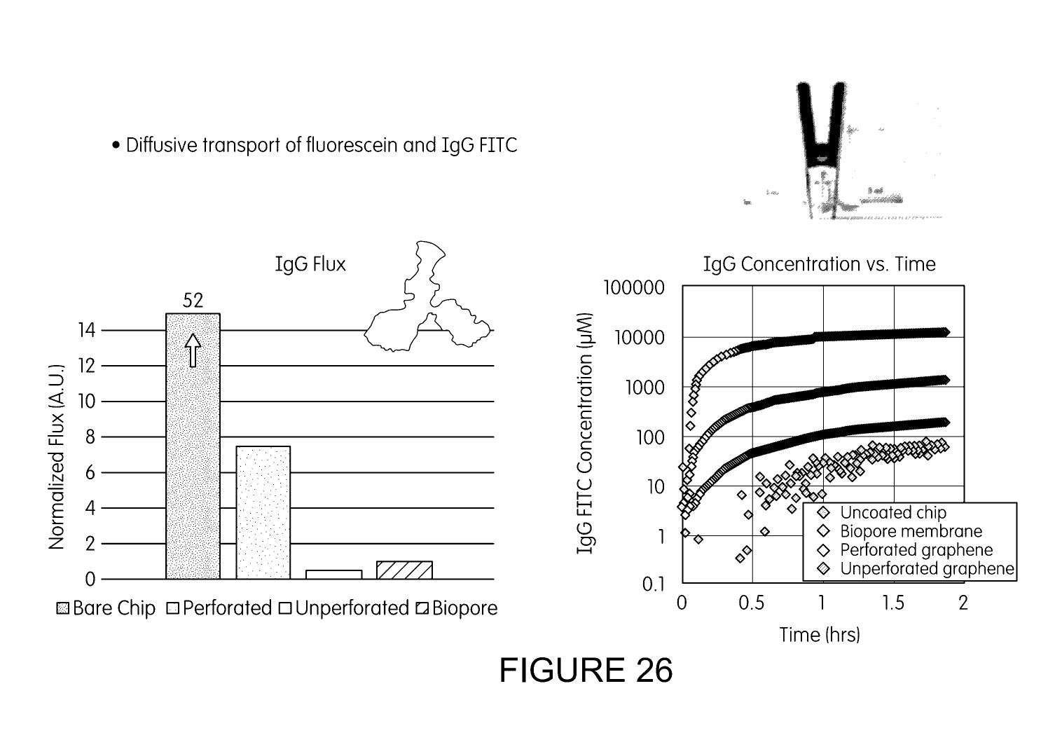

Diffusive transport of fluorescein conjugated to IgG was assessed via diffusion cell experiments with respect to the following materials: an SiN substrate layer (termed “Bare Chip” in FIG. 26), perforated graphene, unperforated graphene, and a control membrane (termed “Biopore” in the FIG. 26). The results showed that normalized flux of IgG through the SiN substrate layer was higher than through perforated graphene. The results also showed minimum flux of IgG through unperforated graphene. Without being bound by theory, it is believed that flux observed through unperforated graphene resulted from intrinsic or native defects in the graphene.

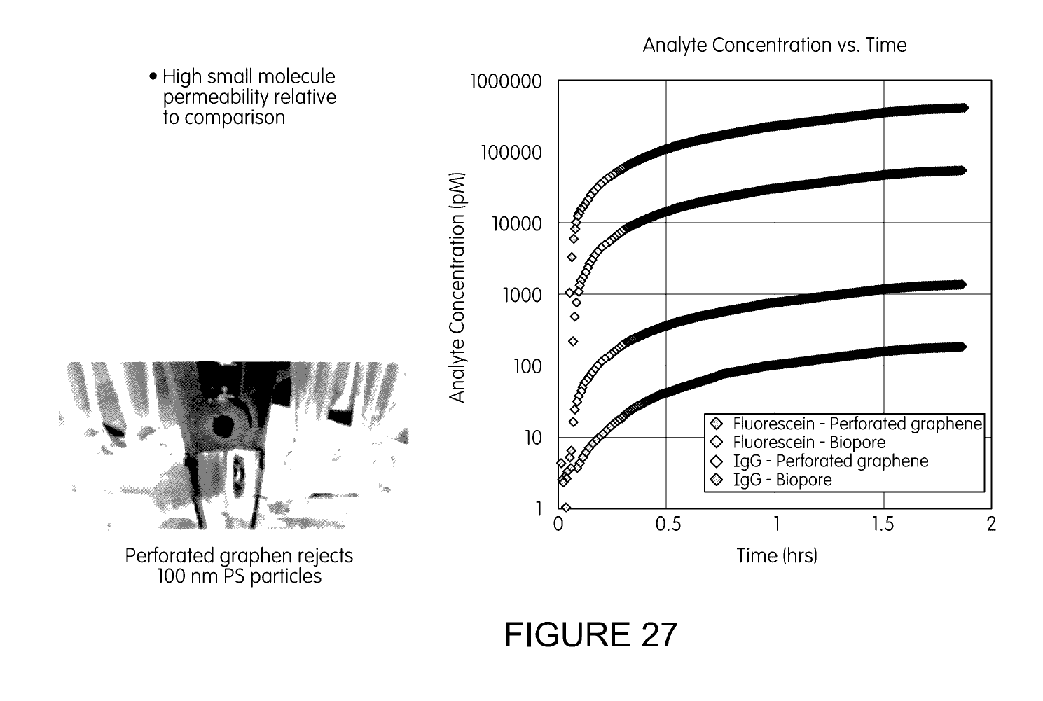

FIG. 27 compares the permeability results in FIG. 26 with permeability data obtained on fluorescein alone (i.e., not conjugated to IgG). The Figure demonstrates that fluorescein permeability was higher than IgG permeability.

### Example 5

### Permeability Study—FluoSpheres and Fluorescein

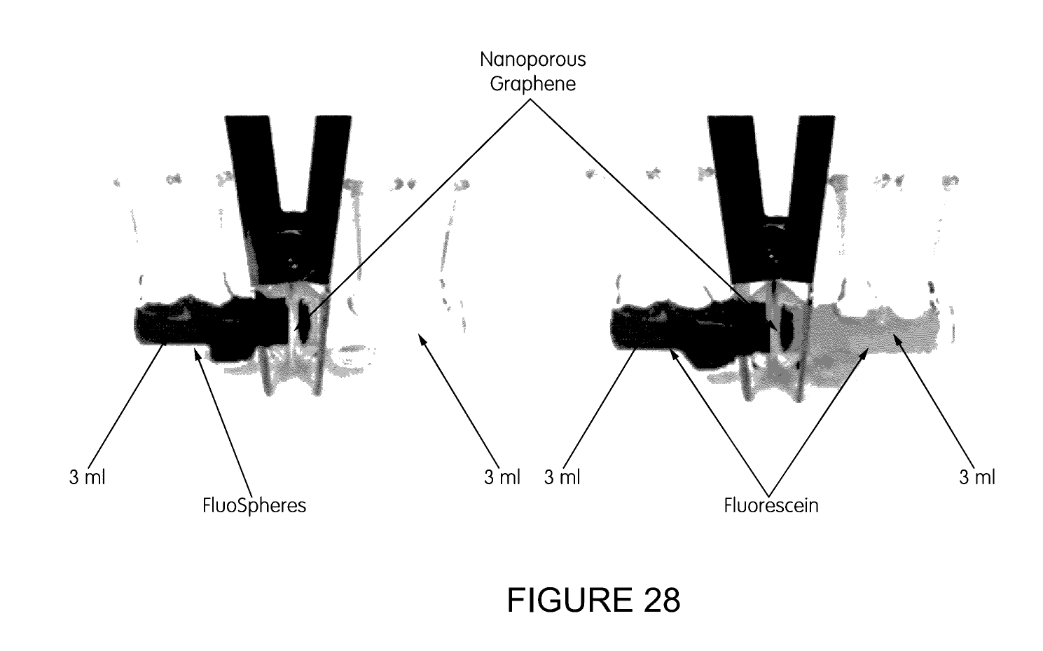

Permeability of 100 nm diameter Red (580/605) FluoSpheres and fluorescein was assessed via diffusion cell experiments with perforated graphene. As shown in FIG. 28, FluoSpheres added to the left side of diffusion cell did not traverse the perforated graphene. However, fluorescein added to the left side of the diffusion cell traversed the perforated graphene, and then was detected on the right side of the diffusion cell.

### Example 6

### Permeability Study—Fluorescein Across Various Substrates

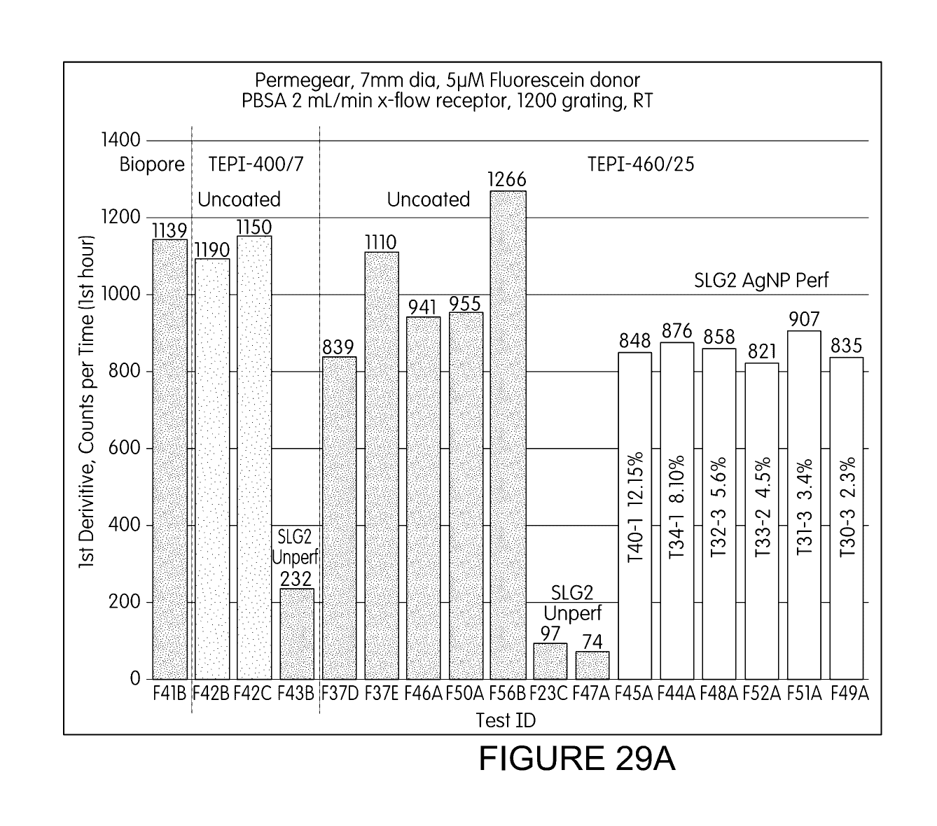

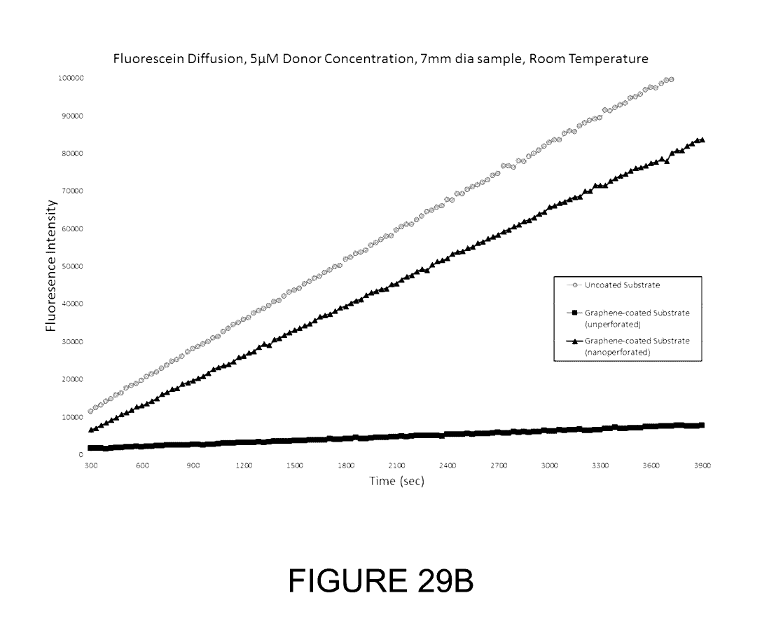

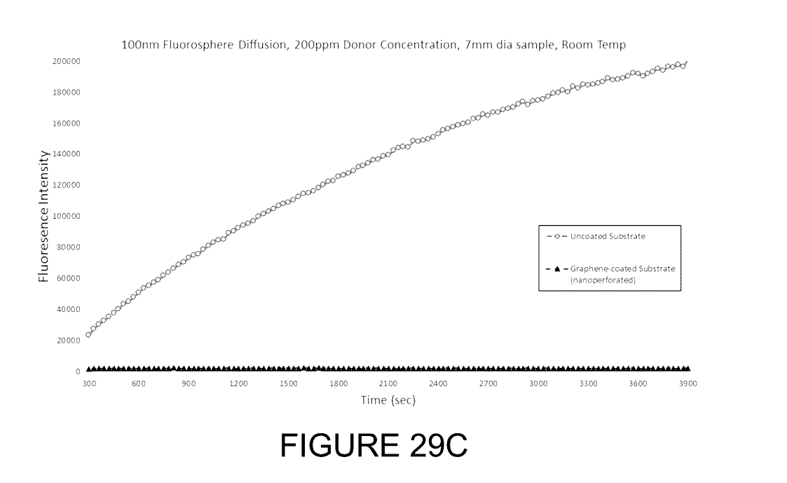

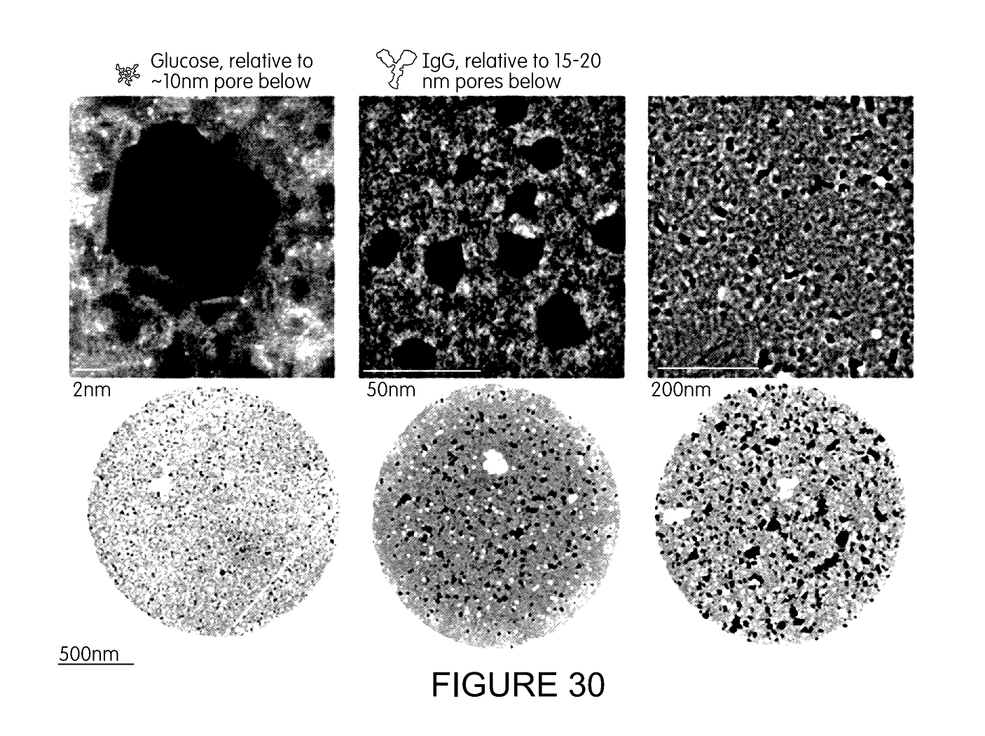

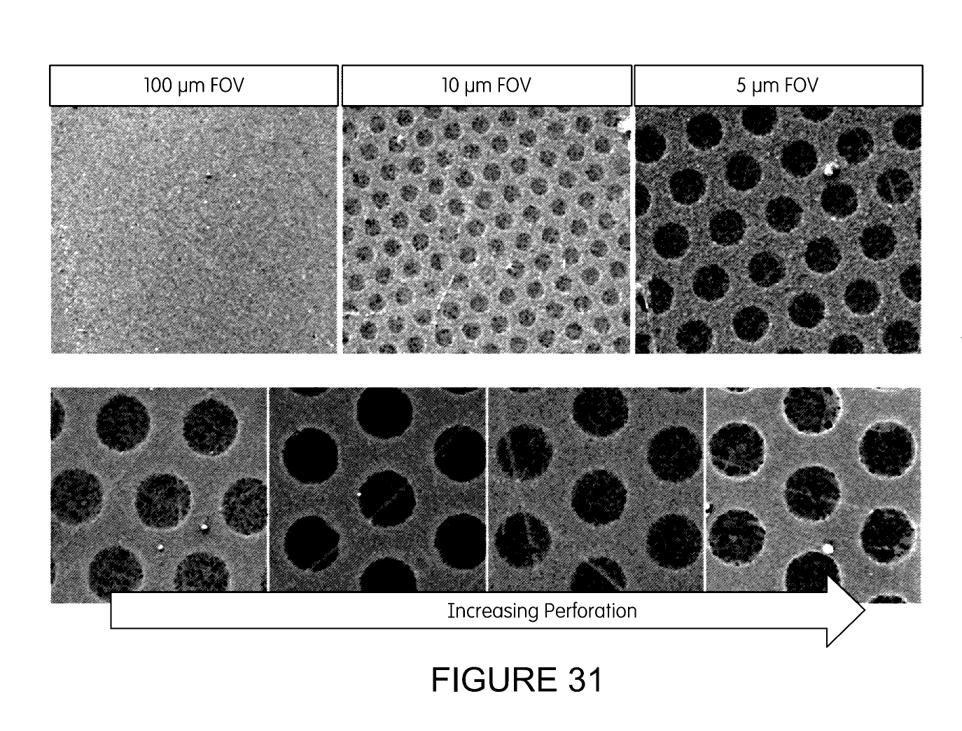

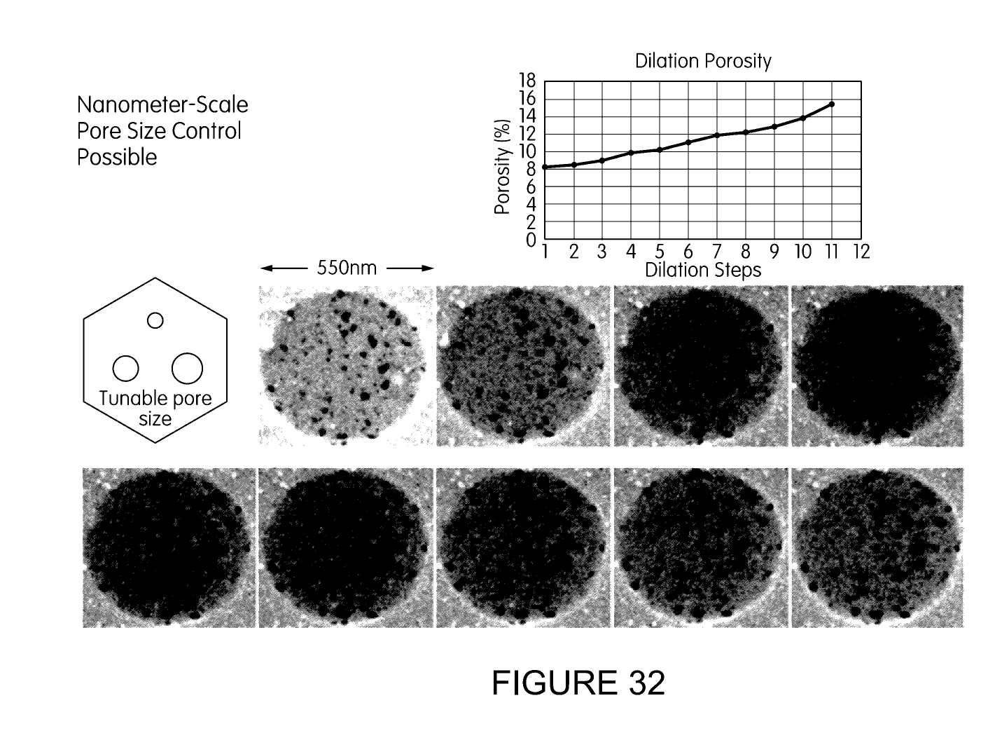

Permeability of fluorescein across various substrates was measured via Permegear cells. The experiments were conducted with 7 mm diameter test materials, and with 5 μM fluorescein in PB SA buffer at room temperature. As shown in FIG. 29A, the following test materials were used: (i) a control membrane (“Biopore”); (ii) uncoated substrate TEPI-400/7 (i.e., 400 nm pores, 7 μm thick substrate); (ii) TEPI-400/7 coated with two layers of unperforated graphene (“SLG2 Unperf”); (iii) uncoated substrate TEPI-460/25; (iv) TEPI-460/25 coated with two layers of unperforated graphene; and (v) TEPI-460/25 coated with two layers of perforated graphene, where the graphene was perforated with silver nanoparticles.

The data showed that unperforated graphene substantially reduced the amount of fluorescein that traversed the substrate layers. The data also showed the coating the substrate with perforated graphene did not substantially alter permeability of the substrate. That is, the permeability of fluorescein across uncoated TEPI-460/25 was similar to that of fluorescein across TEPI-460/25 coated with perforate graphene. This was the case even if only a small percentage of graphene suspended across the substrate was perforated. For instance, the data show similar results when 12-15%, 8-10%, 5-6%, 4-5%. 3-4% or 2-3% of the graphene suspended across the substrate was porous.

Additional data (not shown) further demonstrated that permeability of fluorescein across the substrate was enhanced by etching the substrate with NaOCl.

FIGS. 29B and 29C show diffusion for small fluorescent dye molecules (fluorescein, FIG. 29B) and large 100 nm FluoroSpheres (FIG. 29C) through uncoated substrate, unperforated graphene-coated substrate, and perforated graphene-coated substrate. The plots show the relative fluorescence intensity for the low concentration solution side of the membrane over time. The uncoated substrate was highly permeable to both fluorescent analytes, whereas the unperforated graphene-coated membrane had low permeablility. Size selectivity of the nanoperforated graphene membrane was demonstrated by its relatively high permeability to the smaller analyte (fluorescein, FIG. 29B) and low permeability to the larger analyte (FluoroSpheres, FIG. 29C). Both analytes were diluted in phosphate-buffered saline solution. Donor concentrations were 5 μM for fluorescein and 200 ppm for FluoroSpheres. The substrate was a track-etched polymeric membrane with pore diameters ranging between approximately 350-450 nm. Sample area available for diffusion was 49 mm². Testing was performed at room temperature.

1. An enclosure comprising a compartment and a wall separating the compartment from an environment external to the compartment, wherein the wall comprises:
a perforated graphene-based material layer and
a substrate layer affixed directly or indirectly to the perforated graphene-based material,
and wherein the enclosure is not cytotoxic when implanted into a subject.

2. The enclosure of claim 1, wherein the substrate layer comprises track-etched polyimide.

3. The enclosure of claim 1, wherein the substrate layer comprises a fibrous layer comprising a plurality of polymer filaments.

4. The enclosure of claim 1, wherein the compartment is in fluid communication with the external environment.

5. The enclosure of claim 1, wherein at least a portion of the wall is from about 5 nm to about 1 μm thick.

6. The enclosure of claim 1, further comprising at least one substance encapsulated within the compartment.

7. The enclosure of claim 6, wherein two or more substances are encapsulated within the compartment.

8. The enclosure of claim 6, wherein the substance comprises one or more cells, and wherein and the perforated graphene has pores with a size sufficient to retain the cell within the compartment and to exclude immune cells and immune complexes in the environment external to the compartment from entering the compartment.

9. The enclosure of claim 8, wherein the cells are mammalian cells.

10. The enclosure of claim 8, wherein the cells are yeast or bacterial cells.

11. The enclosure of claim 1, wherein the compartment does not contain sub-compartments.

12. The enclosure of claim 1, wherein the compartment comprises two or more sub-compartments.

13. The enclosure of claim 12, wherein one or more sub-compartments are separated from an environment external to the sub-compartment.

14. The enclosure of claim 13, wherein the one or more sub-compartments are separated from the environment external to the sub-compartment by a wall comprising a perforated graphene-based material.

15. The enclosure of claim 1, wherein the perforated graphene-based material has pores with a size sufficient to allow a pharmaceutical to pass between the compartment and the external environment.

16. The enclosure of claim 1, wherein the perforated graphene-based material has pores with a size of from about 1 nm to about 10 nm.

17. The enclosure of claim 1, wherein the graphene-based material is graphene.

18. The enclosure of claim 1, wherein the substrate layer is positioned on a compartment-facing side of the wall, on an exterior surface of the wall, or on both the compartment-facing side and exterior surface of the wall.

19. The enclosure of claim 18, wherein the substrate layer is disposed on the compartment-facing side of the graphene-based material, the external side of the graphene-based material, or both.

20. The enclosure of claim 1, wherein the substrate layer has a thickness of about 1 mm or less.

21. The enclosure of claim 1, wherein the substrate layer comprises a plurality of pores.

22. The enclosure of claim 0, wherein the substrate layer has a porosity gradient throughout its thickness.

23. The enclosure of claim 1, wherein the substrate layer is hydrophobic or hydrophilic.

24. The enclosure of claim 1, wherein the substrate layer comprises a material selected from the group consisting of polysulfones, polyurethane, polymethylmethacrylate (PMMA), polyglycolid acid (PGA), polylactic acid (PLA), polyethylene glycol (PEG), polylactic-co-glycolic acid (PLGA), polyamides, polyimides, polypropylene, polyethersulfones (PES), polyvinylidine fluoride (PVDF), cellulose acetate, polyethylene, polypropylene, polycarbonate, polytetrafluoroethylene (PTFE), polyvinylchloride (PVC), polyether ether ketone (PEEK), block co-polymers of any of these, and combinations and/or mixtures thereof.

25. The enclosure of claim 1, wherein the substrate layer comprises an additive selected from the group consisting of pharmaceuticals, cells, growth factors, clotting factors, blood thinners, immunosuppressants, antimicrobial agents, hormones, antibodies, antigens or an antibody-binding fragment thereof, minerals, nutrients, and combinations thereof.

26. The enclosure of claim 1, further comprising an intermediate layer positioned between the perforated graphene-based material layer and the substrate layer.

27. A method of releasing a substance comprising exposing an enclosure comprising a wall with a perforated graphene-based material layer and a substrate layer to an environment, to thereby release into the environment at least one substance from a compartment in the enclosure, wherein the enclosure is not cytotoxic to the environment.

28. The method of claim 27, wherein the environment is a biological environment.

29. The method of claim 27, wherein the substance is a pharmaceutical.

30. The method of claim 27, wherein the compartment contains cells which are not released from the enclosure.

31. The method of claim 27, wherein the cells produce the substance released from the enclosure.

32.-33. (canceled)

34. A composite structure comprising:
a perforated graphene-based material and a substrate layer affixed directly or indirectly to at least one surface of the perforated graphene-based material, wherein the composite structure is substantially planar, and wherein the composite structure is not cytotoxic when implanted into a subject.

35. The composite structure of claim 34, further comprising a second substrate layer affixed directly or indirectly to a surface of the perforated graphene-based material opposite the first substrate layer.

36. The composite structure of claim 35, wherein the first and/or second substrate layer comprises an additive selected from the group consisting of pharmaceuticals, cells, growth factors, clotting factors, blood thinners, immunosuppressants, antimicrobial agents, hormones, antibodies, antigens or an antibody-binding fragment thereof, minerals, nutrients and combinations thereof.

37. The composite structure of claim 34, wherein the composite structure is flexible.

38. An enclosure comprising a compartment and a wall separating the compartment from an environment external to the compartment, wherein the wall comprises:
a perforated graphene-based material layer and a means for enhancing integration of the enclosure into tissue and/or vascularization to the enclosure, wherein the enclosure is not cytotoxic when implanted into a subject.

39. A method of preparing an enclosure that is not cytotoxic when implanted into a subject comprising a compartment and a wall separating the compartment from an environment external to the compartment, wherein the wall comprises:
a perforated graphene-based material layer and
a substrate layer.

40. A method of improving biocompatibility of an enclosure comprising a perforated graphene-based material, wherein the method comprises affixing a substrate layer to the outside of the enclosure, wherein the enclosure is not cytotoxic when implanted into a subject.

41. (canceled)

42. A method of reducing cytotoxicity of a device comprising encapsulating the device with a composite structure comprising (a) a perforated graphene-based material layer and (b) a substrate layer affixed directly or indirectly to at least one surface of the perforated graphene-based material, wherein the device has a reduced cytotoxicity as compared to a comparable device not encapsulated by the composite structure.

43. A coated therapeutic device comprising:
(i) a therapeutic device and
(ii) a coating on the therapeutic device, wherein the coating comprises a composite structure comprising (a) a perforated graphene-based material layer and (b) a substrate layer affixed directly or indirectly to at least one surface of the perforated graphene-based material.

44. The coated therapeutic device of claim 43, wherein the coated therapeutic device has a lower toxicity than a comparable therapeutic device that is not coated with the composite structure.

45.-46. (canceled)
